SEQUENCE LISTING (55 sequences)

SEQ ID NO: 1 - DNA, 44 bp, Artificial Sequence
  Description of Artificial Sequence Synthetic primer
atggtgagcaagggcgaggagctgttcaccggggtggtgcccat

SEQ ID NO: 2 - DNA, 44 bp, Artificial Sequence
  Description of Artificial Sequence Synthetic oligonucleotide
atgtttagcaagggcgaggagctgttcaccggggtggtgcccat

SEQ ID NO: 3 - DNA, 44 bp, Artificial Sequence
  Description of Artificial Sequence Synthetic oligonucleotide
atgttaagcaagggcgaggagctgttcaccggggtggtgcccat

SEQ ID NO: 4 - DNA, 44 bp, Artificial Sequence
  Description of Artificial Sequence Synthetic oligonucleotide
atgattagcaagggcgaggagctgttcaccggggtggtgcccat

SEQ ID NO: 5 - DNA, 44 bp, Artificial Sequence
  Description of Artificial Sequence Synthetic oligonucleotide
atgtctagcaagggcgaggagctgttcaccggggtggtgcccat

SEQ ID NO: 6 - DNA, 44 bp, Artificial Sequence
  Description of Artificial Sequence Synthetic oligonucleotide
atgcctagcaagggcgaggagctgttcaccggggtggtgcccat

SEQ ID NO: 7 - DNA, 44 bp, Artificial Sequence
  Description of Artificial Sequence Synthetic oligonucleotide
atgactagcaagggcgaggagctgttcaccggggtggtgcccat

SEQ ID NO: 8 - DNA, 44 bp, Artificial Sequence
  Description of Artificial Sequence Synthetic oligonucleotide
atggctagcaagggcgaggagctgttcaccggggtggtgcccat

SEQ ID NO: 9 - DNA, 44 bp, Artificial Sequence
  Description of Artificial Sequence Synthetic oligonucleotide
atgtatagcaagggcgaggagctgttcaccggggtggtgcccat

SEQ ID NO: 10 - DNA, 44 bp, Artificial Sequence
  Description of Artificial Sequence Synthetic oligonucleotide
atgcatagcaagggcgaggagctgttcaccggggtggtgcccat

SEQ ID NO: 11 - DNA, 44 bp, Artificial Sequence
  Description of Artificial Sequence Synthetic oligonucleotide
atgcaaagcaagggcgaggagctgttcaccggggtggtgcccat

SEQ ID NO: 12 - DNA, 44 bp, Artificial Sequence
  Description of Artificial Sequence Synthetic oligonucleotide
atgaatagcaagggcgaggagctgttcaccggggtggtgcccat

SEQ ID NO: 13 - DNA, 44 bp, Artificial Sequence
  Description of Artificial Sequence Synthetic oligonucleotide
atgaaaagcaagggcgaggagctgttcaccggggtggtgcccat

SEQ ID NO: 14 - DNA, 44 bp, Artificial Sequence
  Description of Artificial Sequence Synthetic oligonucleotide
atggatagcaagggcgaggagctgttcaccggggtggtgcccat

SEQ ID NO: 15 - DNA, 44 bp, Artificial Sequence
  Description of Artificial Sequence Synthetic oligonucleotide
atggaaagcaagggcgaggagctgttcaccggggtggtgcccat

SEQ ID NO: 16 - DNA, 44 bp, Artificial Sequence
  Description of Artificial Sequence Synthetic oligonucleotide
atgtgtagcaagggcgaggagctgttcaccggggtggtgcccat

SEQ ID NO: 17 - DNA, 44 bp, Artificial Sequence
  Description of Artificial Sequence Synthetic oligonucleotide
atgtggagcaagggcgaggagctgttcaccggggtggtgcccat

SEQ ID NO: 18 - DNA, 44 bp, Artificial Sequence
  Description of Artificial Sequence Synthetic oligonucleotide
atgcgtagcaagggcgaggagctgttcaccggggtggtgcccat

SEQ ID NO: 19 - DNA, 44 bp, Artificial Sequence
  Description of Artificial Sequence Synthetic oligonucleotide
atgggtagcaagggcgaggagctgttcaccggggtggtgcccat

SEQ ID NO: 20 - DNA, 62 bp, Artificial Sequence
  Description of Artificial Sequence Synthetic oligonucleotide
agacaatcaaccatttggggtggacagccttgacctctagacttcggcattttttttttttt

SEQ ID NO: 21 - DNA, 112 bp, Artificial Sequence
  Description of Artificial Sequence Synthetic polynucleotide
cgggatccttatcgtcatcgtcgtacagatcccgacccatttgctgtccaccagtcatgctagccataccatgatgatgatgatgatgagaaccccgcattttttttttttt

SEQ ID NO: 22 - DNA, 19 bp, Artificial Sequence
  Description of Artificial Sequence Synthetic primer
atgcggggttctcatcatc

SEQ ID NO: 23 - DNA, 20 bp, Artificial Sequence
  Description of Artificial Sequence Synthetic primer
cgggatccttatcgtcatcg

SEQ ID NO: 24 - protein, 7 aa, Artificial Sequence
  Description of Artificial Sequence Synthetic peptide
AWIKREQ

SEQ ID NO: 25 - protein, 7 aa, Artificial Sequence
  Description of Artificial Sequence Synthetic peptide
  MOD_RES (1)..(1): Any amino acid
XWIKREQ

SEQ ID NO: 26 - protein, 7 aa, Artificial Sequence
  Description of Artificial Sequence Synthetic peptide
  MOD_RES (2)..(2): Any amino acid
AXIKREQ

SEQ ID NO: 27 - protein, 7 aa, Artificial Sequence
  Description of Artificial Sequence Synthetic peptide
  MOD_RES (3)..(3): Any amino acid
AWXKREQ

SEQ ID NO: 28 - protein, 7 aa, Artificial Sequence
  Description of Artificial Sequence Synthetic peptide
  MOD_RES (4)..(4): Any amino acid
AWIXREQ

SEQ ID NO: 29 - protein, 7 aa, Artificial Sequence
  Description of Artificial Sequence Synthetic peptide
  MOD_RES (5)..(5): Any amino acid
AWIKXEQ

SEQ ID NO: 30 - protein, 7 aa, Artificial Sequence
  Description of Artificial Sequence Synthetic peptide
  MOD_RES (6)..(6): Any amino acid
AWIKRXQ

SEQ ID NO: 31 - protein, 7 aa, Artificial Sequence
  Description of Artificial Sequence Synthetic peptide
  MOD_RES (7)..(7): Any amino acid
AWIKREX

SEQ ID NO: 32 - protein, 6 aa, Artificial Sequence
  Description of Artificial Sequence Synthetic 6xHis tag
HHHHHH

SEQ ID NO: 33 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 34 - DNA, 2271 bp, Homo sapiens
tgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 35 - DNA, 37 bp, Artificial Sequence
  Description of Artificial Sequence Synthetic oligonucleotide
cccctgccctcaacaagatggcttgccaactggccaa

SEQ ID NO: 36 - DNA, 37 bp, Artificial Sequence
  Description of Artificial Sequence Synthetic oligonucleotide
cccctgccctcaacaagatgtgctgccaactggccaa

SEQ ID NO: 37 - DNA, 37 bp, Artificial Sequence
  Description of Artificial Sequence Synthetic oligonucleotide
cccctgccctcaacaagatggattgccaactggccaa

SEQ ID NO: 38 - DNA, 37 bp, Artificial Sequence
  Description of Artificial Sequence Synthetic oligonucleotide
cccctgccctcaacaagatggagtgccaactggccaa

SEQ ID NO: 39 - DNA, 37 bp, Artificial Sequence
  Description of Artificial Sequence Synthetic oligonucleotide
cccctgccctcaacaagatgttctgccaactggccaa

SEQ ID NO: 40 - DNA, 37 bp, Artificial Sequence
  Description of Artificial Sequence Synthetic oligonucleotide
cccctgccctcaacaagatgggttgccaactggccaa

SEQ ID NO: 41 - DNA, 37 bp, Artificial Sequence
  Description of Artificial Sequence Synthetic oligonucleotide
cccctgccctcaacaagatgcactgccaactggccaa

SEQ ID NO: 42 - DNA, 37 bp, Artificial Sequence
  Description of Artificial Sequence Synthetic oligonucleotide
cccctgccctcaacaagatgatctgccaactggccaa

SEQ ID NO: 43 - DNA, 37 bp, Artificial Sequence
  Description of Artificial Sequence Synthetic oligonucleotide
cccctgccctcaacaagatgaagtgccaactggccaa

SEQ ID NO: 44 - DNA, 37 bp, Artificial Sequence
  Description of Artificial Sequence Synthetic oligonucleotide
cccctgccctcaacaagatgctgtgccaactggccaa

SEQ ID NO: 45 - DNA, 37 bp, Artificial Sequence
  Description of Artificial Sequence Synthetic oligonucleotide
cccctgccctcaacaagatgatgtgccaactggccaa

SEQ ID NO: 46 - DNA, 37 bp, Artificial Sequence
  Description of Artificial Sequence Synthetic oligonucleotide
cccctgccctcaacaagatgaactgccaactggccaa

SEQ ID NO: 47 - DNA, 37 bp, Artificial Sequence
  Description of Artificial Sequence Synthetic oligonucleotide
cccctgccctcaacaagatgccttgccaactggccaa

SEQ ID NO: 48 - DNA, 37 bp, Artificial Sequence
  Description of Artificial Sequence Synthetic oligonucleotide
cccctgccctcaacaagatgcagtgccaactggccaa

SEQ ID NO: 49 - DNA, 37 bp, Artificial Sequence
  Description of Artificial Sequence Synthetic oligonucleotide
cccctgccctcaacaagatgagatgccaactggccaa

SEQ ID NO: 50 - DNA, 37 bp, Artificial Sequence
  Description of Artificial Sequence Synthetic oligonucleotide
cccctgccctcaacaagatgagctgccaactggccaa

SEQ ID NO: 51 - DNA, 37 bp, Artificial Sequence
  Description of Artificial Sequence Synthetic oligonucleotide
cccctgccctcaacaagatgacctgccaactggccaa

SEQ ID NO: 52 - DNA, 37 bp, Artificial Sequence
  Description of Artificial Sequence Synthetic oligonucleotide
cccctgccctcaacaagatggtgtgccaactggccaa

SEQ ID NO: 53 - DNA, 37 bp, Artificial Sequence
  Description of Artificial Sequence Synthetic oligonucleotide
cccctgccctcaacaagatgtggtgccaactggccaa

SEQ ID NO: 54 - DNA, 37 bp, Artificial Sequence
  Description of Artificial Sequence Synthetic oligonucleotide
cccctgccctcaacaagatgtactgccaactggccaa

SEQ ID NO: 55 - protein, 10 aa, Artificial Sequence
  Description of Artificial Sequence Synthetic peptide
HHHCCHHCHH

(19) United States
(12) Patent Application Publication
(10) Pub. No.: US 2018/0282721 A1
(43) Pub. Date: Oct. 4, 2018

(54) DE NOVO SYNTHESIZED COMBINATORIAL NUCLEIC ACID LIBRARIES

(71) Applicant: TWIST BIOSCIENCE CORPORATION, San Francisco, CA (US)

(72) Inventors: Anthony COX, Mountain View, CA (US); Siyuan CHEN, San Mateo, CA (US); Charles LEDOGAR, San Francisco, CA (US); Dominique TOPPANI, San Francisco, CA (US)

(21) Appl. No.: 15/921,479

(22) Filed: Mar. 14, 2018

Related U.S. Application Data
(60) Provisional application No. 62/578,326, filed on Oct. 27, 2017.
(60) Provisional application No. 62/471,723, filed on Mar. 15, 2017.

(51) Int. Cl. C12N 15/10

(52) U.S. Cl. CPC C12N 15/1086

(57) ABSTRACT

Disclosed herein are methods for the generation of highly accurate nucleic acid libraries encoding for predetermined variants of a nucleic acid sequence. The degree of variation may be complete, resulting in a saturated variant library, or less than complete, resulting in a non-saturating library of variants. The variant nucleic acid libraries described herein may be designed for further processing by transcription or translation. The variant nucleic acid libraries described herein may be designed to generate variant RNA, DNA and/or protein populations. Further provided herein are method for identifying variant species with increased or decreased activities, with applications in regulating biological functions and the design of therapeutics for treatment or reduction of disease.

### CROSS-REFERENCE

This application claims the benefit of U.S. Provisional Application No. 62/578,326, filed on Oct. 27, 2017; and U.S. Provisional Application No. 62/471,723, filed on Mar. 15, 2017, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 13, 2018, is named 44854-729_201_SL.txt and is 18,419 bytes in size.

### BACKGROUND

The cornerstone of synthetic biology is the design, build, and test process—an iterative process that requires DNA, to be made accessible for rapid and affordable generation and optimization of these custom pathways and organisms. In the design phase, the A, C, T and G nucleotides that constitute DNA are formulated into the various gene sequences that would comprise the locus or the pathway of interest, with each sequence variant representing a specific hypothesis that will be tested. These variant gene sequences represent subsets of sequence space, a concept that originated in evolutionary biology and pertains to the totality of sequences that make up genes, genomes, transcriptome and proteome.

Many different variants are typically designed for each design-build-test cycle to enable adequate sampling of sequence space and maximize the probability of an optimized design. Though straightforward in concept, process bottlenecks around speed, throughput and quality of conventional synthesis methods dampen the pace at which this cycle advances, extending development time. The inability to sufficiently explore sequence space due to the high cost of acutely accurate DNA and the limited throughput of current synthesis technologies remains the rate-limiting step.

Beginning with the build phase, two processes are noteworthy: nucleic acid synthesis and gene synthesis. Historically, synthesis of different gene variants was accomplished through molecular cloning. While robust, this approach is not scalable. Early chemical gene synthesis efforts focused on producing a large number of polynucleotides with overlapping sequence homology. These were then pooled and subjected to multiple rounds of polymerase chain reaction (PCR), enabling concatenation of the overlapping polynucleotides into a full length double stranded gene. A number of factors hinder this method, including time-consuming and labor-intensive construction, requirement of high volumes of phosphoramidites, an expensive raw material, and production of nanomole amounts of the final product, significantly less than required for downstream steps, and a large number of separate polynucleotides required one 96 well plate to set up the synthesis of one gene.

Synthesizing of polynucleotides on microarrays provided a significant increase in the throughput of gene synthesis. A large number of polynucleotides could be synthesized on the microarray surface, then cleaved off and pooled together. Each polynucleotide destined for a specific gene contains a unique barcode sequence that enabled that specific subpopulation of polynucleotides to be depooled and assembled into the gene of interest. In this phase of the process, each subpool is transferred into one well in a 96 well plate, increasing throughput to 96 genes. While this is two orders of magnitude higher in throughput than the classical method, it still does not adequately support the design, build, test cycles that require thousands of sequences at one time due to a lack of cost efficiency and slow turnaround times.

### BRIEF SUMMARY

Provided herein are methods of synthesizing a variant nucleic acid library, comprising: (a) providing predetermined sequences encoding for at least 500 polynucleotide sequences, wherein the at least 500 polynucleotide sequences have a preselected codon distribution; (b) synthesizing a plurality of polynucleotides encoding for the at least 500 polynucleotide sequences; (c) assaying an activity for nucleic acids encoded by or proteins translated based on the plurality of polynucleotides; and (d) collecting results from the assay in step (c), wherein the collecting comprises collecting results of predetermined sequences associated with a negative or null result. Further provided herein are methods of synthesizing a variant nucleic acid library, wherein step (d) comprises collecting results for at least 80% of the predetermined sequences. Further provided herein are methods of synthesizing a variant nucleic acid library, wherein step (d) comprises collecting results for at least 90% of the predetermined sequences. Further provided herein are methods of synthesizing a variant nucleic acid library, wherein step (d) comprises collecting results for at least 100% of the predetermined sequences. Further provided herein are methods of synthesizing a variant nucleic acid library, wherein at least about 70% of a predicted diversity is represented. Further provided herein are methods of synthesizing a variant nucleic acid library, wherein at least about 90% of a predicted diversity is represented. Further provided herein are methods of synthesizing a variant nucleic acid library, wherein at least about 95% of a predicted diversity is represented. Further provided herein are methods of synthesizing a variant nucleic acid library, wherein at least 80% of the at least 500 polynucleotide sequences are a correct size. Further provided herein are methods of synthesizing a variant nucleic acid library, wherein at least about 80% of the at least 500 polynucleotide sequences are each present in the variant nucleic acid library in an amount within 2× of a mean frequency for each of the polynucleotide sequences in the library. Further provided herein are methods of synthesizing a variant nucleic acid library further comprising collecting results from the assay in step (c) for predetermined sequences associated with an enhanced or reduced activity. Further provided herein are methods of synthesizing a variant nucleic acid library, wherein the activity is cellular activity. Further provided herein are methods of synthesizing a variant nucleic acid library, wherein the cellular activity comprises reproduction, growth, adhesion, death, migration, energy production, oxygen utilization, metabolic activity, cell signaling, response to free radical damage, or any combination thereof. Further provided herein are methods of synthesizing a variant nucleic acid library, wherein the variant nucleic acid library encodes sequences for variant genes or fragments thereof. Further provided herein are methods of synthesizing a variant nucleic acid library, wherein the variant nucleic acid library encodes for at least a portion of an antibody, an enzyme, or a peptide. Further provided herein are methods of synthesizing a variant nucleic acid library, wherein the nucleic acid library encodes a guide RNA (gRNA). Further provided herein are methods of synthesizing a variant nucleic acid library, wherein the nucleic acid library encodes a siRNA, a shRNA, a RNAi, or a miRNA.

Provided herein are methods for generating a combinatorial library of nucleic acids, the method comprising: (a) designing predetermined sequences encoding for: (i) a first plurality of polynucleotides, wherein each polynucleotide of the first plurality of polynucleotides encodes for variant sequence compared to a single reference sequence and (ii) a second plurality of polynucleotides, wherein each polynucleotide of the second plurality of polynucleotides encodes for variant sequence compared to the single reference sequence; (b) synthesizing the first plurality of polynucleotides and the second plurality of polynucleotides; and (c) mixing the first plurality of polynucleotides and the second plurality of polynucleotides to form the combinatorial library of nucleic acids, wherein at least about 70% of a predicted diversity is represented. Further provided herein are methods for generating a combinatorial library of nucleic acids, wherein the combinatorial library is a non-saturating combinatorial library. Further provided herein are methods for generating a combinatorial library of nucleic acids, wherein the combinatorial library is a saturating combinatorial library. Further provided herein are methods for generating a combinatorial library of nucleic acids, wherein at least 10,000 polynucleotides are synthesized. Further provided herein are methods for generating a combinatorial library of nucleic acids, wherein a total number of polynucleotides for generation of the non-saturating combinatorial library is at least 25% less than the total number polynucleotides for generation of a saturating combinatorial library. Further provided herein are methods for generating a combinatorial library of nucleic acids, wherein at least 80% of variants are a correct size. Further provided herein are methods for generating a combinatorial library of nucleic acids, wherein at least about 90% of a predicted diversity is represented. Further provided herein are methods for generating a combinatorial library of nucleic acids, wherein at least about 95% of a predicted diversity is represented. Further provided herein are methods for generating a combinatorial library of nucleic acids, wherein the combinatorial library encodes for a first reference sequence or a second reference sequence. Further provided herein are methods for generating a combinatorial library of nucleic acids, wherein the combinatorial library when translated encodes for a protein library. Further provided herein are methods for generating a combinatorial library of nucleic acids, wherein the nucleic acids of the combinatorial library are inserted into vectors. Further provided herein are methods for generating a combinatorial library of nucleic acids further comprising performing PCR mutagenesis of a nucleic acid using the combinatorial library as primers for a PCR mutagenesis reaction. Further provided herein are methods for generating a combinatorial library of nucleic acids, wherein the combinatorial library encodes sequences for variant genes or fragments thereof. Further provided herein are methods for generating a combinatorial library of nucleic acids, wherein the combinatorial library encodes for at least a portion of an antibody, an enzyme, or a peptide. Further provided herein are methods for generating a combinatorial library of nucleic acids, wherein the combinatorial library encodes for at least a portion of a variable region or a constant region of the antibody. Further provided herein are methods for generating a combinatorial library of nucleic acids, wherein the combinatorial library encodes for at least one CDR region of the antibody. Further provided herein are methods for generating a combinatorial library of nucleic acids, wherein the combinatorial encodes for a CDR1, a CDR2, and a CDR3 on a heavy chain and a CDR1, a CDR2, and a CDR3 on a light chain of the antibody. Further provided herein are methods for generating a combinatorial library of nucleic acids, wherein the combinatorial library encodes for a guide RNA (gRNA).

Provided herein are methods of synthesizing a variant nucleic acid library, comprising: (a) providing predetermined sequences encoding for a plurality of polynucleotides, wherein the polynucleotides encode for a plurality of codons having a variant sequence compared to a single reference sequence; (b) selecting a distribution value for codons at a preselected position in the predetermined nucleic acid reference sequence; (c) providing machine instructions to randomly generate a set of nucleic acid sequences with a distribution value that aligns with the selected distribution value, wherein the set of nucleic acid sequences is less than the amount of nucleic acid sequences required to generate a saturating codon variant library; and (d) synthesizing the variant nucleic acid library with a preselected distribution, wherein at least about 70% of a predicted diversity is represented. Further provided herein are methods of synthesizing a variant nucleic acid library, wherein at least 80% of variants are a correct size. Further provided herein are methods of synthesizing a variant nucleic acid library, wherein at least about 90% of a predicted diversity is represented. Further provided herein are methods of synthesizing a variant nucleic acid library, wherein at least about 95% of a predicted diversity is represented. Further provided herein are methods of synthesizing a variant nucleic acid library, wherein the variant nucleic acid library when translated encodes for a protein library. Further provided herein are methods of synthesizing a variant nucleic acid library, wherein the nucleic acids of the variant nucleic acid library are inserted into vectors. Further provided herein are methods of synthesizing a variant nucleic acid library further comprising performing PCR mutagenesis of a nucleic acid using the variant nucleic acid library as primers for a PCR mutagenesis reaction. Further provided herein are methods of synthesizing a variant nucleic acid library, wherein a codon assignment is used for determining each codon of the plurality of codons having a variant sequence. Further provided herein are methods of synthesizing a variant nucleic acid library, wherein the codon assignment is based on frequency of the codon sequence in an organism. Further provided herein are methods of synthesizing a variant nucleic acid library, wherein the organism is at least one of an animal, a plant, a fungus, a protist, an archaeon, and a bacterium. Further provided herein are methods of synthesizing a variant nucleic acid library, wherein the codon assignment is based on a diversity of the codon sequence.

Provided herein are methods of synthesizing a variant nucleic acid library, comprising: (a) providing predetermined sequences encoding for a plurality of polynucleotides, wherein the polynucleotides encode for a codon having a variant sequence compared to a single reference sequence; (b) dividing the plurality of polynucleotides into 5′ fragments of polynucleotides and 3′ fragments of polynucleotides; (c) selecting a distribution value for a codon at a preselected position in the predetermined nucleic acid reference sequence; (d) providing machine instructions to randomly generate a set of nucleic acids with a distribution value that aligns with the selected distribution value, wherein the set of nucleic acids is less than the amount of nucleic acids required to generate a saturating nucleic acid library; (e) synthesizing the 5′ fragments of polynucleotides and the 3′ fragments of polynucleotides; and (f) mixing the 5′ fragments of polynucleotides and the 3′ fragments of polynucleotides to form the variant nucleic acid library, wherein at least about 70% of a predicted diversity is represented. Further provided herein are methods of synthesizing a variant nucleic acid library, wherein at least 10,000 polynucleotides are synthesized. Further provided herein are methods of synthesizing a variant nucleic acid library, wherein at least 80% of variants are a correct size. Further provided herein are methods of synthesizing a variant nucleic acid library, wherein at least about 90% of a predicted diversity is represented. Further provided herein are methods of synthesizing a variant nucleic acid library, wherein at least about 95% of a predicted diversity is represented. Further provided herein are methods of synthesizing a variant nucleic acid library, wherein the plurality of polynucleotides is divided into at least one of more than one 5′ fragments and more than one 3′ fragments. Further provided herein are methods of synthesizing a variant nucleic acid library, wherein the variant nucleic acid library when translated encodes for a protein library. Further provided herein are methods of synthesizing a variant nucleic acid library, wherein the nucleic acids of the variant nucleic acid library are inserted into vectors. Further provided herein are methods of synthesizing a variant nucleic acid library further comprising performing PCR mutagenesis of a nucleic acid using the variant nucleic acid library as primers for a PCR mutagenesis reaction. Further provided herein are methods of synthesizing a variant nucleic acid library further comprising identifying a variant sequence with an enhanced or reduced activity. Further provided herein are methods of synthesizing a variant nucleic acid library, wherein the activity is cellular activity. Further provided herein are methods of synthesizing a variant nucleic acid library, wherein the cellular activity comprises reproduction, growth, adhesion, death, migration, energy production, oxygen utilization, metabolic activity, cell signaling, response to free radical damage, or any combination thereof. Further provided herein are methods of synthesizing a variant nucleic acid library, wherein the variant nucleic acid library encodes sequences for variant genes or fragments thereof. Further provided herein are methods of synthesizing a variant nucleic acid library, wherein the variant nucleic acid library encodes for at least a portion of an antibody, an enzyme, or a peptide. Further provided herein are methods of synthesizing a variant nucleic acid library, wherein the variant nucleic acid library encodes for at least a portion of a variable region or a constant region of the antibody. Further provided herein are methods of synthesizing a variant nucleic acid library, wherein the variant nucleic acid library encodes for at least one CDR region of the antibody. Further provided herein are methods of synthesizing a variant nucleic acid library, wherein the variant nucleic acid library encodes for a CDR1, a CDR2, and a CDR3 on a heavy chain and a CDR1, a CDR2, and a CDR3 on a light chain of the antibody. Further provided herein are methods of synthesizing a variant nucleic acid library, wherein a number of different sequences synthesized in the variant nucleic acid library is in a range of 50 to 1,000,000. Further provided herein are methods of synthesizing a variant nucleic acid library, wherein a number of different sequences synthesized in the variant nucleic acid library is in a range of 500 to 25000. Further provided herein are methods of synthesizing a variant nucleic acid library, wherein a number of different sequences synthesized in the variant nucleic acid library is in a range of 1000 to 15000. Further provided herein are methods of synthesizing a variant nucleic acid library further comprising performing PCR mutagenesis of a nucleic acid using the variant nucleic acid library as primers for a PCR mutagenesis reaction. Further provided herein are methods of synthesizing a variant nucleic acid library, wherein a codon assignment is used for determining the codon having a variant sequence. Further provided herein are methods of synthesizing a variant nucleic acid library, wherein the codon assignment is based on frequency of the codon sequence in an organism. Further provided herein are methods of synthesizing a variant nucleic acid library, wherein the organism is at least one of an animal, a plant, a fungus, a protist, an archaeon, and a bacterium. Further provided herein are methods of synthesizing a variant nucleic acid library, wherein the codon assignment is based on a diversity of the codon sequence. Further provided herein are methods of synthesizing a variant nucleic acid library, wherein the nucleic acid library encodes a guide RNA (gRNA).

Provided herein are methods for generating a combinatorial library of nucleic acids, the method comprising: (a) providing predetermined sequences encoding for: (i) a first plurality of polynucleotides, wherein each polynucleotide of the first plurality of polynucleotides encodes for a variant sequence compared to a single reference sequence and (ii) a second plurality of polynucleotides, wherein each polynucleotide of the second plurality of polynucleotides encodes for a variant sequence compared to the single reference sequence; (b) providing a structure having a surface; (c) synthesizing the first plurality of polynucleotides, wherein each polynucleotide of the first plurality of polynucleotides extends from the surface; (d) synthesizing the second plurality of polynucleotides, wherein each polynucleotide of the second plurality of polynucleotides extends from the surface; (e) releasing the first plurality of polynucleotides and the second plurality of polynucleotides from the surface; and (f) mixing the first plurality of polynucleotides and the second plurality of polynucleotides to form the combinatorial library of nucleic acids, wherein at least about 70% of a predicted diversity is represented. Further provided herein are methods for generating a combinatorial library of nucleic acids, wherein at least about 90% of a predicted diversity is represented. Further provided herein are methods for generating a combinatorial library of nucleic acids, wherein at least about 95% of a predicted diversity is represented.

Provided herein are methods of synthesizing a variant nucleic acid library, comprising: (a) designing predetermined sequences encoding for a plurality of polynucleotides, wherein the polynucleotides encode for a plurality of codons having a variant sequence compared to a single reference sequence; (b) synthesizing the plurality of polynucleotides to generate the variant nucleic acid library, wherein at least about 70% of a predicted diversity is represented; (c) expressing the variant nucleic acid library; and (d) evaluating an activity associated with variant nucleic acid library. Further provided herein are methods of synthesizing a variant nucleic acid library, wherein at least about 90% of a predicted diversity is represented. Further provided herein are methods of synthesizing a variant nucleic acid library, wherein at least about 95% of a predicted diversity is represented.

Provided herein are methods for generating a combinatorial library of nucleic acids, the method comprising: (a) providing predetermined sequences encoding for: (i) a first plurality of non-identical polynucleotides, wherein each non-identical polynucleotide of the first plurality of non-identical polynucleotides encodes for a variant sequence compared to a single reference sequence and (ii) a second plurality of non-identical polynucleotides, wherein each non-identical polynucleotide of the second plurality of non-identical polynucleotides encodes for a variant sequence compared to the single reference sequence; (b) providing a structure having a surface; (c) synthesizing the first plurality of non-identical polynucleotides, wherein each non-identical polynucleotide of the first plurality of non-identical polynucleotides extends from the surface; (d) synthesizing the second plurality of non-identical polynucleotides, wherein each non-identical polynucleotide of the second plurality of non-identical polynucleotides extends from the surface; (e) releasing the first plurality of non-identical polynucleotides and the second plurality of non-identical polynucleotides from the surface; and (f) mixing the first plurality of polynucleotides and the second plurality of polynucleotides to form the combinatorial library of nucleic acids, wherein at least about 70% of a predicted diversity is represented. Provided herein are methods for generating a combinatorial library of nucleic acids, wherein the combinatorial library is a non-saturating combinatorial library. Provided herein are methods for generating a combinatorial library of nucleic acids, wherein the combinatorial library is a saturating combinatorial library. Provided herein are methods for generating a combinatorial library of nucleic acids, wherein at least 10,000 polynucleotides are synthesized. Provided herein are methods for generating a combinatorial library of nucleic acids, wherein a total number of polynucleotides for generation of the non-saturating combinatorial library is at least 25% less than the total number polynucleotides for generation of a saturating combinatorial library. Provided herein are methods for generating a combinatorial library of nucleic acids, wherein at least 80% of variants are a correct size. Provided herein are methods for generating a combinatorial library of nucleic acids, wherein the variant combinatorial library encodes for a first reference sequence or a second reference sequence. Provided herein are methods for generating a combinatorial library of nucleic acids, wherein the combinatorial library when translated encodes for a protein library. Provided herein are methods for generating a combinatorial library of nucleic acids, wherein the nucleic acids of the combinatorial library are inserted into vectors. Provided herein are methods for generating a combinatorial library of nucleic acids further comprising performing PCR mutagenesis of a nucleic acid using the combinatorial library as primers for a PCR mutagenesis reaction. Provided herein are methods for generating a combinatorial library of nucleic acids, wherein the combinatorial library encodes sequences for variant genes or fragments thereof. Provided herein are methods for generating a combinatorial library of nucleic acids, wherein the combinatorial library encodes for at least a portion of an antibody, enzyme, or peptide. Provided herein are methods for generating a combinatorial library of nucleic acids, wherein the combinatorial library encodes for at least a portion of a variable region or constant region of the antibody. Provided herein are methods for generating a combinatorial library of nucleic acids, wherein the combinatorial library encodes for at least one CDR region of the antibody. Provided herein are methods for generating a combinatorial library of nucleic acids, wherein the combinatorial encodes for a CDR1, CDR2, and CDR3 on a heavy chain and CDR1, CDR2, and CDR3 on a light chain of the antibody. Provided herein are methods for generating a combinatorial library of nucleic acids, wherein the combinatorial library encodes for guide RNA (gRNA). Provided herein are methods for generating a combinatorial library of nucleic acids, wherein the combinatorial library has an aggregate error rate of less than 1 in 1000 bases compared to predetermined sequences. Provided herein are methods for generating a combinatorial library of nucleic acids, wherein the structure is a solid support, gel, or beads, and wherein the solid support is a plate or a column.

Provided herein are methods of synthesizing a variant nucleic acid library, comprising: (a) providing predetermined sequences encoding for a plurality of non-identical polynucleotides, wherein the non-identical polynucleotides encode for a plurality of codons having a variant sequence compared to a single reference sequence; (b) selecting a distribution value for codons at a preselected position in the predetermined nucleic acid reference sequence; (c) providing machine instructions to randomly generate a set of nucleic acids, wherein the set of nucleic acids is less than the amount of nucleic acids required to generate a saturating codon variant library; and (d) synthesizing a nucleic acid library with a preselected distribution, wherein at least about 70% of a predicted diversity is represented. Provided herein are methods of synthesizing a variant nucleic acid library, wherein at least 80% of variants are a correct size. Provided herein are methods of synthesizing a variant nucleic acid library, wherein the combinatorial library when translated encodes for a protein library. Provided herein are methods of synthesizing a variant nucleic acid library, wherein the nucleic acids of the combinatorial library are inserted into vectors. Provided herein are methods of synthesizing a variant nucleic acid library further comprising performing PCR mutagenesis of a nucleic acid using the combinatorial library as primers for a PCR mutagenesis reaction. Provided herein are methods of synthesizing a variant nucleic acid library, wherein a codon assignment is used for determining each codon of the plurality of codons having a variant sequence. Provided herein are methods of synthesizing a variant nucleic acid library, wherein the codon assignment is based on frequency of the codon sequence in an organism. Provided herein are methods of synthesizing a variant nucleic acid library, wherein the organism is at least one of an animal, plant, fungus, protist, archaeon, and bacterium. Provided herein are methods of synthesizing a variant nucleic acid library, wherein the codon assignment is based on a diversity of the codon sequence.

Provided herein are methods of synthesizing a variant nucleic acid library, comprising: (a) providing predetermined sequences encoding for a plurality of non-identical polynucleotides, wherein the non-identical polynucleotides encode for a codon having a variant sequence compared to a single reference sequence; (b) dividing the plurality of non-identical polynucleotides into 5′ fragments of non-identical polynucleotides and 3′ fragments of non-identical polynucleotides; (c) selecting a distribution value for a codon at a preselected position in the predetermined nucleic acid reference sequence; (d) providing machine instructions to randomly generate a set of nucleic acids, wherein the set of nucleic acids is less than the amount of nucleic acids required to generate a saturating nucleic acid library; (e) synthesizing the 5′ fragments of non-identical polynucleotides and the 3′ fragments of non-identical polynucleotides; and (f) mixing the 5′ fragments of non-identical polynucleotides and the 3′ fragments of non-identical polynucleotides to form the variant nucleic acid library, wherein at least about 70% of a predicted diversity is represented. Provided herein are methods of synthesizing a variant nucleic acid library, wherein at least 10,000 non-identical polynucleotides are synthesized. Provided herein are methods of synthesizing a variant nucleic acid library, wherein at least 80% of variants are a correct size. Provided herein are methods of synthesizing a variant nucleic acid library, wherein the plurality of non-identical polynucleotides are divided into at least one of more than one 5′ fragments and more than one 3′ fragments. Provided herein are methods of synthesizing a variant nucleic acid library, wherein the combinatorial library when translated encodes for a protein library. Provided herein are methods of synthesizing a variant nucleic acid library, wherein the nucleic acids of the combinatorial library are inserted into vectors. Provided herein are methods of synthesizing a variant nucleic acid library further comprising performing PCR mutagenesis of a nucleic acid using the combinatorial library as primers for a PCR mutagenesis reaction. Provided herein are methods of synthesizing a variant nucleic acid library further comprising identifying a variant sequence with an enhanced or reduced activity. Provided herein are methods of synthesizing a variant nucleic acid library, wherein the activity is cellular activity. Provided herein are methods of synthesizing a variant nucleic acid library, wherein the cellular activity comprises reproduction, growth, adhesion, death, migration, energy production, oxygen utilization, metabolic activity, cell signaling, response to free radical damage, or any combination thereof. Provided herein are methods of synthesizing a variant nucleic acid library, wherein the nucleic acid library encodes sequences for variant genes or fragments thereof. Provided herein are methods of synthesizing a variant nucleic acid library, wherein the nucleic acid library encodes for at least a portion of an antibody, enzyme, or peptide. Provided herein are methods of synthesizing a variant nucleic acid library, wherein the nucleic acid library encodes a guide RNA (gRNA). Provided herein are methods of synthesizing a variant nucleic acid library, wherein the nucleic acid library encodes for at least a portion of a variable region or constant region of the antibody. Provided herein are methods of synthesizing a variant nucleic acid library, wherein the nucleic acid library encodes for at least one CDR region of the antibody. Provided herein are methods of synthesizing a variant nucleic acid library, wherein the nucleic acid library encodes for CDR1, CDR2, and CDR3 on a heavy chain and CDR1, CDR2, and CDR3 on a light chain of the antibody. Provided herein are methods of synthesizing a variant nucleic acid library, wherein the nucleic acid library has an aggregate error rate of less than 1 in 1000 bases compared to predetermined sequences for a plurality of non-identical polynucleotides. Provided herein are methods of synthesizing a variant nucleic acid library, wherein a number of different sequences synthesized in the nucleic acid library is in a range of about 50 to about 1,000,000. Provided herein are methods of synthesizing a variant nucleic acid library, wherein a number of different sequences synthesized in the nucleic acid library is in a range of about 500 to about 25000. Provided herein are methods of synthesizing a variant nucleic acid library, wherein a number of different sequences synthesized in the nucleic acid library is in a range of about 1000 to about 15000. Provided herein are methods of synthesizing a variant nucleic acid library further comprising performing PCR mutagenesis of a nucleic acid using the combinatorial library as primers for a PCR mutagenesis reaction. Provided herein are methods of synthesizing a variant nucleic acid library, wherein a codon assignment is used for determining the codon having a variant sequence. Provided herein are methods of synthesizing a variant nucleic acid library, wherein the codon assignment is based on frequency of the codon sequence in an organism. Provided herein are methods of synthesizing a variant nucleic acid library, wherein the organism is at least one of an animal, plant, fungus, protist, archaeon, and bacterium. Provided herein are methods of synthesizing a variant nucleic acid library, wherein the codon assignment is based on a diversity of the codon sequence.

Provided herein are methods of synthesizing a variant nucleic acid library, comprising: (a) designing predetermined sequences encoding for a plurality of non-identical polynucleotides, wherein the non-identical polynucleotides encode for a plurality of codons having a variant sequence compared to a single reference sequence; (b) synthesizing the plurality of non-identical polynucleotides to generate the variant nucleic acid library, wherein at least about 70% of a predicted diversity is represented; (c) expressing the variant nucleic acid library; and (d) evaluating an activity associated with variant nucleic acid library.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 depicts a schematic for generation of a non-saturating combinatorial library.

FIG. 2 depicts a schematic for generation of a saturating combinatorial library.

FIGS. 3A-3D depict a process workflow for the synthesis of variant biological molecules incorporating a PCR mutagenesis step.

FIGS. 4A-4D depict a process workflow for the generation of a nucleic acid comprising a nucleic acid sequence which differs from a reference nucleic acid sequence at a single predetermined codon site.

FIGS. 5A-5F depict an alternative workflow for the generation of a set of nucleic acid variants from a template nucleic acid, with each variant comprising a different nucleic acid sequence at a single codon position. Each variant nucleic acid encodes for a different amino acid at their single codon position, the different codons represented by X, Y, and Z.

FIGS. 6A-6E depict a reference amino acid sequence (FIG. 6A) having a number of amino acids, each residue indicated by a single circle, and variant amino acid sequences (FIGS. 6B, 6C, 6D, & 6E) generated using methods described herein. The reference amino acid sequence and variant sequences are encoded by nucleic acids and variants thereof generated by processes described herein.

FIGS. 7A-7B depict a reference amino acid sequence (FIG. 7A, SEQ ID NO: 24) and a library of variant amino acid sequences (FIG. 7B, SEQ ID NOS 25-31, respectively, in order of appearance), each variant comprising a single residue variant (indicated by an “X”). The reference amino acid sequence and variant sequences are encoded by nucleic acids and variants thereof generated by processes described herein.

FIGS. 8A-8B depict a reference amino acid sequence (FIG. 8A) and a library of variant amino acid sequences (FIG. 8B), each variant comprising two sites of single position variants. Each variant is indicated by differently patterned circles. The reference amino acid sequence and variant sequences are encoded by nucleic acids and variants thereof generated by processes described herein.

FIGS. 9A-9B depict a reference amino acid sequence (FIG. 9A) and a library of variant amino acid sequences (FIG. 9B), each variant comprising a stretch of amino acids (indicated by a box around the circles), each stretch having three sites of position variants (encoding for histidine) differing in sequence from the reference amino acid sequence. The reference amino acid sequence and variant sequences are encoded by nucleic acids and variants thereof generated by processes described herein.

FIGS. 10A-10B depict a reference amino acid sequence (FIG. 10A) and a library of variant amino acid sequences (FIG. 10B), each variant comprising two stretches of amino acid sequences (indicated by a box around the circles), each stretch having one site of single position variants (illustrated by the patterned circles) differing in sequence from reference amino acid sequence. The reference amino acid sequence and variant sequences are encoded by nucleic acids and variants thereof generated by processes described herein.

FIGS. 11A-11B depict a reference amino acid sequence (FIG. 11A) and a library of amino acid sequence variants (FIG. 11B), each variant comprising a stretch of amino acids (indicated by patterned circles), each stretch having a single site of multiple position variants differing in sequence from the reference amino acid sequence. In this illustration, 5 positions are varied where the first position has a 50/50 K/R ratio; the second position has a 50/25/25 V/L/S ratio, the third position has a 50/25/25 Y/R/D ratio, the fourth position has an equal ratio for all amino acids, and the fifth position has a 75/25 ratio for G/P. The reference amino acid sequence and variant sequences are encoded by nucleic acids and variants thereof generated by processes described herein.

FIG. 12 depicts a template nucleic acid encoding for an antibody having CDR1, CDR2, and CDR3 regions, where each CDR region comprises multiple sites for variation, each single site (indicated by a star) comprising a single position and/or stretch of multiple, consecutive positions interchangeable with any codon sequence different from the template nucleic acid sequence.

FIG. 13 depicts a plot of predicted variant distribution and resultant variant diversity.

FIG. 14 depicts an exemplary number of variants produced by interchanging sections of two expression cassettes (e.g., promotors, open reading frames, and terminators) to generate a variant library of expression cassettes.

FIG. 15 presents a diagram of steps demonstrating an exemplary process workflow for gene synthesis as disclosed herein.

FIG. 16 illustrates an example of a computer system.

FIG. 17 is a block diagram illustrating an architecture of a computer system.

FIG. 18 is a diagram demonstrating a network configured to incorporate a plurality of computer systems, a plurality of cell phones and personal data assistants, and Network Attached Storage (NAS).

FIG. 19 is a block diagram of a multiprocessor computer system using a shared virtual address memory space.

FIG. 20 depicts a BioAnalyzer plot of PCR reaction products resolved by gel electrophoresis.

FIG. 21 depicts an electropherogram showing 96 sets of PCR products, each set of PCR products differing in sequence from a wild-type template nucleic acid at a single codon position, where the single codon position of each set is located at a different site in the wild-type template nucleic acid sequence. Each set of PCR products comprises 19 variant nucleic acids, each variant encoding for a different amino acid at their single codon position.

FIG. 22 depicts a plot comparing observed frequency and expected probability of variants.

FIG. 23 depicts a plot of an average count per probability bin.

FIG. 24 depicts a plot of analysis of PCR products. X axis is base pairs and Y axis is fluorescent units.

FIG. 25 depicts a plot of distribution of observed combinatorial variants.

FIGS. 26A-26D illustrate generation of a non-saturating combinatorial library.

FIGS. 27A-27C depict schemas of variants in single or multiple CDR regions.

FIG. 28A depicts a schema of variants in single or multiple heavy chain and light chain scaffolds.

FIG. 28B depicts a schema of variants in a single or multiple frameworks.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

Provided herein are methods and compositions for production of synthetic (i.e. de novo synthesized or chemically synthesized) polynucleotides. The term oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA.

Provided herein are methods and compositions for production of synthetic (i.e. de novo synthesized) genes. Libraries comprising synthetic genes may be constructed by a variety of methods described in further detail elsewhere herein, such as PCA, non-PCA gene assembly methods or hierarchical gene assembly, combining (“stitching”) two or more double-stranded polynucleotides to produce larger DNA units (i.e., a chassis). Libraries of large constructs may involve polynucleotides that are at least 1, 1.5, 2, 3, 4, 5, 6, 7, 8, 9, 10, 15, 20, 30, 40, 50, 60, 70, 80, 90, 100, 125, 150, 175, 200, 250, 300, 400, 500 kb long or longer. The large constructs can be bounded by an independently selected upper limit of about 5000, 10000, 20000 or 50000 base pairs. The synthesis of any number of polypeptide-segment encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein, may comprise at least one region encoding for exon sequence(s) without an intervening intron sequence found in the corresponding genomic sequence. Alternatively, the corresponding genomic sequence to a cDNA may lack intron sequence in the first place.

Variant Library Synthesis

Methods described herein provide for synthesis of a library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library can be generated. Similarly, provided herein are methods for synthesis of variant libraries encoding for RNA sequences (e.g., miRNA, shRNA, and mRNA) or DNA sequences (e.g., enhancer, promoter, UTR, and terminator regions). In some instances, the sequences are exon sequences or coding sequences. In some instances, the sequences do not comprise intron sequences. Also provided herein are downstream applications for variants selected out of the libraries synthesized using methods described herein. Downstream applications include identification of variant nucleic acids or protein sequences with enhanced biologically relevant functions, e.g., biochemical affinity, enzymatic activity, changes in cellular activity, and for the treatment or prevention of a disease state.

Combinatorial Nucleic Acid Libraries

Described herein are methods for an efficient system of synthesizing variant nucleic acid libraries, which are highly accurate. Further provided herein are methods for synthesizing combination based variant libraries. An advantageous feature of methods provided herein is that the product and frequency of assembled nucleic acids in the combinatorial library can be accurately predicted, allowing for screening of the combinatorial library with an accurate understanding of those combinatorial products associated with a negative or null result, as well as those combinatorial products associated with an enhancement associated with a biochemical or cellular activity. Such a system is advantageous over contemporary methods, i.e. phage display, which do not allow for an efficient means to gather information on negative or null result. Another advantageous feature of methods provided herein is that when a representative combinatorial library is designed and tested, less material and associated costs are needed than in comparison to a fully saturating library, while also allowing for rapid generation of second and third generation libraries with a refined variegation criteria based on information gathered from screening of products of the first generation combinatorial library.

Methods as described herein for efficient and accurate synthesis of variant nucleic acid libraries may result in a uniform and diverse library. Libraries generated using methods described herein are non-random. Libraries generated using methods described herein provide for precise introduction of each intended variant at the desired frequency. Libraries generated using methods described herein provide for high precision on account of decreased dropout rate of representation and improved uniformity across species of polynucleotides or longer nucleic acids within each library. In addition, the benefits from such precision at the polynucleotide synthesis level allows for high precision at a functional level for the downstream applications, such as assessing protein activity from translation products incorporating predetermine variance encoded at the codon level. In some instances, methods as described herein for generation of precise libraries allows for an improved design of subsequent libraries. Such subsequent libraries may be more focused in the design as a result of information gathered on a negative or null result from a first library. For example, a first variant nucleic acid library synthesized using methods described herein may be used to generate a variant library of functional RNAs or proteins which are screened for a certain activity. Based on observations of both the positive and negative results associated with precisely defined, non-random libraries, design selections are then made for a second variant library which is then used for further screening steps for further screen and select for species associated with a specified activity. This process can be repeated 1, 2, 3, 4, 5, 6, 7, 8, 9, 10 or more times. Methods for library design, build, screening and repeating can be done to identify enhanced species associated with a single activity, or multiple activities (e.g., binding affinity, stability, and expression).

Using generation of libraries in silico, sequences may be known and be non-random. In some instances, the libraries comprise at least or about 10¹, 10², 10³, 10⁴, 10⁵, 10⁶, 10⁷, 10⁸, 10⁹, 10¹⁰, or more than 10¹⁰ variants. In some instances, sequences for each variant of the libraries comprising at least or about 10¹, 10², 10³, 10⁴, 10⁵, 10⁶, 10⁷, 10⁸, 10⁹, or 10¹⁰ variants are known. In some instances, the libraries comprise a predicted diversity of variants. In some instances, the diversity represented in the libraries is at least or about 60%, 65%, 70%, 75%, 80%, 85%, 90%, 95%, or more than 95% of the predicted diversity. In some instances, the diversity represented in the libraries is at least or about 70% of the predicted diversity. In some instances, the diversity represented in the libraries is at least or about 80% of the predicted diversity. In some instances, the diversity represented in the libraries is at least or about 90% of the predicted diversity. In some instances, the diversity represented in the libraries is at least or about 99% of the predicted diversity. As described herein the term “predicted diversity” refers to a total theoretical diversity in a population comprising all possible variants.

Generation of highly uniform and diverse libraries as described herein where sequences for each variant are known results in an accurate understanding of those combinatorial products associated with an enhanced or reduced activity and those combinatorial products associated with a negative or null result. Knowing the products associated with an enhanced or reduced activity and those combinatorial products associated with a negative or null result may allow for efficient use of the libraries for subsequent assays. For example, in performing a large screen, the variant sequences that will result in an enhanced or reduced activity are known. In performing subsequent screens, sequences that resulted in a negative or null result may be excluded such that only variant sequences that result in an enhanced or reduced activity are screened.

In some instances, the enhanced or reduced activity is associated with a cellular activity. The cellular activity includes, but is not limited to, reproduction, growth, adhesion, death, migration, energy production, oxygen utilization, metabolic activity, cell signaling, response to free radical damage, or any combination thereof.

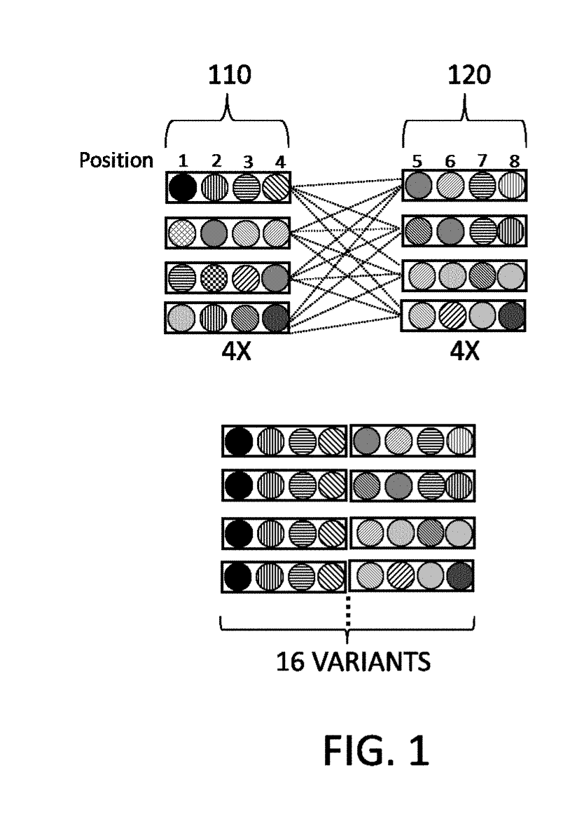

In a first exemplary process, a non-saturating combinatorial library is generated. Generation of a non-saturating combinatorial library can reduce the number of synthesis steps. Referring to FIG. 1, a first population of nucleic acids 110 exhibits diversity at positions 1, 2, 3, and 4. A second population of nucleic acids 120 exhibits diversity at positions 5, 6, 7, and 8. The first population of nucleic acids 110 is combined with the second population of nucleic acids 120 to yield 16 combinations of nucleic acid fragments. The first population of nucleic acids 110 can be combined with the second population of nucleic acids 120 by blunt end ligation. In some instances, the first population and the second population are designed such that they have a complementary overlapping sequence comprising a restriction enzyme recognition region, such that subsequent to cleavage of the nucleic acids in each population, the first population and the second population are able to anneal to each other.

In some cases, a nucleic acid library is synthesized with two or more nucleic acid fragments. A nucleic acid library can be synthesized with at least two fragments, at least 3 fragments, at least 4 fragments, at least 5 fragments, or more. The length of each of the nucleic acid fragments or average length of the nucleic acids synthesized may be at least or about at least 10, 15, 20, 25, 30, 35, 40, 45, 50, 100, 150, 200, 300, 400, 500, 2000 nucleotides, or more. The length of each of the nucleic acid fragments or average length of the nucleic acids synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the nucleic acid fragments or average length of the nucleic acids synthesized may fall from 10-2000, 10-500, 9-400, 11-300, 12-200, 13-150, 14-100, 15-50, 16-45, 17-40, 18-35, 19-25.

Various mixing processes, such as by ligation, and reagents are known in the art and can be useful for carrying out the methods provided herein. Blunt end ligation can be used to join a fragment from one population of nucleic acids with a fragment from a second population of nucleic acids. Ligases can include, but are not limited to, E. coli ligase, T4 ligase, mammalian ligases (e.g., DNA ligase I, DNA ligase II, DNA ligase III, DNA ligase IV), thermostable ligases, and fast ligases. In some instances, PCR extension overlap methods are used to anneal and link two fragments to form a longer nucleic acid. In such an arrangement, a first fragment has a region complementary to second fragment such that, in the presence of a DNA polymerase and amplification reagents, e.g., dNTPs, buffer solution, and ATP, each fragment serves as a primer for the other fragment for an amplification reaction extending from the location of annealing. In some instances, a fragment from one population of nucleic acids is joined with a fragment from a second population of nucleic acids by ligation subsequent to cleavage of a restriction enzyme recognition region. In some instances, the restriction enzyme generates overhangs that are then joined by a ligase. A molar ratio of 1:1 of one nucleic acid fragment to another nucleic acid fragment can be used. In some cases, the molar ratio is at least 1:1, at least 1:2, at least 1:3, at least 1:4, or more. Alternately, the ratio can be at least 2:1, at least 3:1, at least 4:1, or more. Total molar mass of the nucleic acid fragments ligated or the molar mass of each of the nucleic acid fragments may be at least or at least about 1, 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more.

In some cases, the nucleic acid fragments generated by the methods described herein are blunt ended prior to ligation. The nucleic acids can be blunted using T4 DNA polymerase or the Klenow fragment. Alternately, an enzyme (e.g., Sma I, Dpn I, Pvu II, Eco RV) is used that produces a blunt end directly. In some instances, a DNA endonuclease or a DNA exonuclease is used to produce blunt ends.

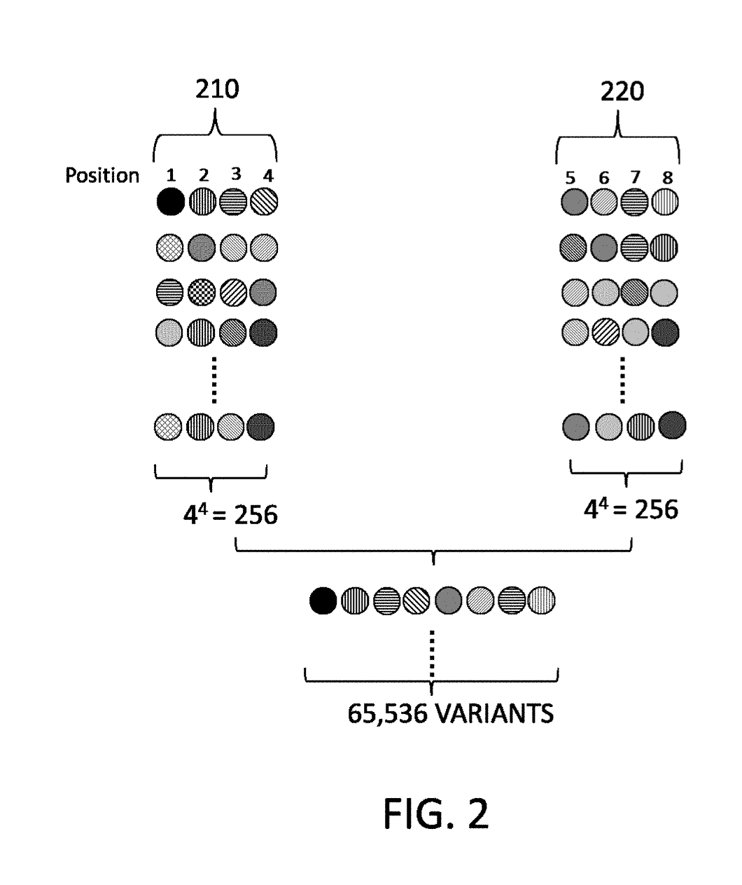

In a second exemplary workflow, a saturating combinatorial library is generated. Referring to FIG. 2, a first population of nucleic acids 210 exhibits diversity at positions 1, 2, 3, and 4. A second population of nucleic acids 220 exhibits diversity at positions 5, 6, 7, and 8. As seen in FIG. 2, the population of nucleic acids 210 on the “left” of the gene fragment has 4⁴ diversity. The population of nucleic acids 220 on the “right” of the gene fragment has 4⁴ diversity. A long gene fragment can then be synthesized with diversity across the “left” half of the desired gene combined with another fragment with diversity across the “right” half of the desired gene yielding 4⁸ total diversity. The length of each of the nucleic acid fragments or average length of the nucleic acids synthesized may be at least or about at least 10, 15, 20, 25, 30, 35, 40, 45, 50, 100, 150, 200, 300, 400, 500, 2000 nucleotides, or more. The length of each of the nucleic acid fragments or average length of the nucleic acids synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the nucleic acid fragments or average length of the nucleic acids synthesized may fall from 10-2000, 10-500, 9-400, 11-300, 12-200, 13-150, 14-100, 15-50, 16-45, 17-40, 18-35, 19-25.

The resulting nucleic acids can be verified. In some cases, the nucleic acids are verified by sequencing. In some instances, the nucleic acids are verified by high-throughput sequencing such as by next generation sequencing. Sequencing of the sequencing library can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

Provided herein are methods for the synthesis of nucleic acid libraries, non-saturating or saturating in their degree of variance, which are highly accurate. In some instances, about 70% of nucleic acids are insertion and deletion free. In some instances, at least 60%, 65%, 70%, 75%, 80%, 85%, 90%, 95%, 99%, or more than 99% of nucleic acids are insertion and deletion free. In some instances, about 60%, 65%, 70%, 75%, 80%, 85%, 90%, 95%, 99%, or more than 99% of nucleic acids are insertion and deletion free. In some instances, more than 90% of nucleic acids are insertion and deletion free. In some instance, at least 80% of the nucleic acids have no errors. In some instances, at least about 70%, 75%, 80%, 85%, 90%, 95%, 99%, or more of the nucleic acids have no errors.

Provided herein are methods for the synthesis of nucleic acid libraries, non-saturating or saturating in their degree of variance, which are highly accurate. In some instances, more than 80% of nucleic acids in a de novo synthesized nucleic acid library described herein are represented within at least about 1.5× the mean representation for the entire library following amplification. In some instances, more than 80% of nucleic acids in a de novo synthesized nucleic acid library described herein are represented within at least about 1.5×, 2×, 2.5×, 3×, 3.5×, or 4× the mean representation for the entire library following amplification. In some instances, more than 90% of nucleic acids in a de novo synthesized nucleic acid library described herein are represented within at least about 1.5× the mean representation for the entire library following amplification. In some instances, more than 90% of nucleic acids in a de novo synthesized nucleic acid library described herein are represented within at least about 1.5×, 2×, 2.5×, 3×, 3.5×, or 4× the mean representation for the entire library following amplification. In some instances, more than 80% of nucleic acids in a de novo synthesized nucleic acid library described herein are represented within at least about 2× the mean representation for the entire library following amplification. In some instances, more than 80% of nucleic acids in a de novo synthesized nucleic acid library described herein are represented within at least about 2× the mean representation for the entire library following amplification.

Generation of Representative Nucleic Acid Libraries

Described herein are methods for synthesizing nucleic acid libraries having a preselected distribution of variant codon encoding regions. Moreover, such library may be non-saturating for the preselected distribution while providing insight into a representative distribution. Further provided herein are methods relating to generation of nucleic acids that, once translated, provide for a preselected distribution of amino acids at a specific position. By generating random samples from the preselected distribution, a less than saturating nucleic acid library is designed to have a representative distribution close to the preselected population distribution. Nucleic acid libraries as described herein with representative distribution close to the preselected population distribution may further comprise precise introduction of each intended variant at the desired preselected distribution.

Computational techniques described herein include, without limitation, random sampling. In a first process, for a preselected distribution of codon variance at each position, a cumulative distribution value for each position is calculated. In some instances, the cumulative distribution value maps to a probability between about 0.0 and 1.0. For a population of nucleic acids, the cumulative distribution value provides for determining the likelihood of a codon variant at a particular position. For example, the number of times at each position the codon variant appears across the population of nucleic acids is summed, and the percentage that each amino acid appears at each position can then be determined. The percentage in the sample population of nucleic acids is then compared to the preselected distribution. With a sufficient number of nucleic acids in a population, a sample distribution is generated that aligns with the preselected distribution. In some instances, the sampling performed is a form of Monte Carlo sampling, applying uniform random sampling.

In some instances, nucleic acid libraries designed and synthesized to have a preselected distribution encode for about 1%, 5%, 10%, 15%, 20%, 25%, 30%, 35%, 40%, 45%, 50%, 55%, 60%, or more than 60% of non-identical nucleic acids compared to a saturating nucleic acid library. In some instances, nucleic acid libraries designed and synthesized to have a preselected distribution encode for at least 1%, 5%, 10%, 15%, 20%, 25%, 30%, 35%, 40%, 45%, 50%, 55%, 60%, or more than 60% of non-identical nucleic acids compared to a saturating nucleic acid library.

In some instances, nucleic acid libraries designed and synthesized to have a preselected distribution encode for about 1%, 5%, 10%, 15%, 20%, 25%, 30%, 35%, 40%, 45%, 50%, 55%, 60%, or more than 60% of non-identical nucleic acids compared to a larger nucleic acid library. In some instances, nucleic acid libraries designed and synthesized to have a preselected distribution encode for at least 1%, 5%, 10%, 15%, 20%, 25%, 30%, 35%, 40%, 45%, 50%, 55%, 60%, or more than 60% of non-identical nucleic acids compared to a larger nucleic acid library.

In some instances, the number of nucleic acids designed and synthesized in a representative sub-population from a larger variant nucleic acid library is in the range of about 50-100000, 100-75000, 250-50000, 500-25000, and 1000-15000, 2000-10000, and 4000-8000 sequences. In some instances, a population of nucleic acids is 500 sequences. In some instances, a population of nucleic acids is 5000, 10000, or 15000 sequences. In some instances, a population of nucleic acids has at least 50, 100, 150, 500, 1000, 2000, 5000, 10000, 20000, 50000, 100000, 200000, 400000, 800000, 1000000, or more different sequences. In some instances, each population of nucleic acids is up to 50, 100, 500, 1000, 2000, 5000, 10000, 20000, 50000, 100000, 200000, 400000, 800000, or 1000000.

In some instances, synthesis of nucleic acid libraries by combinatorial methods to arrive at a preselected distribution of variant codon encoding regions represents 70% to 99% of the predicted diversity. In some instances, synthesis of nucleic acid libraries by combinatorial methods to arrive at a preselected distribution of variant codon encoding regions represent at least 70% of the predicted diversity. In some instances, synthesis of nucleic acid libraries by combinatorial methods to arrive at a preselected distribution of variant codon encoding regions represent 70% to 75%, 70% to 80%, 70% to 85%, 70% to 90%, 70% to 95%, 70% to 97%, 70% to 99%, 75% to 80%, 75% to 85%, 75% to 90%, 75% to 95%, 75% to 97%, 75% to 99%, 80% to 85%, 80% to 90%, 80% to 95%, 80% to 97%, 80% to 99%, 85% to 90%, 85% to 95%, 85% to 97%, 85% to 99%, 90% to 95%, 90% to 97%, 90% to 99%, 95% to 97%, 95% to 99%, or 97% to 99% of the predicted diversity. In some instances, the diversity represented of the synthesized representative population of nucleic acids is at least or about 60%, 65%, 70%, 75%, 80%, 85%, 90%, 95%, or more than 95% of the predicted diversity. In some instances, the diversity represented of the synthesized representative population of nucleic acids is 99% of the predicted diversity.

Generation of Representative Nucleic Acid Libraries Using Combinatorial Methods

Provided herein are methods for synthesis of nucleic acid libraries by combinatorial methods to arrive at a preselected distribution of variant codon encoding regions. In some instances, a reference sequence, serving as the template for variant for synthesizing a population of nucleic acids, is split such that a first portion is a reference sequence for a first variant population of nucleic acids, and a second portion is a reference sequence for a second variant population of nucleic acids.

In some instances, random sampling methods as described herein are used to generate a representative variant distribution for portions from a larger variant library. A first representative population of nucleic acids, representing variants for a first portion of a full reference sequence, and a second representative population of nucleic acids, representing variant for a second portion of a full reference sequence are synthesized and then combined by ligation, such as by blunt end ligation or by some other biochemical technique known in the art. In some cases, a resulting nucleic acid library is saturating. In some cases, a resulting nucleic acid library is non-saturating.

In some cases, a nucleic acid library is synthesized with two or more variant nucleic acid populations that, when joined, results in a desired longer nucleic acid variant library. A nucleic acid library can be synthesized with at least 2, 3, 4, 5, 6, 7, 8, 9, 10, or more than 10 populations, each encoding for a different region of a reference nucleic acid. In some instances, each nucleic acid population is in the range of about 50-100000, 100-75000, 250-50000, 500-25000, and 1000-15000, 2000-10000, and 4000-8000 sequences. In some instances, each nucleic acid population is about 500, 1000, 5000, 10000, 15000 or more sequences. In some instance, each nucleic acid population is at least 50, 100, 150, 500, 1000, 2000, 5000, 10000, 20000, 50000, 100000, 200000, 400000, 800000, 1000000, or more. In some instance, each nucleic acid population is up to 50, 100, 500, 1000, 2000, 5000, 10000, 20000, 50000, 100000, 200000, 400000, 800000, and 1000000.

In some instances, synthesis of nucleic acid libraries by combinatorial methods to arrive at a preselected distribution of variant codon encoding regions represent 70% to 99% of the predicted diversity. In some instances, synthesis of nucleic acid libraries by combinatorial methods to arrive at a preselected distribution of variant codon encoding regions represent at least 70% of the predicted diversity. In some instances, synthesis of nucleic acid libraries by combinatorial methods to arrive at a preselected distribution of variant codon encoding regions represent 70% to 75%, 70% to 80%, 70% to 85%, 70% to 90%, 70% to 95%, 70% to 97%, 70% to 99%, 75% to 80%, 75% to 85%, 75% to 90%, 75% to 95%, 75% to 97%, 75% to 99%, 80% to 85%, 80% to 90%, 80% to 95%, 80% to 97%, 80% to 99%, 85% to 90%, 85% to 95%, 85% to 97%, 85% to 99%, 90% to 95%, 90% to 97%, 90% to 99%, 95% to 97%, 95% to 99%, or 97% to 99% of the predicted diversity. In some instances, synthesis of nucleic acid libraries by combinatorial methods to arrive at a preselected distribution of variant codon encoding regions is at least or about 60%, 65%, 70%, 75%, 80%, 85%, 90%, 95%, or more than 95% of the predicted diversity. In some instances, the diversity represented of the synthesized representative population of nucleic acids is 99% of the predicted diversity.

Synthesis Followed by PCR Mutagenesis

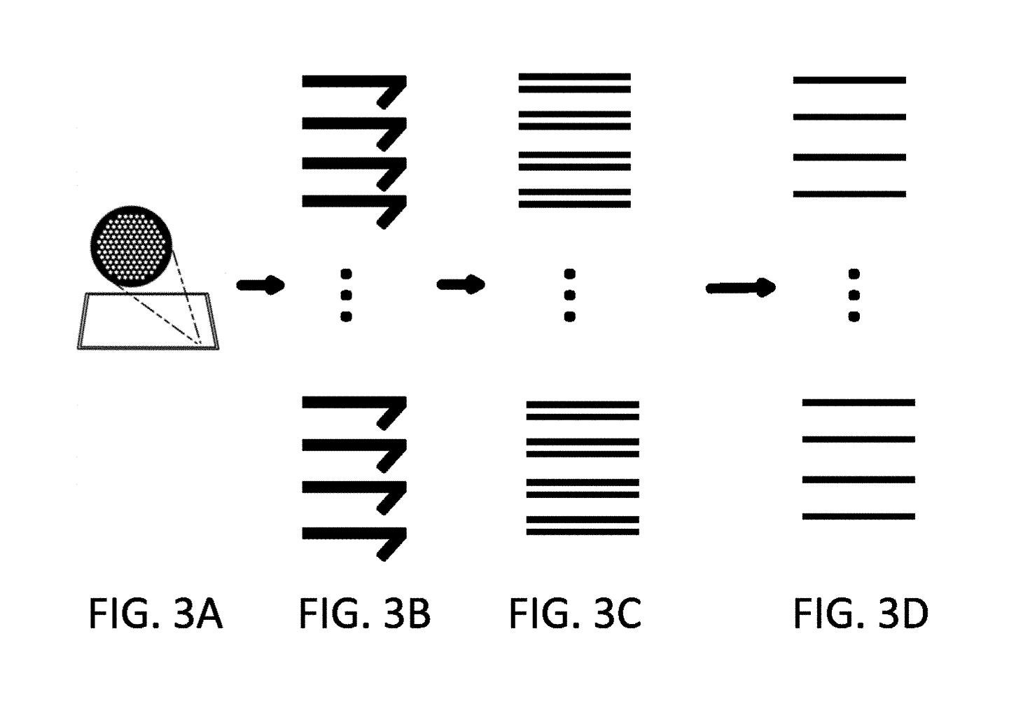

Nucleic acid libraries generated by combinatorial methods described herein (e.g. saturating or non-saturating) can be used for PCR mutagenesis methods. In some cases, the representative nucleic acid library having a preselected distribution is used for PCR mutagenesis methods. In this workflow, a plurality of polynucleotides are synthesized, wherein each polynucleotide encodes for a predetermined sequence which is a predetermined variant of a reference nucleic acid sequence. Referring to the figures, an exemplary workflow in depicted in FIGS. 3A-3D, wherein polynucleotides are generated on a surface. FIG. 3A depicts an expansion view of a single cluster of a surface with 121 loci. Each nucleic acid depicted in FIG. 3B is a primer that can be used for amplification from a reference nucleic acid sequence to produce a library of variant long nucleic acids, FIG. 3C. The library of variant long nucleic acids is then, optionally, subject to transcription and or translation to generate a variant RNA or protein library, FIG. 3D. In this exemplary illustration, a device having a substantially planar surface is used for de novo synthesis of polynucleotides is depicted, FIG. 3A. In some instances, the device comprises a cluster of loci, wherein each locus is a site for polynucleotide extension. In some instances, a single cluster comprises all the polynucleotide variants needed to generate a desired variant sequence library. In an alternative arrangement, a plate comprises a field of loci which are not segregated into clusters.

Provided herein are methods for synthesis of polynucleotides within a cluster (e.g., as seen in FIG. 3) followed by amplification of polynucleotides within a single cluster. Such an arrangement provides for improved nucleic acid representation in comparison to amplification of non-identical polynucleotides across an entire plate without a clustered arrangement. In some instances, amplification of polynucleotides synthesized on surfaces of loci within a cluster overcomes negative effects on representation due to repeated synthesis of large polynucleotide populations having polynucleotides with heavy GC content. In some instances, a cluster described herein, comprises about 50-1000, 75-900, 100-800, 125-700, 150-600, 200-500, or 300-400 discrete loci. In some instances, a loci is a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence. In some instances, 1× redundancy means having no polynucleotides with identical sequence.

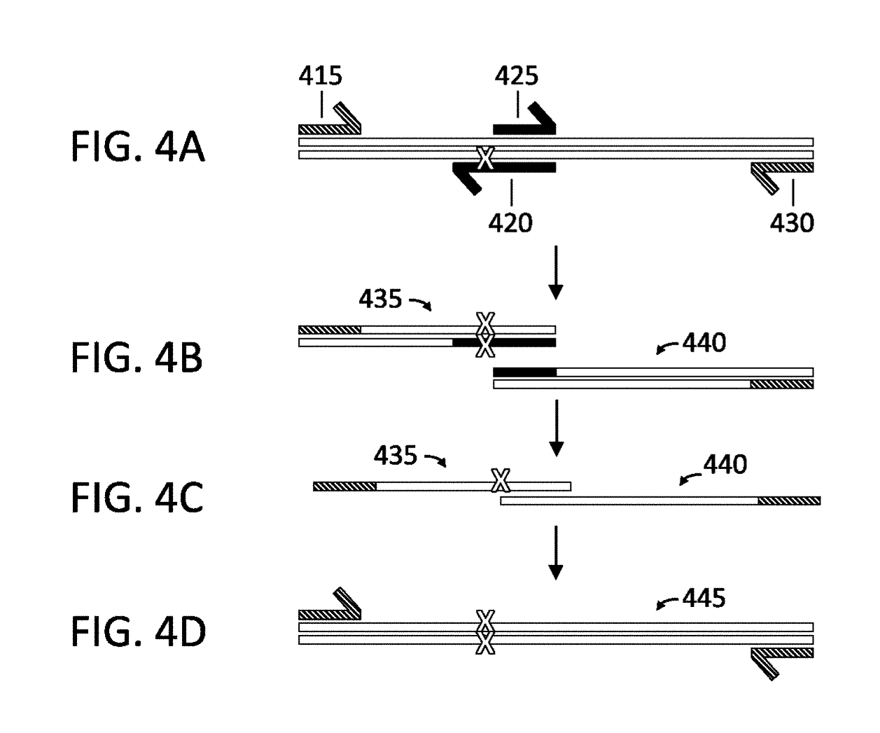

A de novo synthesized polynucleotide library described herein may comprise a plurality of polynucleotides, each with at least one variant sequence at first position, position “x”, and each variant polynucleotide is used as a primer in a first round of PCR to generate a first extension product. In this example, position “x” in a first polynucleotide 420 encodes for a variant codon sequence, i.e., one of 19 possible variants from a reference sequence. See FIG. 4A. A second polynucleotide 425 comprising a sequence overlapping that of the first polynucleotide is also used as a primer in a separate round of PCR to generate a second extension product. In addition, outer primers 415, 430 may be used for amplification of fragments from a long nucleic acid sequence. The resultant amplification products are fragments of the long nucleic acid sequence 435, 440. See FIG. 4B. The fragments of the long nucleic acid sequence 435, 440 are then hybridized, and subject to an extension reaction to form a variant of the long nucleic acid 445. See FIG. 4C. The overlapping ends of the first and second extension products may serve as primer of a second round of PCR, thereby generating a third extension product (FIG. 4D) that contains the variant. To increase the yield, the variant of the long nucleic acid is amplified in a reaction including a DNA polymerase, amplification reagents, and the outer primers 415, 430. In some instances, the second polynucleotide comprises a sequence adjacent to, but not including, the variant site. In an alternative arrangement, a first polynucleotide is generated that has a region that overlaps with a second polynucleotide. In this scenario, the first nucleic acid is synthesized with variation at a single codon for up to 19 variants. The second nucleic acid does not comprise a variant sequence. Optionally, a first population comprises the first polynucleotide variants and additional polynucleotides encoding for variants at a different codon site. Alternatively, the first polynucleotide and the second polynucleotide may be designed for blunt end ligation.

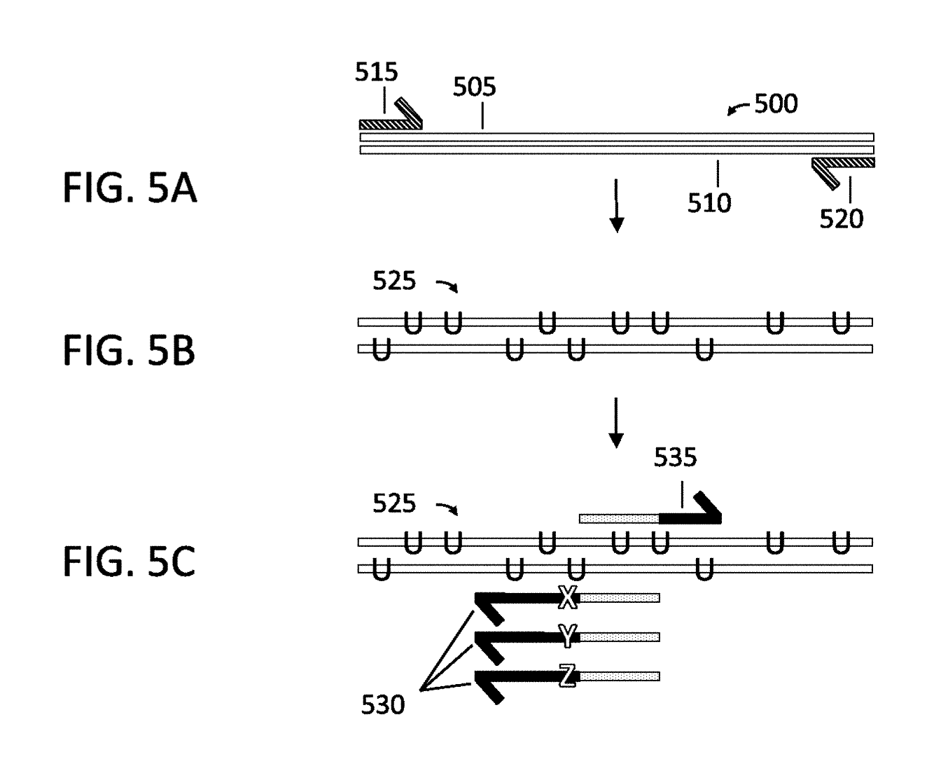

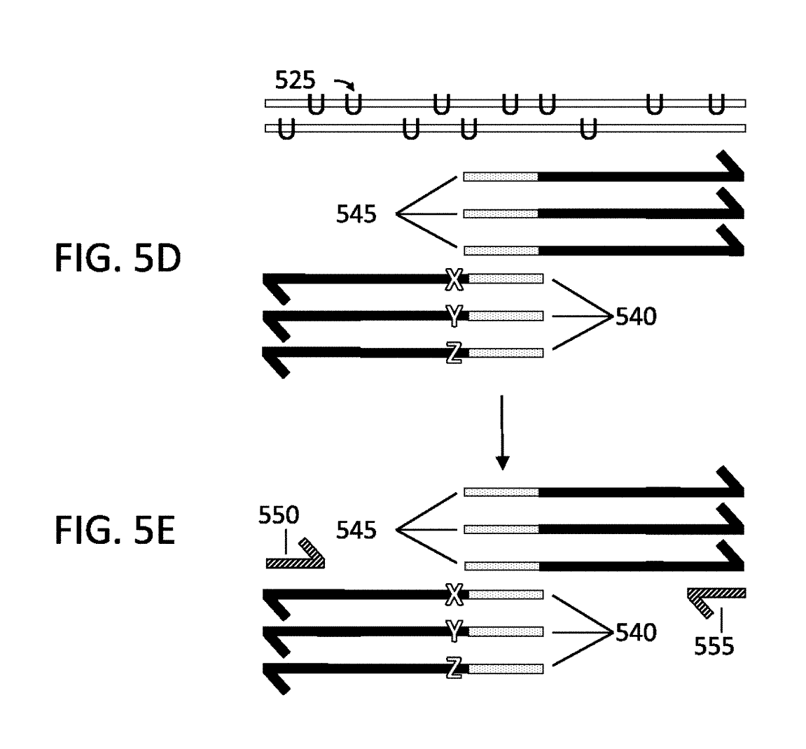

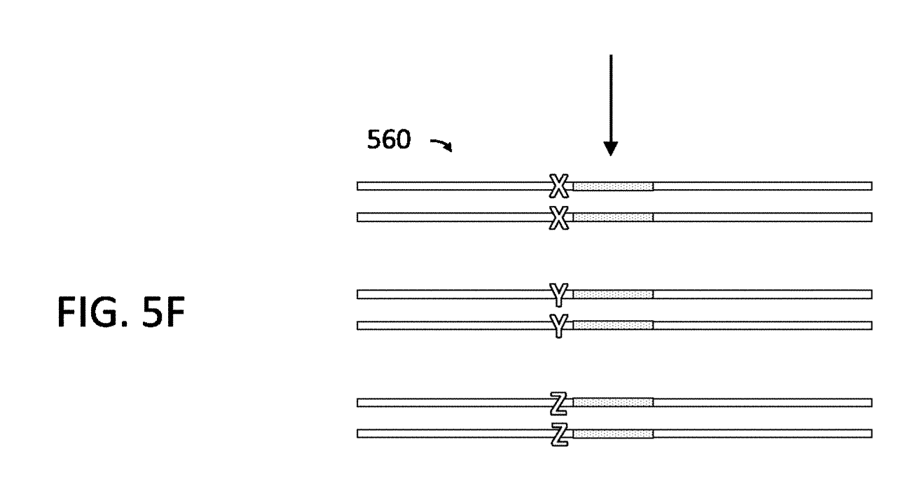

An alternative mutagenesis PCR method is depicted in FIGS. 5A-5F. In such a process, a template nucleic acid molecule 500 comprising a first and second strand 505, 510 is amplified in a PCR reaction containing a first primer 515 and a second primer 520 (FIG. 5A). The amplification reaction includes uracil as a nucleotide reagent. A uracil-labeled extension product 525 (FIG. 5B) is generated, optionally purified, and serves as a template for a subsequent PCR reaction using a first polynucleotide 535 and a plurality of second polynucleotides 530 to generate first extension products 540 and 545 (FIGS. 5C-5D). In this process, a plurality of polynucleotides 530 comprises polynucleotides encoding for variant sequences (denoted as X, Y, and Z, in FIG. 5C). The uracil-labeled template nucleic acid is digested by a uracil-specific excision reagent, e.g., USER digest available commercially from New England Biolabs. Variant 535 and different codons 530 with variants X, Y, and Z are added and a limited PCR step is performed to generate FIG. 5D. After the uracil-containing template is digested, the overlapping ends of the extension products serve to prime a PCR reaction with the first extension products 540 and 545 acting as primers in combination with a first outer primer 550 and a second outer primer 555, thereby generating a library of nucleic acid molecules 560 containing a plurality of variants X, Y, and Z at the variant site FIG. 5F.

De Novo Synthesis of a Population with Variant and Non-Variant Portions of a Long Nucleic Acid

Nucleic acid libraries generated by combinatorial methods described herein (e.g. saturating or non-saturating) can be used for de novo synthesis of multiple fragments of a long nucleic acid, wherein at least one of the fragments is synthesized in multiple versions, each version being of a different variant sequence. In some cases, the representative nucleic acid library having a preselected distribution is used for de novo synthesis, wherein at least one of the fragments is synthesized in multiple versions, each version being of a different variant sequence. In this arrangement, all of the fragments needed to assemble a library of variant long range nucleic acids are de novo synthesized. The synthesized fragments may have an overlapping sequence such that, following synthesis, the fragment library is subject to hybridization. Following hybridization, an extension reaction may be performed to fill in any complementary gaps.

Alternatively, the synthesized fragments may be amplified with primers and then subject to either blunt end ligation or overlapping hybridization. In some instances, the device comprises a cluster of loci, wherein each locus is a site for polynucleotide extension. In some instances, a single cluster comprises all the polynucleotide variants and other fragment sequences of a predetermined long nucleic acid to generate a desired variant nucleic acid sequence library. The cluster may comprise about 50 to 500 loci. In some arrangements, a cluster comprises greater than 500 loci.

Each individual polynucleotide in the first polynucleotide population may be generated on a separate, individually addressable locus of a cluster. One polynucleotide variant may be represented by a plurality of individually addressable loci. Each variant in the first polynucleotide population may be represented 1, 2, 3, 4, 5, 6, 7, 8, 9, 10, or more times. In some instances, each variant in the first polynucleotide population is represented at 3 or less loci. In some instances, each variant in the first polynucleotide population is represented at two loci. In some instances, each variant in the first polynucleotide population is represented at only a single locus.

Methods are provided herein to generate nucleic acid libraries with reduced redundancy. In some instances, variant nucleic acids may be generated without the need to synthesize the variant nucleic acid more than 1 time to obtain the desired variant nucleic acid. In some instances, the present disclosure provides methods to generate variant nucleic acids without the need to synthesize the variant nucleic acid more than 1, 2, 3, 4, 5 times, 6, 7, 8, 9, 10, or more times to generate the desired variant nucleic acid.

Variant nucleic acids may be generated without the need to synthesize the variant nucleic acid at more than 1 discrete site to obtain the desired variant nucleic acid. The present disclosure provides methods to generate variant nucleic acids without the need to synthesize the variant nucleic acid at more than 1 site, 2 sites, 3 sites, 4 sites, 5 sites, 6 sites, 7 sites, 8 sites, 9 sites, or 10 sites, to generate the desired variant nucleic acid. In some instances, a nucleic acid is synthesized in at most 6, 5, 4, 3, 2, or 1 discrete sites. The same nucleic acid may be synthesized in 1, 2, or 3 discrete loci on a surface.

In some instances, the amount of loci representing a single variant nucleic acid is a function of the amount of nucleic acid material required for downstream processing, e.g., an amplification reaction or cellular assay. In some instances, the amount of loci representing a single variant nucleic acid is a function of the available loci in a single cluster.

Provided herein are methods for generation of a library of nucleic acids comprising variant nucleic acids differing at a plurality of sites in a reference nucleic acid. In such cases, each variant library is generated on an individually addressable locus within a cluster of loci. It will be understood that the number of variant sites represented by the nucleic acid library will be determined by the number of individually addressable loci in the cluster and the number of desired variants at each site. In some instances, each cluster comprises about 50 to 500 loci. In some instances, each cluster comprises 100 to 150 loci.

In an exemplary arrangement, 19 variants are represented at a variant site corresponding to codons encoding for each of the 19 possible variant amino acids. In another exemplary case, 61 variants are represented at a variant site corresponding to triplets encoding for each of the 19 possible variant amino acids. In a non-limiting example, a cluster comprises 121 individually addressable loci. In this example, a nucleic acid population comprises 6 replicates each of a single-site variant (6 replicates×1 variant site×19 variants=114 loci), 3 replicates each of a double-site variant (3 replicates×2 variant sites×19 variants=114 loci), or 2 replicates each of a triple-site variant (2 replicates×3 variant sites×19 variants=114 loci). In some instances, a nucleic acid population comprises variants at four, five, six or more than six variant sites.

Provided herein are methods and compositions for production of synthetic (i.e. de novo synthesized or chemically synthesized) nucleic acids. Libraries of synthesized nucleic acids described herein may comprise a plurality of nucleic acids collectively encoding for one or more genes or gene fragments. In some instances, the nucleic acid library comprises coding or non-coding sequence. In some instances, the nucleic acid library encodes for a plurality of cDNA sequences. In some instances, the nucleic acid library comprises one or more nucleic acids, each of the one or more nucleic acids encoding sequence for multiple exons. Each nucleic acid within a library described herein may encode a different sequence, i.e., non-identical sequence. In some instances, each nucleic acid within a library described herein comprises at least one portion that is complementary to sequence of another nucleic acid within the library. Nucleic acid sequences described herein may be, unless stated otherwise, comprise DNA or RNA.

Provided herein are methods and compositions for production of synthetic (i.e. de novo synthesized) genes. Libraries comprising synthetic genes may be constructed by a variety of methods described in further detail elsewhere herein, such as PCA, non-PCA gene assembly methods or hierarchical gene assembly, combining (“stitching”) two or more double-stranded nucleic acids to produce larger DNA units (i.e., a chassis). Libraries of large constructs may involve nucleic acids that are at least 1, 1.5, 2, 3, 4, 5, 6, 7, 8, 9, 10, 15, 20, 30, 40, 50, 60, 70, 80, 90, 100, 125, 150, 175, 200, 250, 300, 400, 500 kb long or longer. The large constructs may be bound by an independently selected upper limit of about 5000, 10000, 20000 or 50000 base pairs. The synthesis of any number of polypeptide-segment encoding nucleotide sequences may include sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of nucleic acids: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. In the context of cDNA, the term gene or gene fragment refers to a DNA nucleic acid sequence comprising at least one region encoding for exon sequences without an intervening intron sequence.

In various embodiments, methods and compositions described herein relate to a library of genes. The gene library may comprise a plurality of subsegments. In one or more subsegments, the genes of the library may be covalently linked together. In one or more subsegments, the genes of the library may encode for components of a first metabolic pathway with one or more metabolic end products. In one or more subsegments, genes of the library may be selected based on the manufacturing process of one or more targeted metabolic end products. The one or more metabolic end products may comprise a biofuel. In one or more subsegments, the genes of the library may encode for components of a second metabolic pathway with one or more metabolic end products. The one or more end products of the first and second metabolic pathways may comprise one or more shared end products. In some cases, the first metabolic pathway comprises an end product that is manipulated in the second metabolic pathway.

Variant Nucleic Acid Libraries for an Organism

Variant nucleic acid libraries generated by methods described herein may encode for at least one gene of an organism. In some cases, the nucleic acid libraries encode for a single gene, a pathway or an entire genome of an organism. In some instances, the variant nucleic acid library encodes at least one of a gene (e.g., 1000 base pairs), parts (e.g., 3-10 genes), pathways (e.g., 10-100 genes), or a chassis (e.g., 100-1000 genes) of an organism. A non-limiting exemplary list of model organisms is provided in Table 1.

TABLE 1
Model Organism and Gene Number

| Model Organism | Protein Coding Genes* |
|---|---|
| Arabidopsis thaliana | 27000 |
| Caenorhabditis elegans | 20000 |
| Canis lupus familiaris | 19000 |
| Chlamydomonas reinhardtii | 14000 |
| Danio rerio | 26000 |
| Dictyostelium discoideum | 13000 |
| Drosophila melanogaster | 14000 |
| Escherichia coli | 4300 |
| Macaca mulatta | 22000 |
| Mus musculus | 20000 |
| Oryctolagus cuniculus | 27000 |
| Rattus norvegicus | 22000 |
| Saccharomyces cerevisiae | 6600 |
| Sus scrofa | 21000 |

*Numbers here reflect the number of protein coding genes and excludes tRNA and non-coding RNA. Ron Milo & Rob Phillips, Cell Biology by the Numbers 286 (2015).

Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 2 provides a listing of each codon possible (and the representative amino acid) for a variant site.

TABLE 2
List of Codons and Amino Acids

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Iso | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |
| Threonine | T | Thr | ACA | ACC | ACG | ACT | | |
| Valine | V | Val | GTA | GTC | GTG | GTT | | |
| Tryptophan | W | Trp | TGG | | | | | |
| Tyrosine | Y | Tyr | TAC | TAT | | | | |

Provided herein are variant nucleic acid libraries comprising nucleic acids that encode for a variant codon sequence compared to a reference nucleic acid sequence, wherein the variant codon sequence is chosen based on a codon assignment. An exemplary codon assignment is seen in Table 3 in which a variant codon sequence is chosen first from left to right. In some instances, the codon assignment is based on frequency of a codon in an organism. Exemplary organisms include, but are not limited, to an animal, plant, fungus, protist, archaeon, or bacterium. For example, the codon assignment is based on Escherichia coli or Homo sapiens.

TABLE 3
Codon Assignment

| | One | Three | | | | | | |
|---|---|---|---|---|---|---|---|---|
| | letter | letter | | | | | | |
| Amino Acids | code | code | Codons | | | | | |
| Alanine | A | Ala | GCT | GCA | GCC | GCG | | |
| Cysteine | C | Cys | TGC | TGT | | | | |
| Aspartic acid | D | Asp | GAT | GAC | | | | |
| Glutamic acid | E | Glu | GAG | GAA | | | | |
| Phenylalanine | F | Phe | TTC | TTT | | | | |
| Glycine | G | Gly | GGT | GGA | GGC | GGG | | |
| Histidine | H | His | CAC | CAT | | | | |
| Isoleucine | I | Iso | ATC | ATT | ATA | | | |
| Lysine | K | Lys | AAG | AAA | | | | |
| Leucine | L | Leu | CTG | CTC | CTT | TTG | TTA | CTA |
| Methionine | M | Met | ATG | | | | | |
| Asparagine | N | Asn | AAC | AAT | | | | |
| Proline | P | Pro | CCT | CCA | CCG | CCC | | |
| Glutamine | Q | Gln | CAG | CAA | | | | |
| Arginine | R | Arg | AGA | CGT | AGG | CGA | CGC | CGG |
| Serine | S | Ser | AGC | TCT | TCC | AGT | TCA | TCG |
| Threonine | T | Thr | ACC | ACA | ACT | ACG | | |
| Valine | V | Val | GTG | GTT | GTC | GTA | | |
| Tryptophan | W | Trp | TGG | | | | | |
| Tyrosine | Y | Tyr | TAC | TAT | | | | |
| Stop codon | | | TGA | TAA | TAG | | | |

Provided herein are variant nucleic acid libraries comprising nucleic acids that encode for a variant codon sequence compared to a reference nucleic acid sequence, wherein the variant codon sequence based on the codon assignment is determined by various factors. In some instances, the variant codon sequence is chosen based on complexity or diversity of the codon sequence. For example, a codon sequence comprising three different nucleobases is chosen instead of a codon sequence comprising two different nucleobases or a codon sequence comprising same nucleobases. In some instances, the codon sequence is chosen based on downstream applications. Downstream applications include, but are not limited to, minimizing effects on expression levels following protein translation or improving detection of the variant codon sequence by next generation sequencing. Improving detection of the variant codon sequence by next generation sequencing may comprise avoiding homopolymers with high error rates. In some instances, the codon sequence is chosen unless the codon sequence results in a site that results in a disruption in the sequence such as a restriction enzyme site.

Codon sequences for a variant site based on a codon assignment as described herein may be randomized. In some instances, the codon sequence is not randomized. For example, for single variant libraries where one mutation is chosen per peptide, the codon sequences are not randomized. In some instances, multiple variant libraries comprise codon sequences that are randomized.

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100, 125, 150, 175, 200, 225, 250, 275, 300, or more codons in a single long nucleic acid.

Provided herein are processes where a second nucleic acid population is generated on a second cluster containing a plurality of individually addressable loci. The second nucleic acid population may comprise a plurality of second nucleic acids that are constant for each codon position (i.e., encode the same amino acid at each position). The second nucleic acid may overlap with at least a portion of the first nucleic acids. In some instances, the second nucleic acids do not contain the variant site represented on the first nucleic acids. Alternatively, the second nucleic acid population may comprise a plurality of second nucleic acids that contain at least one variant for one or more codon positions.

Provided herein are methods for synthesizing a library of nucleic acids where a single population of nucleic acids is generated comprising variants at multiple codon positions. A first nucleic acid population may be generated on a first cluster containing a plurality of individually addressable loci. In such cases, the first nucleic acid population comprises variants at different codon positions. In some instances, the different sites are consecutive (i.e., encoding consecutive amino acids). For example, the first nucleic acid population comprises variants in two consecutive codon positions, encoding up to 19 variants at a position. In some instances, the first nucleic acid population comprises variants in two consecutive codon positions, encoding from about 1 to about 19 variants at a position. In some instances, about 38 nucleic acids are synthesized. A first nucleic acid population may comprise varied nucleic acids collectively encoding up to 19 codon variants at the same, or additional variant site. A first nucleic acid population may include a plurality of first nucleic acids that contains up to 19 variants at position x, up to 19 variants at position y, and up to 19 variants at position z. In such an arrangement, each variant encodes a different amino acid such that up to 19 amino acid variants are encoded at each of the different variant sites. In an additional instance, a second nucleic acid population is generated on a second cluster containing a plurality of individually addressable loci. The second nucleic acid population may comprise a plurality of second nucleic acids that are constant for each codon position (i.e., encode the same amino acid at each position). The second nucleic acids may overlap with at least a portion of the first nucleic acids. The second nucleic acids may not contain the variant site represented on the first nucleic acids.

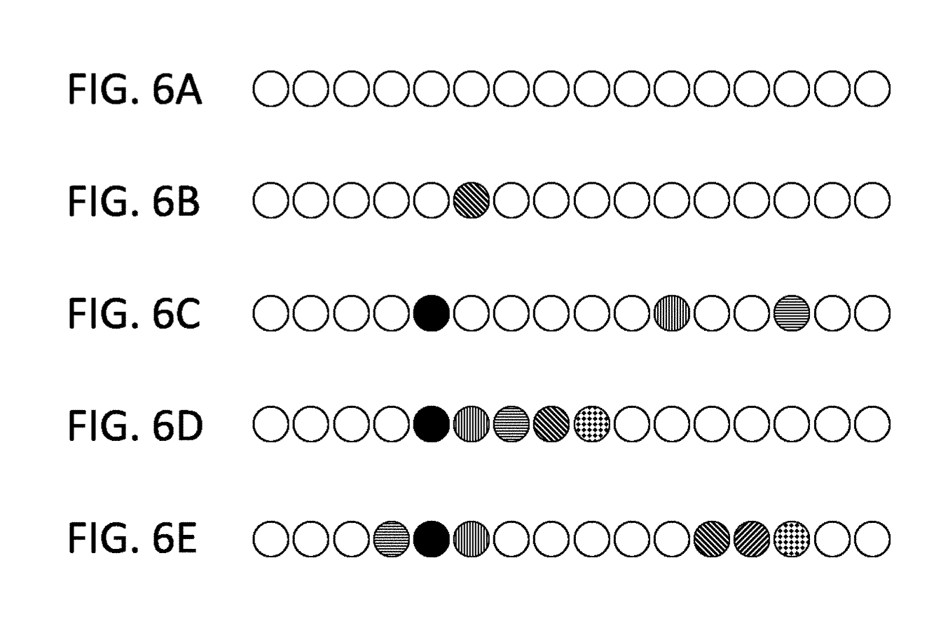

Variant nucleic acid libraries generated by processes described herein provide for the generation of variant protein libraries. In a first exemplary arrangement, a template nucleic acid encodes for sequence that, when transcribed and translated, results in a reference amino acid sequence (FIG. 6A) having a number of codon positions, indicated by a single circle. Nucleic acid variants of the template can be generated using methods described herein. In some instances, a single variant is present in the nucleic acid, resulting in a single amino acid sequence (FIG. 6B). In some instances, more than one variant is present in the nucleic acid, wherein the variants are separated by one or more codons, resulting in a protein with spacing between variant residues (FIG. 6C). In some instances, more than one variant is present in the nucleic acid, wherein the variants are sequential and adjacent or consecutive to one another, resulting in spaced variant stretches of residues (FIG. 6D). In some instances, two stretches of variants are present in the nucleic acid, wherein each stretch of variants comprises sequential and adjacent or consecutive variants (FIG. 6E).

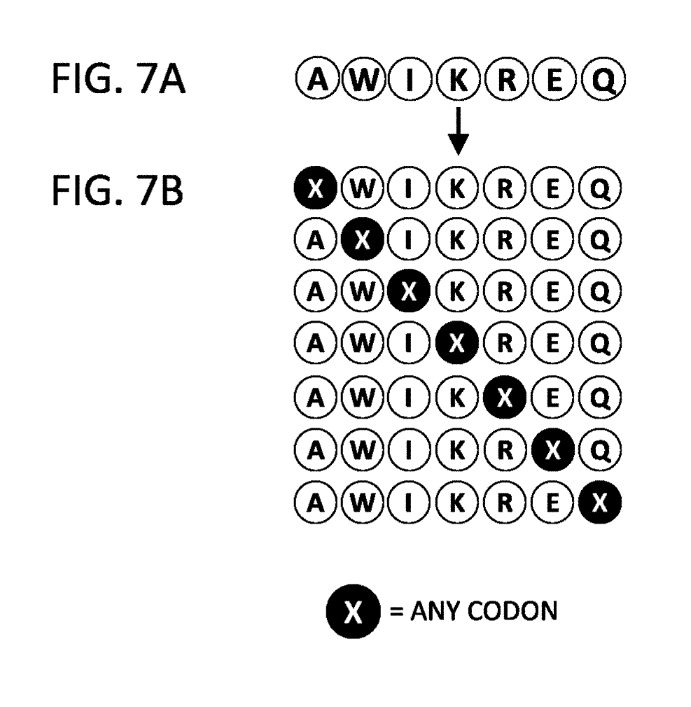

Provided herein are methods to generate a library of nucleic acid variants, wherein each variant comprises a single position codon variant. In one instance, a template nucleic acid has a number of codon positions wherein exemplary amino acid residues are indicated by circles with their respective one letter code protein codon, FIG. 7A. FIG. 7B depicts a library of amino acid variants encoded by a library of variant nuclei acids, wherein each variant comprises a single position variant, indicated by an “X”, located at a different single site. A first position variant has any codon to replace alanine, a second variant with any codon encoded by the library of variant nuclei acids to replace tryptophan, a third variant with any codon to replace isoleucine, a fourth variant with any codon to replace lysine, a fifth variant with any codon to replace arginine, a sixth variant with any codon to replace glutamic acid, and a seventh variant with any codon to replace glutamine. When all or less than all codon variants are encoded by the variant nucleic acid library, a resulting corresponding population of amino acid sequence variants is generated following protein expression (i.e., standard cellular events of DNA transcription followed by translation and processing events).

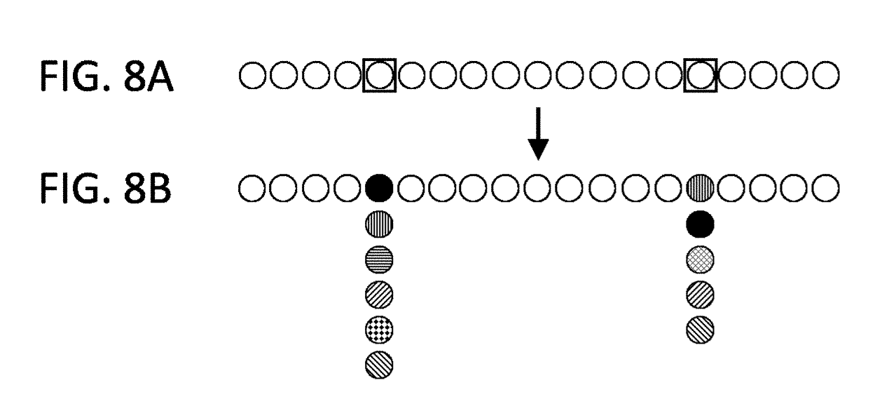

In some arrangements, a library is generated with multiple sites of single position variants. As depicted in FIG. 8A, a wild-type template is provided. FIG. 8B depicts the resultant amino acid sequence with two sites of single position codon variants, wherein each codon variant encoding for a different amino acid is indicated by differently patterned circles.

Provided herein are methods to generate a library having a stretch of multiple site, single position variants. Each stretch of nucleic acid may have 1, 2, 3, 4, 5, or more variants. Each stretch of nucleic acid may have at least 1 variant. Each stretch of nucleic acid may have at least 2 variants. Each stretch of nucleic acid may have at least 3 variants. For example, a stretch of 5 nucleic acids may have 1 variant. A stretch of 5 nucleic acids may have 2 variants. A stretch of 5 nucleic acids may have 3 variants. A stretch of 5 nucleic acids may have 4 variants. For example, a stretch of 4 nucleic acids may have 1 variant. A stretch of 4 nucleic acids may have 2 variants. A stretch of 4 nucleic acids may have 3 variants. A stretch of 4 nucleic acids may have 4 variants.

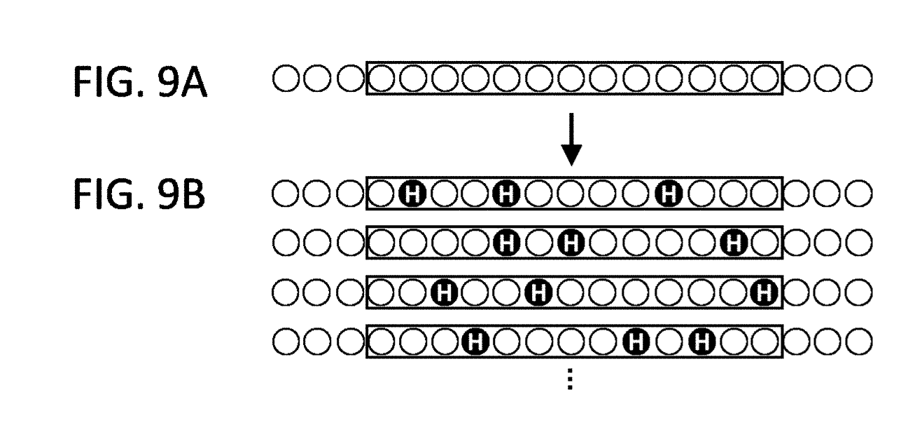

In some instances, single position variants may all encode for the same amino acid, e.g. a histidine. As depicted in FIG. 9A, a reference amino acid sequence is provided. In this arrangement, a stretch of a nucleic acid encodes for multiple sites of single position variants and, when expressed, results in an amino acid sequence having all single position variants encoding for a histidine, FIG. 9B. In some embodiments, a variant library synthesized by methods described herein does not encode for more than 4 histidine residues in a resultant amino acid sequence.

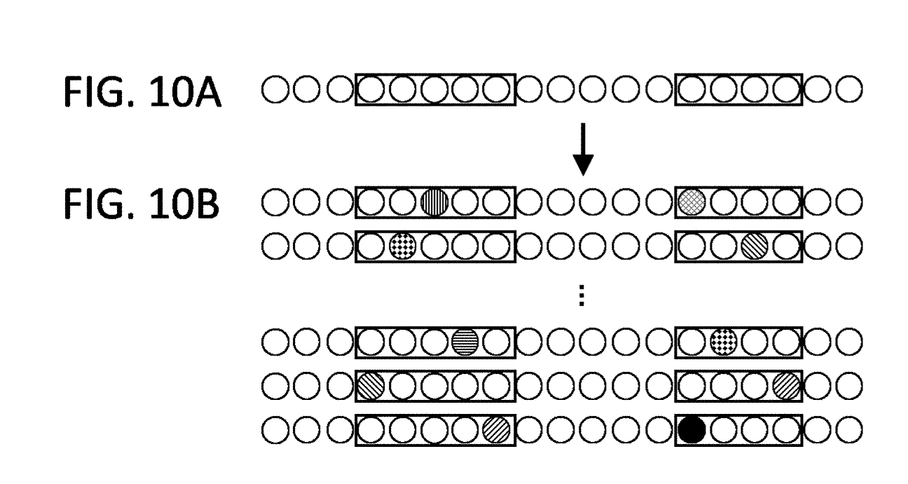

In some instances, a variant library of nucleic acids generated by methods described herein provides for expression of amino acid sequences that have separate stretches of variation. A template amino acid sequence is depicted in FIG. 10A. A stretch of nucleic acids may have only 1 variant codon in two stretches and, when expressed, result in an amino acid sequence depicted in FIG. 10B. Variants are depicted in FIG. 10B by the differently patterned circles to indicate variation in amino acids at different positions in a single stretch.

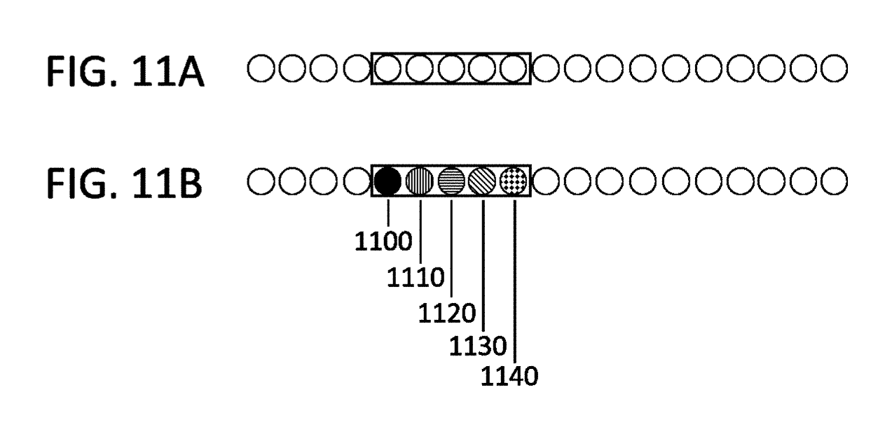

Provided herein are methods and devices to synthesize nucleic acid libraries with 1, 2, 3, or more codon variants, wherein the variant for each site is selectively controlled. The ratio of two amino acids for a single site variant may be about 1:100, 1:50, 1:10, 1:5, 1:3, 1:2, 1:1. The ratio of three amino acids for a single site variant may be about 1:1:100, 1:1:50, 1:1:20, 1:1:10, 1:1:5, 1:1:3, 1:1:2, 1:1:1, 1:10:10, 1:5:5, 1:3:3, or 1:2:2. FIG. 11A depicts a wild-type reference amino acid sequence encoded by a wild-type nucleic acid sequence. FIG. 11B depicts a library of amino acid variants, wherein each variant comprising a stretch of sequence (indicated by the patterned circles), wherein each position may have a certain ratio of amino acids in the resultant variant protein library. The resultant variant protein library is encoded by a variant nucleic acid library generated by methods described herein. In this illustration, 5 positions are varied: the first position 1100 has a 50/50 K/R ratio; the second position 1110 has a 50/25/25 V/L/S ratio, the third position 1120 has a 50/25/25 Y/R/D ratio, the fourth position 1130 has an equal ratio for all 20 amino acids, and the fifth position 1140 has a 75/25 ratio for G/P. The ratios described herein are exemplary only.

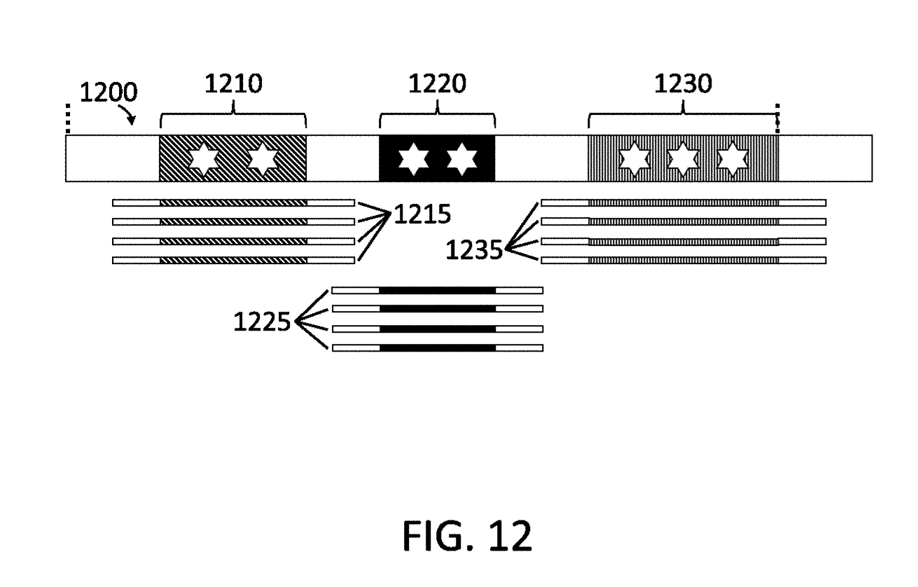

In some instances, a synthesized variant library is generated which encodes for a nucleic acid sequence that is ultimately translated into an amino acid sequence of a protein. Exemplary amino acid sequences includes those encoding for small peptides as well as at least a portion of large peptides, e.g., antibody sequence. In some instances, the nucleic acids synthesized each encode for a variant codon in a portion of an antibody sequence. Exemplary antibody sequences for which the portion of variant synthesized nucleic acid encodes includes the antigen-binding or variable region thereof, or a fragment thereof. Examples of antibody fragments for which the nucleic acids described herein encode a portion of include, without limitation, Fab, Fab′, F(ab′)2 and Fv fragments, diabodies, linear antibodies, single-chain antibody molecules, and multispecific antibodies formed from antibody fragments. Examples antibody regions for which the nucleic acids described herein encode a portion of include, without limitation, Fc region, Fab region, variable region of the Fab region, constant region of the Fab region, variable domain of the heavy chain or light chain (V_{H} or V_{L}), or specific complementarity-determining regions (CDRs) of V_{H} or V_{L}. Variant libraries generated by methods disclosed herein can result in variation of one or more of the antibody regions described herein. In one exemplary process, a variant library is generated for nucleic acids encoding for a several CDRs. See FIG. 12. A template nucleic acid encoding for an antibody having CDR1 1210, CDR2 1220, and CDR3 1230 regions, is modified by methods described herein, where each CDR region comprises multiple sites for variation. Variations for each of 3 CDRs in a single variable domain of a heavy chain or light chain 1215, 1225, and 1235 are generated. Each site, indicated by a star, may comprise a single position, a stretch of multiple, consecutive positions, or both, that are interchangeable with any codon sequence different from the template nucleic acid sequence. Diversity of variant libraries may dramatically increase using methods provided herein, with up to ˜10¹⁰ diversity, or more.

In some instances, variant libraries comprise single or multiple variants of a variable domain of a heavy chain or a light chain (V_{H} or V_{L}). In some instances, variant libraries comprise single or multiple variants in a V_{H} region. Exemplary V_{H} regions include, but are not limited to, IGHV1, IGHV2, IGHV3, IGHV4, IGHV5, IGHV6, and IGHV7. In some instances, variant libraries comprise single or multiple variants in a V_{L} region. Exemplary V_{L} regions include, but are not limited to, IGKV1, IGKV2, IGKV3, IGKV4, IGKV5, IGLV1, IGLV2, and IGLV3.

Variation in Expression Cassettes

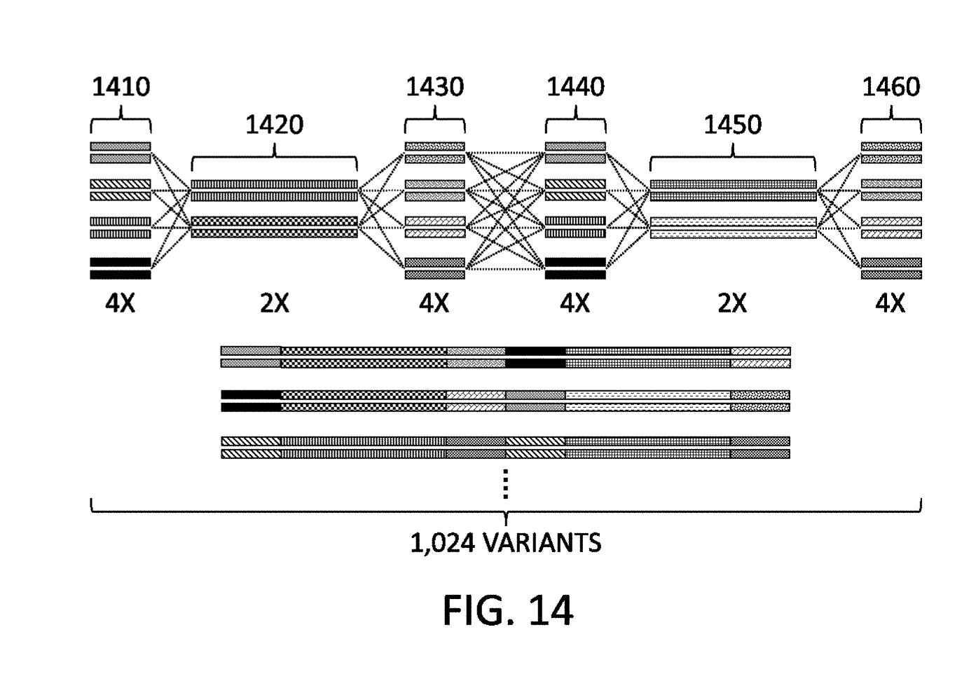

In some instances, a synthesized variant library is generated which encodes for a portion of an expression construct. Exemplary portions of an expression construct include the promoter, open reading frame, and termination region. In some instances, the expression construct encodes for one, two, three or more expression cassettes. A nucleic acid library may be generated, encoding for codon variation at a single site or multiple sites separate regions that make up potions of an expression construct cassette, as depicted in FIG. 14. To generate a two construct expressing cassette, variant nucleic acids were synthesized encoding at least a portion of a variant sequence of a first promoter 1410, first open reading frame 1420, first terminator 1430, second promoter 1440, second open reading frame 1450, or second terminator sequence 1460. After rounds of amplification, as described in previous examples, a library of 1,024 expression constructs was generated. FIG. 14 provides but one example arrangement. In some instances, additional regulator sequences, such as untranslated regulatory region (UTR) or an enhancer region, is are also included in an expression cassette referred to herein. An expression cassette may comprise 1, 2, 3, 4, 5, 6, 7, 8, 9, 10 or more components for which variant sequences are generated by methods described herein. In some instances, the expression construct comprises more than one gene in a multicistronic vector. In one example, the synthesized DNA nucleic acids are inserted into viral vectors (e.g., a lentivirus) and then packaged for transduction into cells, or non-viral vectors for transfer into cells, followed by screening and analysis.

Expression vectors for inserting nucleic acids disclosed herein comprise eukaryotic (e.g., bacterial and fungal) and prokaryotic (e.g., mammalian, plant and insect expression vectors). Exemplary expression vectors include, without limitation, mammalian expression vectors: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG(R)-6His (“6His” disclosed as SEQ ID NO: 32), pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. Exemplary cells include without limitation, prokaryotic and eukaryotic cells. Exemplary eukaryotic cells include, without limitation, animal, plant, and fungal cells. Exemplary animal cells include, without limitation, insect, fish and mammalian cells. Exemplary mammalian cells include mouse, human, and primate cells. Nucleic acids synthesized by methods described herein may be transferred into cells done by various methods known in the art, including, without limitation, transfection, transduction, and electroporation. Exemplary cellular functions tested include, without limitation, changes in cellular proliferation, migration/adhesion, metabolic, and cell-signaling activity.

Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can is optimized using methods described herein. For example, to improve a specified function of an antibody, a variant nucleic acid library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (V_{H} or V_{L}), and specific complementarity-determining regions (CDRs) of V_{H} or V_{L}.

Alternatively, the molecule to optimize is a receptor binding epitope for use as an activating agent or competitive inhibitor. Subsequent to synthesis of a variant library of nucleic acids, the variant library of nucleic acids may be inserted into a vector sequence and then expressed in cells. The receptor antigen may be expressed in cells (e.g., insect, mammalian or bacterial) and then purified, or it may be expressed in cells (e.g., mammalian) to examine a functional consequence from variation of the sequence. Functional consequences include, without limitation, a change in the proteins expression, binding affinity and stability. Cellular functional consequence include, without limitation, a change in reproduction, growth, adhesion, death, migration, energy production, oxygen utilization, metabolic activity, cell signaling, aging, response to free radical damage, or any combination thereof. In some embodiments, the type of protein selected for optimization is an enzyme, transporter proteins, G-protein coupled receptors, voltage-gated ion channels, transcription factors, polymerases, adaptor proteins (proteins without enzymatic activity the serve to bring two other proteins together), and cytoskeletal proteins. Exemplary types of enzymes include, without limitation, signaling enzymes (such as protein kinases, protein phosphatases, phosphodiesterases, histone deacteylases, and GTPases).

Provided herein are variant nucleic acid libraries comprising variants for molecules involved in an entire pathway or an entire genome. Exemplary pathways include, without limitation a metabolic, cell death, cell cycle progression, immune cell activation, inflammatory response, angiogenesis, lymphogenesis, hypoxia and oxidative stress response, or cell adhesion/migration pathway. Exemplary proteins in a cell death pathway include, without limitation, Fas, Cadd, Caspase 3, Caspase 6, Caspase 8, Caspase 9, Caspase 10, IAP, TNFR1, TNF, TNFR2, NF-kB, TRAFs, ASK, BAD, and Akt. Exemplary proteins in a cell cycle pathway include, without limitation, NFkB, E2F, Rb, p53, p21, cyclin A, cyclin B, cyclin D, cyclin E, and cdc 25. Exemplary proteins in a cell migration pathway include, without limitation, Ras, Raf, PLC, cofilin, MEK, ERK, MLP, LIMK, ROCK, RhoA, Src, Rac, Myosin II, ARP2/3, MAPK, PIP2, integrins, talin, kindlin, migfilin and filamin.

Nucleic acid libraries synthesized by methods described herein may be expressed in various cell types. Exemplary cell types include prokaryotes (e.g., bacteria and fungi) and eukaryotes (e.g., plants and animals). Exemplary animals include, without limitation, mice, rabbits, primates, fish, and insects. Exemplary plants include, without limitation, a monocot and dicot. Exemplary plants also include, without limitation, microalgae, kelp, cyanobacteria, and green, brown and red algae, wheat, tobacco, and corn, rice, cotton, vegetables, and fruit.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

Nucleic acid libraries synthesized by methods described herein may be expressed in various cell types assess a change in cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

To identify a variant molecule associated with prevention, reduction or treatment of a disease state, a variant nucleic acid library described herein is expressed in a cell associated with a disease state, or one in which a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided herein are less than 1/500 or less compared to the predetermined sequences.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large nucleic acid and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic nucleic acid and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

Provided herein are methods and devices for manufacture and growth of polynucleotides about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the polynucleotide formed is about 5, 10, 20, 30, 40, 50, 60, 70, 80, 90, 100, 125, 150, 175, 200, or 225 bases in length. A polynucleotide may be at least 5, 10, 20, 30, 40, 50, 60, 70, 80, 90, or 100 bases in length. A polynucleotide may be from 10 to 225 bases in length, from 12 to 100 bases in length, from 20 to 150 bases in length, from 20 to 130 bases in length, or from 30 to 100 bases in length.

In some instances, polynucleotides are synthesized on distinct loci of a substrate, wherein each locus supports the synthesis of a population of polynucleotides. In some instances, each locus supports the synthesis of a population of polynucleotides having a different sequence than a population of polynucleotides grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500, 1000 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct polynucleotides synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 um to about 500 um, from about 10 um to about 200 um, or from about 10 um to about 100 um. In some instances, the distance from two centers of adjacent loci is greater than about 10 um, 20 um, 30 um, 40 um, 50 um, 60 um, 70 um, 80 um, 90 um or 100 um. In some instances, the distance from the centers of two adjacent loci is less than about 200 um, 150 um, 100 um, 80 um, 70 um, 60 um, 50 um, 40 um, 30 um, 20 um or 10 um. In some instances, each locus has a width of about 0.5 um, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um or 100 um. In some instances, each locus has a width of about 0.5 um to 100 um, about 0.5 um to 50 um, about 10 um to 75 um, or about 0.5 um to 50 um.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 um, 100 um, 200 um, 500 um, 1000 um, or 2000 um or 5000 um. In some instances, the distance from the centers of two adjacent clusters is from about 50 um to about 100 um, from about 50 um to about 200 um, from about 50 um to about 300 um, from about 50 um to about 500 um, and from about 100 um to about 2000 um. In some instances, the distance from the centers of two adjacent clusters is from about 0.05 mm to about 50 mm, from about 0.05 mm to about 10 mm, from about 0.05 mm to about 5 mm, from about 0.05 mm to about 4 mm, from about 0.05 mm to about 3 mm, from about 0.05 mm to about 2 mm, from about 0.1 mm to about 10 mm, from about 0.2 mm to about 10 mm, from about 0.3 mm to about 10 mm, from about 0.4 mm to about 10 mm, from about 0.5 mm to about 10 mm, from about 0.5 mm to about 5 mm, or from about 0.5 mm to about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm to 1000 mm, from about 25 mm to about 800 mm, from about 25 mm to about 600 mm, from about 25 mm to about 500 mm, from about 25 mm to about 400 mm, from about 25 mm to about 300 mm, or from about 25 mm to about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm to about 2000 mm, from about 50 mm to about 1000 mm, from about 100 mm to about 1000 mm, from about 200 mm to about 1000 mm, or from about 250 mm to about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

Surface Materials

Provided herein is a device comprising a surface, wherein the surface is modified to support polynucleotide synthesis at predetermined locations and with a resulting low error rate, a low dropout rate, a high yield, and a high oligo representation. In some embodiments, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provided as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a solid support described herein has a surface with a flexibility of at least nylon.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a material deposition device. In some instances, reagents and/or fluids collect in a larger well in fluid communication with one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, a polynucleotide library may span the length of about 1%, 2%, 3%, 4%, 5%, 10%, 15%, 20%, 30%, 40%, 50%, 60%, 70%, 80%, 90%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 4%, 5%, 10%, 15%, 20%, 30%, 40%, 50%, 60%, 70%, 80%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm to about 5 mm, from about 0.05 mm to about 4 mm, from about 0.05 mm to about 3 mm, from about 0.05 mm to about 2 mm, from about 0.05 mm to about 1 mm, from about 0.05 mm to about 0.5 mm, from about 0.05 mm to about 0.1 mm, from about 0.1 mm to about 10 mm, from about 0.2 mm to about 10 mm, from about 0.3 mm to about 10 mm, from about 0.4 mm to about 10 mm, from about 0.5 mm to about 10 mm, from about 0.5 mm to about 5 mm, or from about 0.5 mm to about 2 mm. In some instances, the width of a well comprising a cluster is from about 0.05 mm to about 50 mm, from about 0.05 mm to about 10 mm, from about 0.05 mm to about 5 mm, from about 0.05 mm to about 4 mm, from about 0.05 mm to about 3 mm, from about 0.05 mm to about 2 mm, from about 0.05 mm to about 1 mm, from about 0.05 mm to about 0.5 mm, from about 0.05 mm to about 0.1 mm, from about 0.1 mm to about 10 mm, from about 0.2 mm to about 10 mm, from about 0.3 mm to about 10 mm, from about 0.4 mm to about 10 mm, from about 0.5 mm to about 10 mm, from about 0.5 mm to about 5 mm, or from about 0.5 mm to about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 to about 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 um to about 1000 um, from about 50 um to about 1000 um, from about 100 um to about 1000 um, from about 200 um to about 1000 um, from about 300 um to about 1000 um, from about 400 um to about 1000 um, or from about 500 um to about 1000 um. In some instances, the height of a well is less than about 1000 um, less than about 900 um, less than about 800 um, less than about 700 um, or less than about 600 um.

In some instances, a device comprises a plurality of channels corresponding to a plurality of loci within a cluster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a channel is less than 100 um, less than 80 um, less than 60 um, less than 40 um or less than 20 um.

In some instances, the diameter of a channel, locus (e.g., in a substantially planar substrate) or both channel and locus (e.g., in a three-dimensional device wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 90 um, 80 um, 70 um, 60 um, 50 um, 40 um, 30 um, 20 um or 10 um. In some instances, the diameter of a channel, locus, or both channel and locus is less than about 100 um, 90 um, 80 um, 70 um, 60 um, 50 um, 40 um, 30 um, 20 um or 10 um. In some instances, the distance from the center of two adjacent channels, loci, or channels and loci is from about 1 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um.

Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which polynucleotides or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification of layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyetheyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein include, without limitation, N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis (3-trimethoxysilylpropyl) amine.

Exemplary passive agents for inclusion in a coating material described herein include, without limitation, perfluorooctyltrichlorosilane; tridecafluoro-1,1,2,2-tetrahydrooctyl)trichlorosilane; 1H, 1H, 2H, 2H-fluorooctyltriethoxysilane (FOS); trichloro(1H, 1H, 2H, 2H-perfluorooctyl)silane; tert-butyl-[5-fluoro-4-(4,4,5,5-tetramethyl-1,3,2-dioxaborolan-2-yl)indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing polynucleotide chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is deprotected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing polynucleotide is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150, 22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on a substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein a polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with a low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

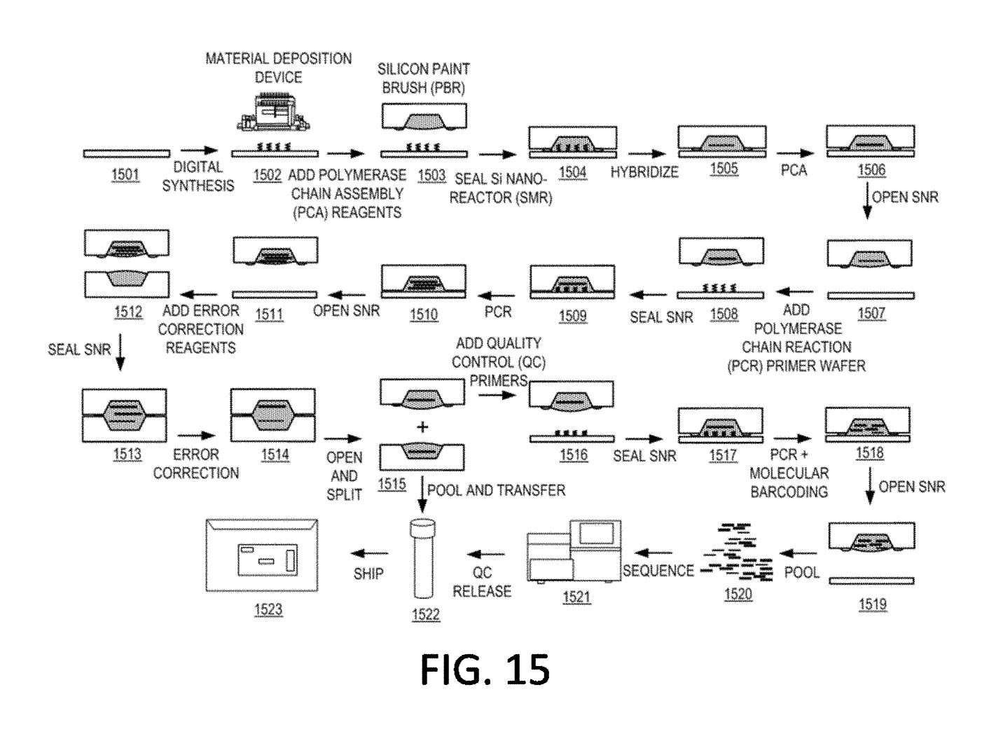

Referring to the Figures, FIG. 15 illustrates an exemplary process workflow for synthesis of nucleic acids (e.g., genes) from shorter polynucleotides. The workflow is divided generally into phases: (1) de novo synthesis of a single stranded polynucleotide acid library, (2) joining polynucleotides to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of polynucleotides is designed for de novo synthesis. Various suitable methods are known for generating high density polynucleotide arrays. In the workflow example, a device surface layer 1501 is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 1502. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 1503. Prior to or after the sealing 1504 of the polynucleotides, a reagent is added to release the polynucleotides from the substrate. In the exemplary workflow, the polynucleotides are released subsequent to sealing of the nanoreactor 1505. Once released, fragments of single stranded polynucleotides hybridize in order to span an entire long range sequence of DNA. Partial hybridization 1505 is possible because each synthesized polynucleotide is designed to have a small portion overlapping with at least one other polynucleotide in the population.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 1506.

After PCA is complete, the nanoreactor is separated from the device 1507 and positioned for interaction with a device having primers for PCR 1508. After sealing, the nanoreactor is subject to PCR 1509 and the larger nucleic acids are amplified. After PCR 1510, the nanochamber is opened 1511, error correction reagents are added 1512, the chamber is sealed 1513 and an error correction reaction occurs to remove mismatched base pairs and/or strands with poor complementarity from the double stranded PCR amplification products 1514. The nanoreactor is opened and separated 1515. Error corrected product is next subject to additional processing steps, such as PCR and molecular bar coding, and then packaged 1522 for shipment 1523.

In some instances, quality control measures are taken. After error correction, quality control steps include for example interaction with a wafer having sequencing primers for amplification of the error corrected product 1516, sealing the wafer to a chamber containing error corrected amplification product 1517, and performing an additional round of amplification 1518. The nanoreactor is opened 1519 and the products are pooled 1520 and sequenced 1521. After an acceptable quality control determination is made, the packaged product 1522 is approved for shipment 1523.

In some instances, a polynucleotide generated by a workflow such as that in FIG. 15 is subject to mutagenesis using overlapping primers disclosed herein. In some instances, a library of primers is generated by in situ preparation on a solid support and utilize single nucleotide extension process to extend multiple oligomers in parallel. A deposition device, such as material deposition device, is designed to release reagents in a step wise fashion such that multiple polynucleotides extend, in parallel, one residue at a time to generate oligomers with a predetermined nucleic acid sequence 1502.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

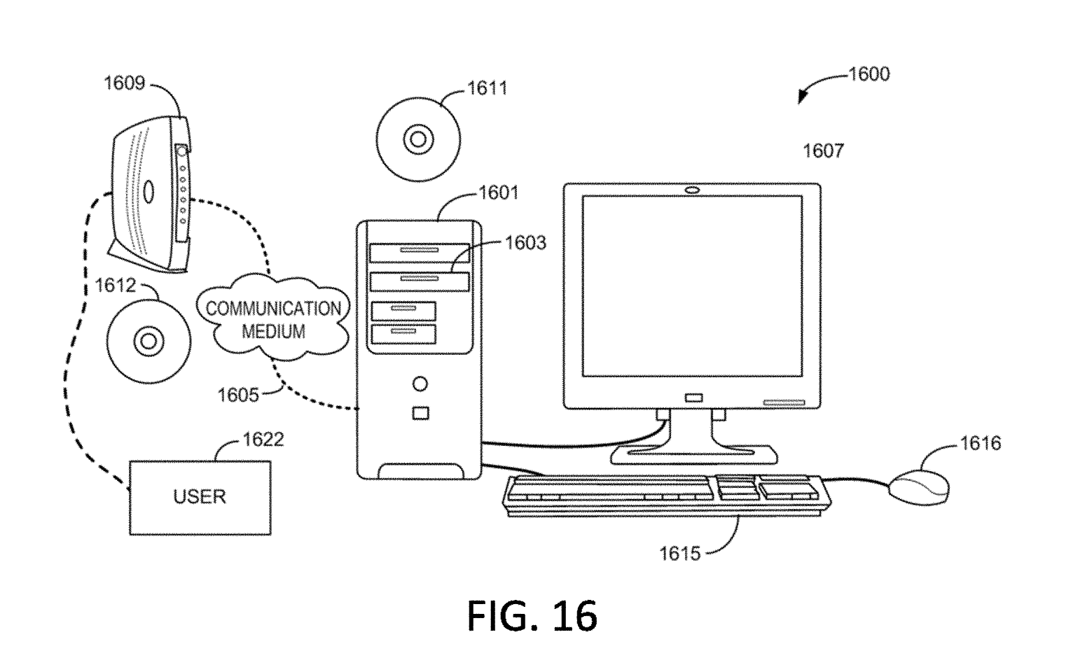

The computer system 1600 illustrated in FIG. 16 may be understood as a logical apparatus that can read instructions from media 1611 and/or a network port 1605, which can optionally be connected to server 1609 having fixed media 1612. The system, such as shown in FIG. 16 can include a CPU 1601, disk drives 1603, optional input devices such as keyboard 1615 and/or mouse 1616 and optional monitor 1607. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1622 as illustrated in FIG. 16.

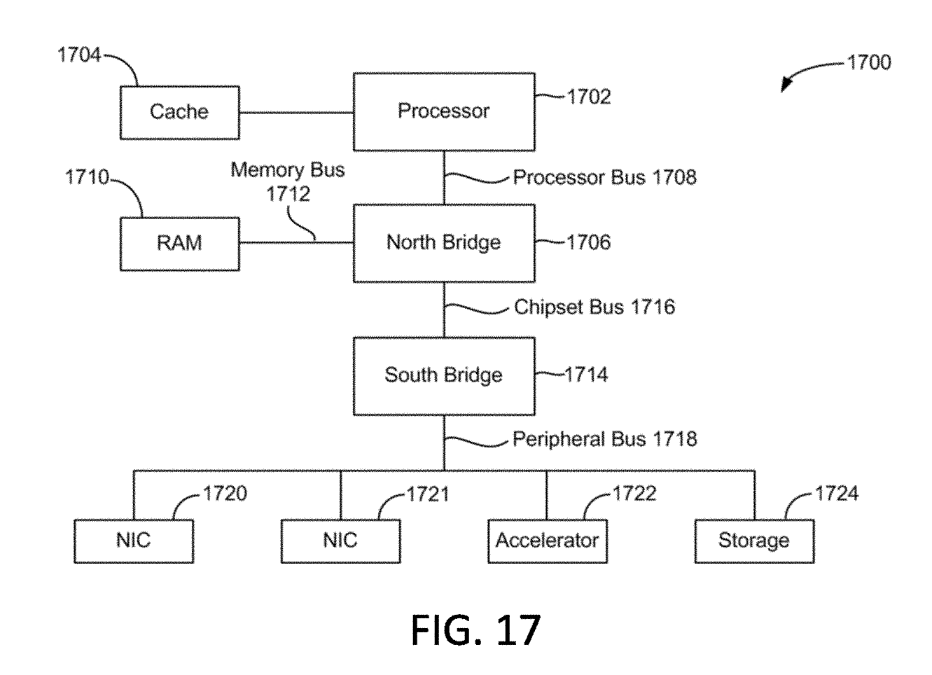

FIG. 17 is a block diagram illustrating a first example architecture of a computer system 1700 that can be used in connection with example instances of the present disclosure. As depicted in FIG. 17, the example computer system can include a processor 1702 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 17, a high speed cache 1704 can be connected to, or incorporated in, the processor 1702 to provide a high speed memory for instructions or data that have been recently, or are frequently, used by processor 1702. The processor 1702 is connected to a north bridge 1706 by a processor bus 1708. The north bridge 1706 is connected to random access memory (RAM) 1710 by a memory bus 1712 and manages access to the RAM 1710 by the processor 1702. The north bridge 1706 is also connected to a south bridge 1714 by a chipset bus 1716. The south bridge 1714 is, in turn, connected to a peripheral bus 1718. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1718. In some alternative architectures, the functionality of the north bridge can be incorporated into the processor instead of using a separate north bridge chip. In some instances, system 1700 can include an accelerator card 1722 attached to the peripheral bus 1718. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1724 and can be loaded into RAM 1710 and/or cache 1704 for use by the processor. The system 1700 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1700 also includes network interface cards (NICs) 1720 and 1721 connected to the peripheral bus for providing network interfaces to external storage, such as Network Attached Storage (NAS) and other computer systems that can be used for distributed parallel processing.

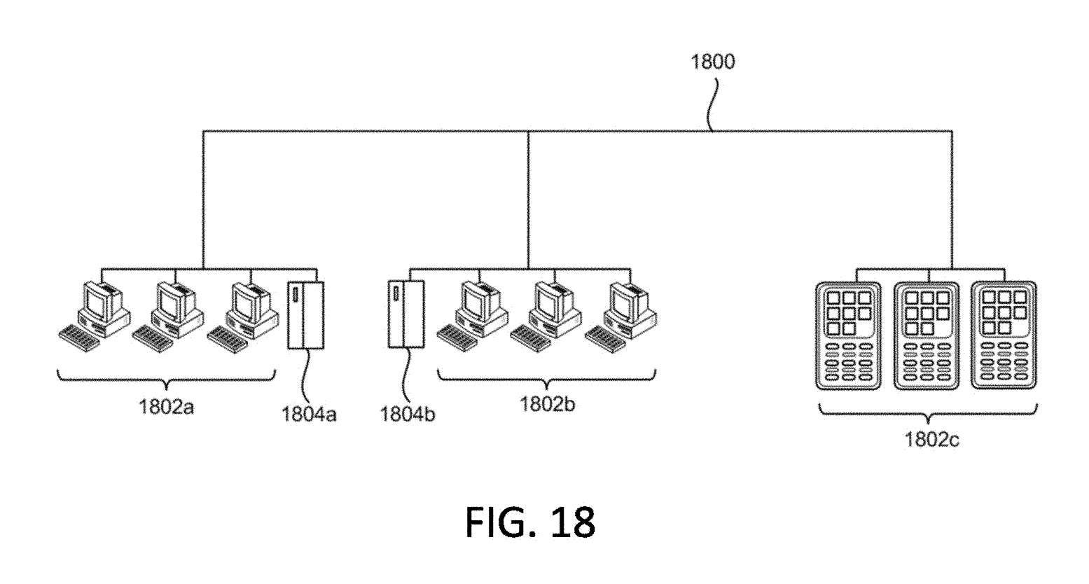

FIG. 18 is a diagram showing a network 1800 with a plurality of computer systems 1802a, and 1802b, a plurality of cell phones and personal data assistants 1802c, and Network Attached Storage (NAS) 1804a, and 1804b. In example instances, systems 1802a, 1802b, and 1802c can manage data storage and optimize data access for data stored in Network Attached Storage (NAS) 1804a and 1804b. A mathematical model can be used for the data and be evaluated using distributed parallel processing across computer systems 1802a, and 1802b, and cell phone and personal data assistant systems 1802c. Computer systems 1802a, and 1802b, and cell phone and personal data assistant systems 1802c can also provide parallel processing for adaptive data restructuring of the data stored in Network Attached Storage (NAS) 1804a and 1804b. FIG. 18 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

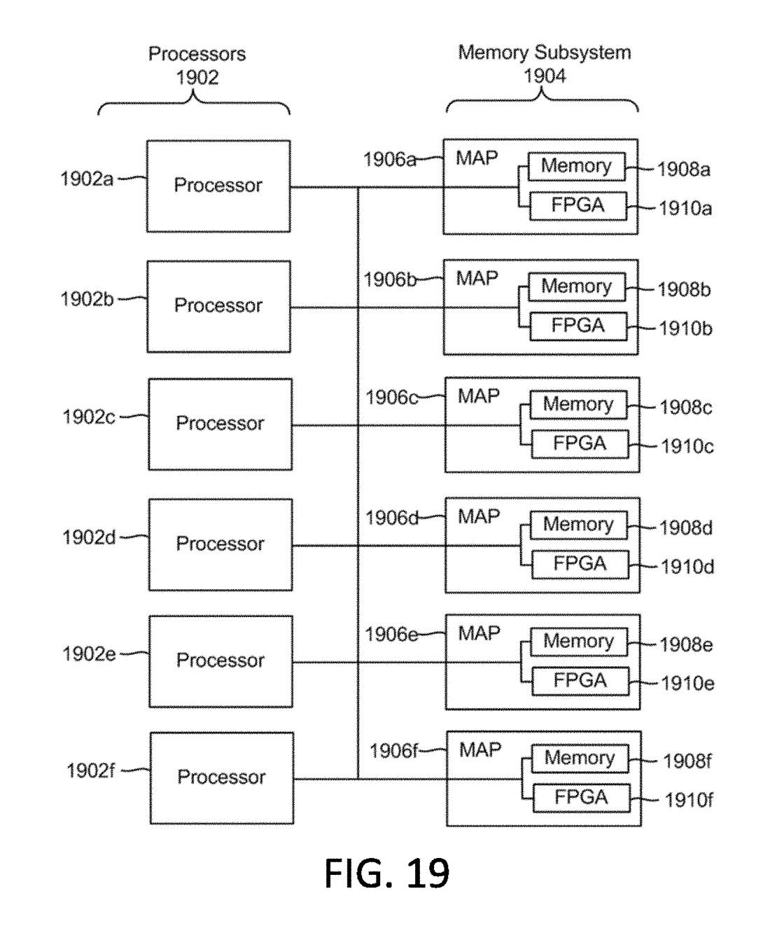

FIG. 19 is a block diagram of a multiprocessor computer system 1900 using a shared virtual address memory space in accordance with an example instance. The system includes a plurality of processors 1902a-f that can access a shared memory subsystem 1904. The system incorporates a plurality of programmable hardware memory algorithm processors (MAPs) 1906a-f in the memory subsystem 1904. Each MAP 1906a-f can comprise a memory 1908a-f and one or more field programmable gate arrays (FPGAs) 1910a-f. The MAP provides a configurable functional unit and particular algorithms or portions of algorithms can be provided to the FPGAs 1910a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1908a-f, allowing it to execute tasks independently of, and asynchronously from the respective microprocessor 1902a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 19, system on chips (SOCs), application specific integrated circuits (ASICs), or other processing and logic elements. For example, the Set Processor and Optimizer can be implemented with hardware acceleration through the use of a hardware accelerator card, such as accelerator card 1722 illustrated in FIG. 17.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200× spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A cleaning process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Synthesis of a 50-Mer Sequence

A two-dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 20. 5′AGACAATCAACCATTTGGGGTGGACAGCCTTGACCTCTAGACTTCGGCAT##TTTTT TTTTT3′ (SEQ ID NO.: 20), where # denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) according to the protocol in Table 4 and an ABI synthesizer.

TABLE 4
Synthesis Protocol

| General DNA Synthesis | Table 4 | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-Mer Sequence

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCAT GCTAGCCATACCATGATGATGATGATGATGAGAACCCCGCAT##TTTTTTTTTT3′, where # denotes Thymidine-succinyl hexamide CED phosphoramidite (CLP-2244 from ChemGenes); SEQ ID NO.: 21) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO.: 22) and a reverse (5′CGGGATCCTTATCGTCATCG3′; SEQ ID NO.: 23) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermalcycling program:

98° C., 30 sec

98° C., 10 sec; 63° C., 10 sec; 72° C., 10 sec; repeat 12 cycles

72° C., 2 min

The PCR products were also run on a BioAnalyzer (data not shown), demonstrating sharp peaks at the 100-mer position. Next, the PCR amplified samples were cloned, and Sanger sequenced. Table 5 summarizes the results from the Sanger sequencing for samples taken from spots 1-5 from chip 1 and for samples taken from spots 6-10 from chip 2.

TABLE 5
Sequencing Results

| | | |
|---|---|---|
| Spot | Error rate | Cycle efficiency |

| | | | |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors. Finally, Table 6 summarizes error characteristics for the sequences obtained from the polynucleotides samples from spots 1-10.

TABLE 6
Error Characteristics

| | Sample ID/Spot no. | | | | |
|---|---|---|---|---|---|
| | OSA_0046/1 | OSA_0047/2 | OSA_0048/3 | OSA_0049/4 | OSA_0050/5 |
| Total | 32 | 32 | 32 | 32 | 32 |
| Sequences | | | | | |
| Sequencing | 25 of | 27 of | 26 of | 21 of | 25 of |
| Quality | 28 | 27 | 30 | 23 | 26 |
| Oligo | 23 of | 25 of | 22 of | 18 of | 24 of |
| Quality | 25 | 27 | 26 | 21 | 25 |
| ROI Match | 2500 | 2698 | 2561 | 2122 | 2499 |
| Count | | | | | |
| ROI | 2 | 2 | 1 | 3 | 1 |
| Mutation | | | | | |
| ROI Multi | 0 | 0 | 0 | 0 | 0 |
| Base | | | | | |
| Deletion | | | | | |
| ROI Small | 1 | 0 | 0 | 0 | 0 |
| Insertion | | | | | |
| ROI | 0 | 0 | 0 | 0 | 0 |
| Single | | | | | |
| Base | | | | | |
| Deletion | | | | | |
| Large | 0 | 0 | 1 | 0 | 0 |
| Deletion | | | | | |
| Count | | | | | |
| Mutation: | 2 | 2 | 1 | 2 | 1 |
| G > A | | | | | |
| Mutation: | 0 | 0 | 0 | 1 | 0 |
| T > C | | | | | |
| ROI Error | 3 | 2 | 2 | 3 | 1 |
| Count | | | | | |
| ROI Error | Err: ~1 | Err: ~1 | Err: ~1 | Err: ~1 | Err: ~1 |
| Rate | in 834 | in 1350 | in 1282 | in 708 | in 2500 |
| ROI | MP Err: | MP Err: | MP Err: | MP Err: | MP Err: |
| Minus | ~1 in | ~1 in | ~1 in | ~1 in | ~1 in |
| Primer | 763 | 824 | 780 | 429 | 1525 |
| Error Rate | | | | | |
| | Sample ID/Spot no. | | | | |
| | OSA_0051/6 | OSA_0052/7 | OSA_0053/8 | OSA_0054/9 | OSA_0055/10 |
| Total | 32 | 32 | 32 | 32 | 32 |
| Sequences | | | | | |
| Sequencing | 29 of | 27 of | 29 of | 28 of | 25 of |
| Quality | 30 | 31 | 31 | 29 | 28 |
| Oligo | 25 of | 22 of | 28 of | 26 of | 20 of |
| Quality | 29 | 27 | 29 | 28 | 25 |
| ROI Match | 2666 | 2625 | 2899 | 2798 | 2348 |
| Count | | | | | |
| ROI | 0 | 2 | 1 | 2 | 1 |
| Mutation | | | | | |
| ROI Multi | 0 | 0 | 0 | 0 | 0 |
| Base | | | | | |
| Deletion | | | | | |
| ROI Small | 0 | 0 | 0 | 0 | 0 |
| Insertion | | | | | |
| ROI | 0 | 0 | 0 | 0 | 0 |
| Single | | | | | |
| Base | | | | | |
| Deletion | | | | | |
| Large | 1 | 1 | 0 | 0 | 0 |
| Deletion | | | | | |
| Count | | | | | |
| Mutation: | 0 | 2 | 1 | 2 | 1 |
| G > A | | | | | |
| Mutation: | 0 | 0 | 0 | 0 | 0 |
| T > C | | | | | |
| ROI Error | 1 | 3 | 1 | 2 | 1 |
| Count | | | | | |
| ROI Error | Err: ~1 | Err: ~1 | Err: ~1 | Err: ~1 | Err: ~1 |
| Rate | in 2667 | in 876 | in 2900 | in 1400 | in 2349 |
| ROI | MP Err: | MP Err: | MP Err: | MP Err: | MP Err: |
| Minus | ~1 in | ~1 in | ~1 in | ~1 in | ~1 in |
| Primer | 1615 | 531 | 1769 | 854 | 1451 |
| Error Rate | | | | | |

### Example 4: Generation of a Nucleic Acid Library by Single-Site, Single Position Mutagenesis

Polynucleotide primers were de novo synthesized for use in a series of PCR reactions to generate a library of nucleic acid variants of a template nucleic acid, see FIGS. 4A-4D. Four types of primers were generated in FIG. 4A: an outer 5′ primer 415, an outer 3′ primer 430, an inner 5′ primer 425, and an inner 3′ primer 420. The inner 5′ primer/first polynucleotide 420 and an inner 3′ primer/second polynucleotide 425 were generated using a polynucleotide synthesis method as generally outlined in Table 4. The inner 5′ primer/first polynucleotide 420 represents a set of up to 19 primers of predetermined sequence, where each primer in the set differs from another at a single codon, in a single site of the sequence.

Polynucleotide synthesis was performed on a device having at least two clusters, each cluster having 121 individually addressable loci.

The inner 5′ primer 425 and the inner 3′ primer 420 were synthesized in separate clusters. The inner 5′ primer 425 was replicated 121 times, extending on 121 loci within a single cluster. For inner 3′ primer 420, each of the 19 primers of variant sequences were each extended on 6 different loci, resulting in the extension of 114 polynucleotides on 114 different loci.

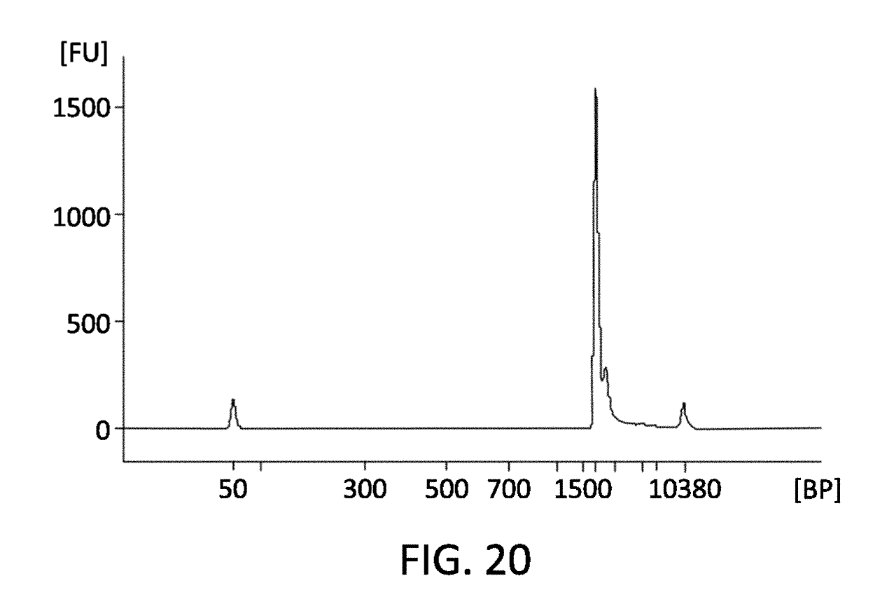

Synthesized polynucleotide were cleaved from the surface of the device and transferred to a plastic vial. A first PCR reaction was performed, using fragments of the long nucleic acid sequence 435, 440 to amplify the template nucleic acid, as illustrated in FIG. 4B. A second PCR reaction was performed using primer combination and the products of the first PCR reaction as a template, as illustrated in FIGS. 4C-4D. Analysis of the second PCR products was conducted on a BioAnalyzer, as shown in the trace of FIG. 20.

### Example 5: Generation of a Nucleic Acid Library Comprising 96 Different Sets of Single Position Variants

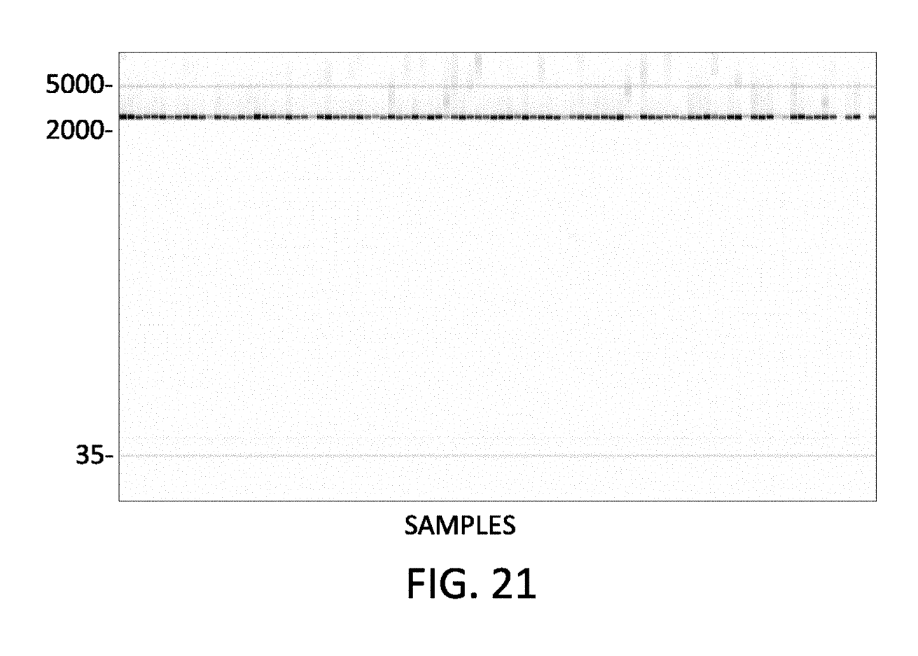

Four sets of primers, as generally shown in FIG. 4A and addressed in Example 2, were generated using de novo polynucleotide synthesis. For the inner 5′ primer 420, 96 different sets of primers were generated, each set of primers targeting a different single codon positioned within a single site of the template nucleic acid. For each set of primers, 19 different variants were generated, each variant comprising a codon encoding for a different amino acid at the single site. Two rounds of PCR were performed using the generated primers, as generally shown in FIGS. 4A-4D and described in Example 2. The 96 sets of amplification products were visualized in an electropherogram (FIG. 21), which was used to calculate a 100% amplification success rate.

### Example 6: Generation of a Nucleic Acid Library Comprising 500 Different Sets of Single Position Variants

Four sets of primers, as generally shown in FIG. 4A and addressed in Example 2, were generated using de novo polynucleotide synthesis. For the inner 5′ primer 420, 500 different sets of primers were generated, each set of primers targeting a different single codon positioned within a single site of the template nucleic acid. For each set of primers, 19 different variants were generated, each variant comprising a codon encoding for a different amino acid at the single site. Two rounds of PCR were performed using the generated primers, as generally shown in FIG. 4A and described in Example 2. Electropherograms display each of the 500 sets of PCR products having a population of nucleic acids with 19 variants at a different single site (data not shown). A comprehensive sequencing analysis of the library showed a greater than 99% success rate across preselected codon mutations (sequence trace and analysis data not shown).

### Example 7: Single-Site Mutagenesis Primers for 1 Position

An example of codon variation design is provided in Table 7 for Yellow Fluorescent Protein. In this case, a single codon from a 50-mer of the sequence is varied 19 times. Variant nucleic acid sequence is indicated by bold letters. The wild type primer sequence is:

(SEQ ID NO.: 1)
ATGGTGAGCAAGGGCGAGGAGCTGTTCACCGGGGTGGTGCCCAT.

In this case, the wild type codon encodes for valine, indicated by underline in SEQ ID NO.: 1. Therefore the 19 variants below excludes a codon encoding for valine. In an alternative example, if all triplets are to be considered, then all 60 variants would be generated, including an alternative sequence for the wild type codon.

TABLE 7
Variant Sequences

| SEQ | | |
|---|---|---|
| ID | | Variant |
| NO. | Variant sequence | codon |
| 2 | atgTTTAGCAAGGGCGAGGAGCTGTTCACCGGGGTGGTGCCCAT | F |
| 3 | atgTTAAGCAAGGGCGAGGAGCTGTTCACCGGGGTGGTGCCCAT | L |
| 4 | atgATTAGCAAGGGCGAGGAGCTGTTCACCGGGGTGGTGCCCAT | I |
| 5 | atgTCTAGCAAGGGCGAGGAGCTGTTCACCGGGGTGGTGCCCAT | S |
| 6 | atgCCTAGCAAGGGCGAGGAGCTGTTCACCGGGGTGGTGCCCAT | P |
| 7 | atgACTAGCAAGGGCGAGGAGCTGTTCACCGGGGTGGTGCCCAT | T |
| 8 | atgGCTAGCAAGGGCGAGGAGCTGTTCACCGGGGTGGTGCCCAT | A |
| 9 | atgTATAGCAAGGGCGAGGAGCTGTTCACCGGGGTGGTGCCCAT | Y |
| 10 | atgCATAGCAAGGGCGAGGAGCTGTTCACCGGGGTGGTGCCCAT | H |
| 11 | atgCAAAGCAAGGGCGAGGAGCTGTTCACCGGGGTGGTGCCCAT | Q |
| 12 | atgAATAGCAAGGGCGAGGAGCTGTTCACCGGGGTGGTGCCCAT | N |
| 13 | atgAAAAGCAAGGGCGAGGAGCTGTTCACCGGGGTGGTGCCCAT | K |
| 14 | atgGATAGCAAGGGCGAGGAGCTGTTCACCGGGGTGGTGCCCAT | D |
| 15 | atgGAAAGCAAGGGCGAGGAGCTGTTCACCGGGGTGGTGCCCAT | E |
| 16 | atgTGTAGCAAGGGCGAGGAGCTGTTCACCGGGGTGGTGCCCAT | C |
| 17 | atgTGGAGCAAGGGCGAGGAGCTGTTCACCGGGGTGGTGCCCAT | W |
| 18 | atgCGTAGCAAGGGCGAGGAGCTGTTCACCGGGGTGGTGCCCAT | R |
| 19 | atgGGTAGCAAGGGCGAGGAGCTGTTCACCGGGGTGGTGCCCAT | G |

### Example 8: Single Site, Dual Position Nucleic Acid Variants

De novo polynucleotide synthesis was performed under conditions similar to those described in Example 2. A single cluster on a device was generated which contained synthesized predetermined variants of a nucleic acid for 2 consecutive codon positions at a single site, each position being a codon encoding for an amino acid. In this arrangement, 19 variants/per position were generated for 2 positions with 3 replicates of each nucleic acid, resulting in 114 nucleic acids synthesized.

### Example 9: Multiple Site, Dual Position Nucleic Acid Variants

De novo polynucleotide synthesis was performed under conditions similar to those described in Example 2. A single cluster on a device was generated which contained synthesized predetermined variants of a nucleic acid for 2 non-consecutive codon positions, each position being a codon encoding for an amino acid. In this arrangement, 19 variants/per position were generated for 2 positions.

### Example 10: Single Stretch, Triple Position Nucleic Acid Variants

De novo polynucleotide synthesis was performed under conditions similar to those described in Example 2. A single cluster on a device was generated which contained synthesized predetermined variants of a reference nucleic acid for 3 consecutive codon positions. In the 3 consecutive codon position arrangement, 19 variants/per position were generated for 3 positions with 2 replicates of each nucleic acid, and resulted in 114 nucleic acids synthesized.

### Example 11: Multiple Site, Triple Position Nucleic Acid Variants

De novo polynucleotide synthesis was performed under conditions similar to those described in Example 2. A single cluster on a device was generated which contains synthesized predetermined variants of a reference nucleic acid for at least 3 non-consecutive codon positions. Within a predetermined region, the location of codons encoding for 3 histidine residues were varied.

### Example 12: Multiple Site, Multiple Position Nucleic Acid Variants

De novo polynucleotide synthesis was performed under conditions similar to those described in Example 2. A single cluster on a device was generated which contained synthesized predetermined variants of a reference nucleic acid for 1 or more codon positions in 1 or more stretches. Five positions were varied in the library. The first position encoded codons for a resultant 50/50 K/R ratio in the expressed protein; the second position encoded codons for a resultant 50/25/25 V/L/S ratio in the expressed protein, the third position encoded codons for a resultant a 50/25/25 Y/R/D ratio in the expressed protein, the fourth position encoded codons for a resultant an equal ratio for all amino acids in the expressed protein, and the fifth position encoded codons for a resultant a 75/25 G/P ratio in the expressed protein.

### Example 13: Generation of Nucleic Acid Libraries by Sampling

To generate a population of nucleic acids with a preselected distribution, computational techniques were used. An example preselected distribution is provided in Table 8 below in which the numbers represent the desired percentage of each amino acid at each position. The cumulative distribution value was first calculated resulting in values from 0.0 to 1.0 as seen in Table 9. In a program such as Excel, a uniform random number generator was used to create values between 0 and 1 for each of ten amino acid positions for 500 nucleic acids used as a sampling population. For example, for position 1, a uniform random value of “0.95” would fall into the “S” bucket and therefore denote the amino acid “S.” This technique is referred to as a “roulette-wheel” selection. Ten random numbers were generated from the 10 discrete distributions for each designed oligonucleotide; this process was repeated 500 times to generate the sample population of 500 nucleic acids. To validate the generated sample population, the sum across the population of the frequency with which each amino acid appears at that position was then determined and expressed as a percentage. For example, the percentage that the amino acid C appears at position 1 in the sample of 500 nucleic acids was calculated. The values represent an approximate distribution in a population. Using a sufficient number of nucleic acids in the population, the sample distribution was close to the preselected distribution.

TABLE 8
Preselected Distribution of Amino Acids

| | Position | | | | | | | | | |
|---|---|---|---|---|---|---|---|---|---|---|
| Amino acid | 1 | 2 | 3 | 4 | 5 | 6 | 7 | 8 | 9 | 10 |
| % C | 0.1 | 0.2 | 0.1 | 0.1 | 0.0 | 0.0 | 0.1 | 6.0 | 2.0 | 0.1 |
| % A | 13.7 | 2.4 | 4.0 | 8.9 | 4.8 | 7.1 | 5.1 | 6.0 | 5.1 | 13.1 |
| % R | 13.7 | 16.7 | 6.7 | 13.3 | 6.0 | 8.3 | 5.1 | 6.0 | 3.8 | 6.6 |
| % N | 1.1 | 2.4 | 2.7 | 4.4 | 2.4 | 3.6 | 6.3 | 6.0 | 3.8 | 4.9 |
| % D | 15.8 | 16.7 | 5.3 | 4.4 | 1.2 | 13.1 | 21.5 | 6.0 | 11.4 | 8.2 |
| % Q | 7.4 | 6.0 | 4.0 | 2.2 | 1.2 | 1.2 | 2.5 | 6.0 | 3.8 | 1.6 |
| % E | 4.2 | 3.6 | 1.3 | 6.7 | 2.4 | 13.1 | 5.1 | 6.0 | 11.4 | 3.3 |
| % G | 14.7 | 15.5 | 20.0 | 13.3 | 27.4 | 9.5 | 11.4 | 6.0 | 11.4 | 3.3 |
| % H | 1.1 | 1.2 | 1.3 | 2.2 | 1.2 | 2.4 | 2.5 | 6.0 | 3.8 | 3.3 |
| % I | 1.1 | 1.2 | 10.7 | 3.3 | 1.2 | 6.0 | 5.1 | 6.0 | 3.8 | 3.3 |
| % L | 7.4 | 2.4 | 2.7 | 4.4 | 3.6 | 4.8 | 6.3 | 6.0 | 3.8 | 3.3 |
| % K | 2.1 | 4.8 | 1.3 | 2.2 | 6.0 | 1.2 | 1.3 | 6.0 | 2.5 | 9.8 |
| % M | 2.1 | 1.2 | 1.3 | 2.2 | 2.4 | 1.2 | 1.3 | 6.0 | 10.1 | 2.0 |
| % F | 1.1 | 1.2 | 4.0 | 6.7 | 3.6 | 2.4 | 1.3 | 6.0 | 2.5 | 3.3 |
| % P | 7.4 | 6.0 | 8.0 | 12.2 | 3.6 | 4.8 | 2.5 | 6.0 | 10.1 | 16.4 |
| % S | 5.3 | 9.5 | 10.7 | 10.0 | 19.0 | 19.0 | 19.0 | 6.0 | 8.9 | 11.5 |
| % T | 2.1 | 9.5 | 16.0 | 3.3 | 14.3 | 2.4 | 3.8 | 6.0 | 3.8 | 6.6 |

TABLE 9
Cumulative Normalization Distribution

| | Position | | | | | | | | | |
|---|---|---|---|---|---|---|---|---|---|---|
| Amino Acid | 1 | 2 | 3 | 4 | 5 | 6 | 7 | 8 | 9 | 10 |
| C | .00 | .00 | .00 | .00 | .00 | .00 | .00 | .06 | .02 | .00 |
| A | .14 | .03 | .04 | .09 | .05 | .07 | .05 | .12 | .07 | .13 |
| R | .27 | .19 | .11 | .22 | .11 | .15 | .10 | .18 | .11 | .20 |
| N | .28 | .22 | .13 | .27 | .13 | .19 | .17 | .24 | .14 | .25 |
| D | .44 | .38 | .19 | .31 | .14 | .32 | .38 | .29 | .26 | .33 |
| Q | .52 | .44 | .23 | .33 | .16 | .33 | .41 | .35 | .29 | .34 |
| E | .56 | .48 | .24 | .40 | .18 | .46 | .46 | .41 | .40 | .38 |
| G | .70 | .63 | .44 | .53 | .45 | .56 | .57 | .47 | .52 | .41 |
| H | .72 | .64 | .45 | .56 | .46 | .58 | .59 | .53 | .55 | .44 |
| I | .73 | .66 | .56 | .59 | .48 | .64 | .65 | .59 | .59 | .47 |
| L | .80 | .68 | .59 | .63 | .51 | .69 | .71 | .65 | .63 | .51 |
| K | .82 | .73 | .60 | .66 | .57 | .70 | .72 | .71 | .65 | .60 |
| M | .84 | .74 | .61 | .68 | .60 | .71 | .73 | .76 | .75 | .62 |
| F | .85 | .75 | .65 | .74 | .63 | .74 | .75 | .82 | .78 | .66 |
| P | .93 | .81 | .73 | .87 | .67 | .79 | .77 | .88 | .88 | .82 |
| S | .98 | .91 | .84 | .97 | .86 | .98 | .96 | .94 | .96 | .93 |
| T | 1 | 1 | 1 | 1 | 1 | 1 | 1 | 1 | 1 | 1 |

### Example 14. Generation of Nucleic Acid Libraries by Filtered Sampling

Using methods described in Example 13, re-sampling of the population was performed to remove undesired combinations and filter them out of the population. For example, a combination having 4 “H,” (histidine) amino acids at any position was deemed unfit for biological purposes. Accordingly, in this instance, when the 500th oligonucleotide was generated as “HHHCCHHCHH (SEQ ID NO: 55),” the combination was undesired due to having 8 H's. As a result, another randomly generated combination was generated in its place following the methods described in Example 13. Any number of criteria were used to generate a preselected distribution. For example, a population was generated to include at least one “A” (alanine) amino acid in each oligonucleotide at any position. A population was also generated such that no generated combination would have two “M” (methionine) amino acids adjacent to each other. Thus, random sampling was performed until a preselected distribution and particular criteria were met.

### Example 15: Combinatorial Libraries Having Uniform Distribution

De novo polynucleotide synthesis was performed under conditions similar to those described in Example 2. A nucleic acid population was generated as in Examples 4-6 and 8-12, encoding for codon variation at a single site or multiple sites where variants were preselected at each position and have a preselected distribution.

To generate a uniform variant distribution library by combinatorial methods, a reference sequence for the variant library was split into two portions. Uniform variant distribution as used herein is meant to mean that each variant is intended to be synthesized in approximately equal amounts. One side of the split was referred to as the 5′ side and the second side of the split was referred to as the 3′ side. Sequences were designed and synthesized for each side of the reference sequence such that, when annealed the desired nucleic acid library was synthesized. For a uniform library with variation similar to Table 10, the diversity on the 5′ side is 2548 (14×14×13). On the 3′ side, the diversity is 546 (3×13×14). The 5′ side and the 3′ side were synthesized by annealing, resulting in total diversity of 1,391,208 (2548×546). The variants were analyzed by next generation sequencing (data not shown).

TABLE 10
Variation of Uniform Library

| | |
|---|---|
| 5′ Variants | 3′ Variants |

| | | | | | | |
|---|---|---|---|---|---|---|
| N | N | N | N | | N | N |
| P | | | | | | |
| Q | | | | | | |
| R | R | R | R | | R | R |
| S | S | S | S | | S | S |
| T | | | | | | |
| V | V | V | V | | V | V |
| W | W | W | W | W | W | W |
| Y | Y | Y | Y | | Y | Y |
| A | A | A | A | A | A | A |
| C | | | | | | |
| D | | | | | | |
| E | | | | | | |
| F | F | F | F | | F | F |
| G | G | G | G | | G | G |
| H | H | H | H | | H | H |
| I | I | I | | | | I |
| K | K | K | K | K | K | K |
| L | L | L | L | | L | L |
| M | M | M | M | | M | M |

| | | |
|---|---|---|
| Diversity | 2548 | 546 |

| | |
|---|---|
| Total | 1391208 |
| Diversity | |

### Example 16: Combinatorial Libraries Having Non-Uniform Distribution

De novo polynucleotide synthesis was performed under conditions similar to those described in Example 2. A nucleic acid population was generated as in Examples 4-6 and 8-12, encoding for codon variation at a single site or multiple sites where variants were preselected at each position and have a preselected distribution.

A library with non-uniform variant distribution was also generated with a preselected distribution similar to what is seen in Table 11. A reference sequence was again, split in half and variants were generated for each portion. One side of the split was referred to as the 5′ side and the second side of the split was referred to as the 3′ side. The expected probabilities of the 5′ variants and the 3′ variants were calculated by multiplying the theoretical frequency of the substitution for that variant. For example, for a 5′ variant of sequence NRS, the expected probability was 0.0677% (9.9%×7.6%×9.0%). For the 5′ variants and for the 3′ variants, some of the variants had the same probabilities and were grouped together i.e. in the same probability “bin.” Thus, all variants within the same bin have the same theoretical frequency of occurring. For the 1,391,208 total theoretical variants, there were 162 different probabilities and thus 162 different probability bins.

TABLE 11
Variation Distribution

| | 5′ Variants | | | 3′ Variants | | |
|---|---|---|---|---|---|---|
| N | 9.9% | 7.6% | 8.7% | 0.0% | 8.7% | 9.9% |
| P | 0.0% | 0.0% | 0.0% | 0.0% | 0.0% | 0.0% |
| Q | 0.0% | 0.0% | 0.0% | 0.0% | 0.0% | 0.0% |
| R | 9.9% | 7.6% | 8.7% | 0.0% | 8.7% | 9.9% |
| S | 3.0% | 9.0% | 9.0% | 0.0% | 9.0% | 3.0% |
| T | 0.0% | 0.0% | 0.0% | 0.0% | 0.0% | 0.0% |
| V | 9.9% | 7.6% | 8.7% | 0.0% | 8.7% | 9.9% |
| W | 4.0% | 4.0% | 4.0% | 20.0% | 4.0% | 4.0% |
| Y | 9.9% | 7.6% | 8.7% | 0.0% | 8.7% | 9.9% |
| A | 4.0% | 4.0% | 4.0% | 60.0% | 4.0% | 4.0% |
| C | 0.0% | 0.0% | 0.0% | 0.0% | 0.0% | 0.0% |
| D | 0.0% | 0.0% | 0.0% | 0.0% | 0.0% | 0.0% |
| E | 0.0% | 0.0% | 0.0% | 0.0% | 0.0% | 0.0% |
| F | 9.9% | 7.6% | 8.7% | 0.0% | 8.7% | 9.9% |
| G | 9.9% | 7.6% | 8.7% | 0.0% | 8.7% | 9.9% |
| H | 9.9% | 7.6% | 8.7% | 0.0% | 8.7% | 9.9% |
| I | 9.9% | 7.6% | 0.0% | 0.0% | 0.0% | 9.9% |
| K | 4.0% | 4.0% | 4.0% | 20.0% | 4.0% | 4.0% |
| L | 3.0% | 9.0% | 9.0% | 0.0% | 9.0% | 3.0% |
| M | 3.0% | 9.0% | 9.0% | 0.0% | 9.0% | 3.0% |

Next generation sequencing (NGS) was then performed to determine how much of the theoretical diversity was represented in the variants generated. Because sequencing was performed with 10̂6 reads, only 30% of the actual diversity was observed. Thus, the total of the actual diversity represented at the desired frequency was determined.

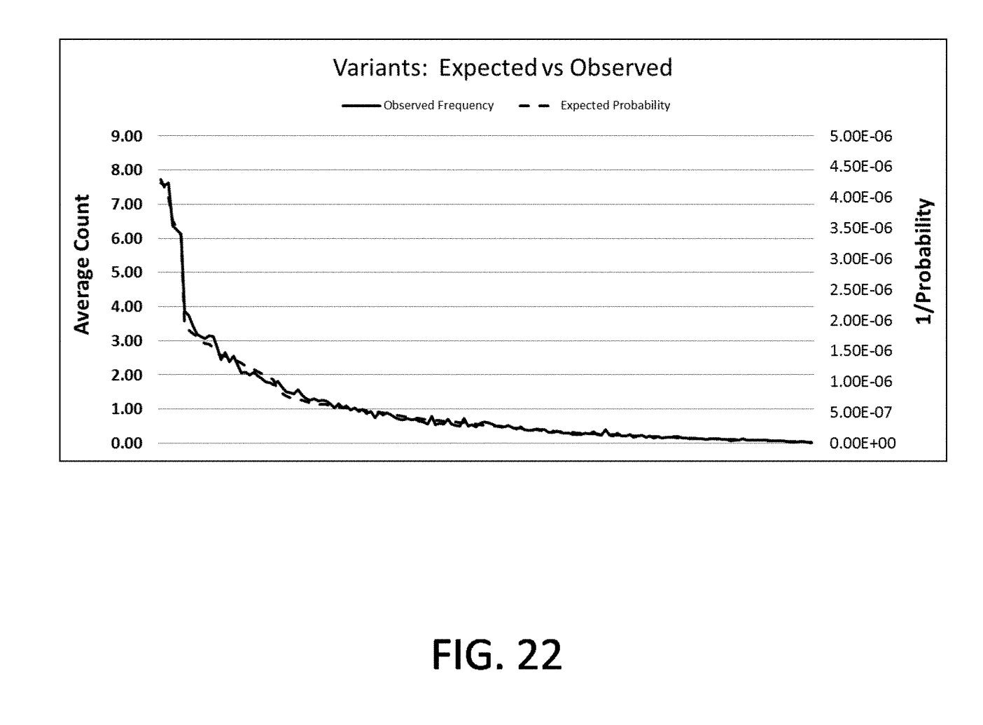

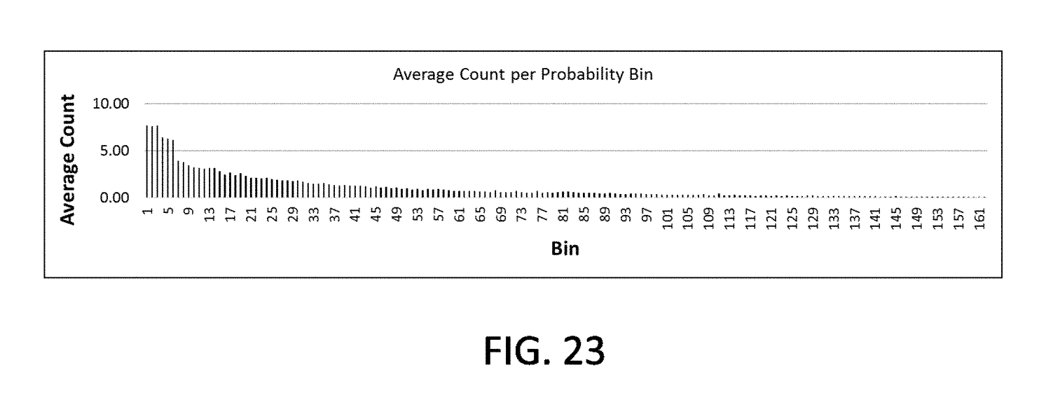

The 162 different probability bins, representing the number of variants with the same frequency, were used to analyze the NGS data. For the 162 different probability bins, reads from NGS were grouped by their expected probability of occurrence (dashed line) as seen in FIG. 22. Observed frequency (solid line) was then compared to the expected probability. For each of the 162 bins, the observed frequency was determined by the total number of variants divided by the number of variants in that bin. This value was calculated for each bin and is represented as the average count as seen in FIG. 23. These values were graphed as the observed frequency and compared to the expected probability as seen in FIG. 22.

Comparison of the observed frequency of variants (solid line) with the expected probability of variants (dashed line) as in FIG. 22 indicate whether the observed diversity was represented at the desired frequency. As seen in FIG. 22, the observed diversity matches well with the expected probability, and more than 99% of the theoretical diversity was represented.

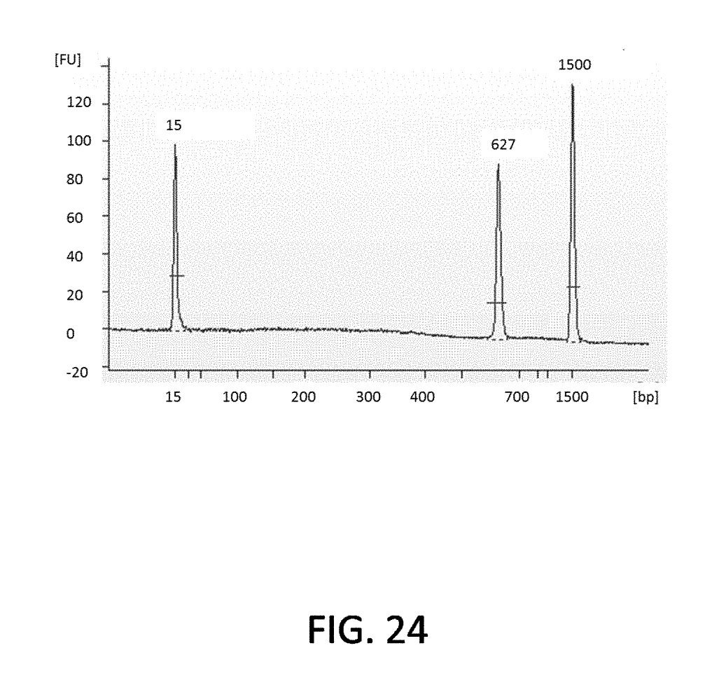

In addition, high frequency combinations were observed as well as the predetermined low-frequency combinations. 89.9% of the NGS reads spanning the 39 base pair region of diversity were the correct size and more than 70% of the complete 126 base pair construct was estimated to be insertion and deletion free. Referring to FIG. 24, a high percentage of full length fragments were generated as indicated by single peaks.

### Example 17: Combinatorial Library Comprising 144 Single Codon Variants and 9072 Double Codon Variants at Each of 8 Positions

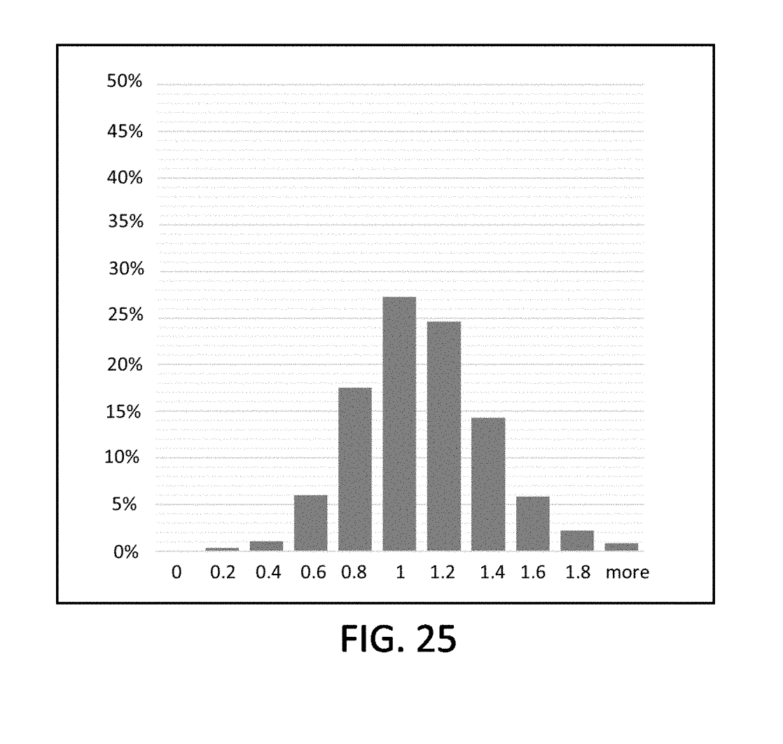

De novo polynucleotide synthesis was performed under conditions similar to those described in Example 2. A nucleic acid population was generated similarly to Examples 4-6 and 8-12. The nucleic acid population comprised 144 single codon variants and 9072 double codon variants (diversity of 9216) where variants were preselected at 8 positions. Next generation sequencing (NGS) was then performed to determine a distribution of observed combinatorial variants. Sequencing was performed with more than 10̂5 read coverage. As seen in FIG. 25, more than 99% of observed variants were detected by NGS with a uniform distribution. Greater than 90% of the observed variants were insertion and deletion free, and less than 5% off target sequences were detected. Less than 1% of wild-type sequences was observed.

### Example 18: Generation of Representative Variant Libraries Using Array-Based Methods

A variant library was de novo synthesized using an array-based method similar to Examples 1-3. The variant library generated using an array-based method was then compared to a variant library generated using a PCR-based method.

Following variant library construction, colonies from the two libraries were sampled and sequenced. The data is shown in Table 12. The number of failed sequencing (“No. of failed sequencing”) was determined as the number of colonies in which sequencing was not possible. The percentage diversity (Diversity (%)) was determined from the ratio of the number of mutants obtained after sequencing to the number of theoretically possible mutants expected. The percentage correctness (“Correctness (%)) was determined by the ratio of the number of mutants with correct DNA sequences to the number of mutants used for sequencing. From Table 12, the variant library generated using an array-based method demonstrated higher “correctness,” correlating with improved diversity and quality.

The two libraries were also compared on the protein level by sampling. The variant library generated using an array-based method had a more representative variant population with increased number of theoretically expected mutants generated than the variant library generated using a PCR-based method.

TABLE 12
Variant Library Data

| | No. of | No. of | No. of | | No. of | | |
|---|---|---|---|---|---|---|---|
| | mutants with | mutants with | failed | No. of | different | Diversity | Correctness |
| Libraries | deletions | insertions | sequencing | WT | mutants | (%) | (%) |
| PCR-based | 14 | 4 | 18 | 4 | 109 | 42.5 | 86.4 |
| Library | | | | | | | |
| Array-based | 12 | 4 | 2 | 2 | 164 | 64.1 | 93.2 |
| Library | | | | | | | |

### Example 19: Codon Assignment Scheme

A polynucleotide library was designed using a codon assignment. The codon assignment was used to determine the codon sequence to be designed at each site.

Codon variation was generated for the human tumor protein p53 (TP53) having a wild-type (WT) amino acid sequence and WT DNA sequence as listed in Table 13. When generating codon variation, the variant codon sequence to be designed was based on the Codon Assignment of Table 3 above. Specifically, when generating a variant amino acid from the WT amino acid, the variant codon sequence encoding the variant amino acid was chosen first from left to right from the codon sequences listed in Table 3.

Referring to Table 13, the WT amino acid at position 2 of the peptide is “F” (in bold). To generate variation at position 2, variants of the WT sequence were designed in which “F” was changed to any of the other 19 amino acids. The Codon Assignment according to Table 3 was then used to determine which variant codon sequence to design to generate a variant amino acid at that position. To generate a variant in which “F” is changed to “A,” the variant codon sequence that was chosen first according to Table 3 was “GCT” instead of “GCA,” “GCC,” or “GCG,” which all encode for “A.” Table 14 lists all the possible variant amino acids of “F” at position 2 and which variant codon sequence was designed to generate the variant amino acid.

TABLE 13
Sequences for Variation

| | Amino | |
|---|---|---|
| SEQ | Acid or | |
| ID NO | DNA | SEQUENCE |
| 33 | Amino Acid | MFCQLAKTCPVQLWVDSTPPPGTRVRAMAIYKQSQHMTEVVRRCPH |
| | | HERCSDSDGLAPPQHLIRVEGNLRVEYLDDRNTFRHSVVVPYEPPEVG |
| | | SDCTTIHYNYMCNSSCMGGMNRRPILTIITLEDSSGNLLGRNSFEVRVC |
| | | ACPGRDRRTEEENLRKKGEPHHELPPGSTKRALPNNTSSSPQPKKKPL |
| | | DGEYFTLQIRGRERFEMFRELNEALELKDAQAGKEPGGSRAHSSHLKS |
| | | KKGQSTSRHKKLMFKTEGPDSD |
| 34 | DNA | TGAGGCCAGGAGATGGAGGCTGCAGTGAGCTGTGATCACACCACT |
| | | GTGCTCCAGCCTGAGTGACAGAGCAAGACCCTATCTCAAAAAAAA |
| | | AAAAAAAAAAGAAAAGCTCCTGAGGTGTAGACGCCAACTCTCTCT |
| | | AGCTCGCTAGTGGGTTGCAGGAGGTGCTTACGCATGTTTGTTTCTTT |
| | | GCTGCCGTCTTCCAGTTGCTTTATCTGTTCACTTGTGCCCTGACTTT |
| | | CAACTCTGTCTCCTTCCTCTTCCTACAGTACTCCCCTGCCCTCAACA |
| | | AGATGTTTTGCCAACTGGCCAAGACCTGCCCTGTGCAGCTGTGGGT |
| | | TGATTCCACACCCCCGCCCGGCACCCGCGTCCGCGCCATGGCCATC |
| | | TACAAGCAGTCACAGCACATGACGGAGGTTGTGAGGCGCTGCCCC |
| | | CACCATGAGCGCTGCTCAGATAGCGATGGTCTGGCCCCTCCTCAGC |
| | | ATCTTATCCGAGTGGAAGGAAATTTGCGTGTGGAGTATTTGGATGA |
| | | CAGAAACACTTTTCGACATAGTGTGGTGGTGCCCTATGAGCCGCCT |
| | | GAGGTTGGCTCTGACTGTACCACCATCCACTACAACTACATGTGTA |
| | | ACAGTTCCTGCATGGGCGGCATGAACCGGAGGCCCATCCTCACCAT |
| | | CATCACACTGGAAGACTCCAGTGGTAATCTACTGGGACGGAACAG |
| | | CTTTGAGGTGCGTGTTTGTGCCTGTCCTGGGAGAGACCGGCGCACA |
| | | GAGGAAGAGAATCTCCGCAAGAAAGGGGAGCCTCACCACGAGCTG |
| | | CCCCCAGGGAGCACTAAGCGAGCACTGCCCAACAACACCAGCTCC |
| | | TCTCCCCAGCCAAAGAAGAAACCACTGGATGGAGAATATTTCACC |
| | | CTTCAGATCCGTGGGCGTGAGCGCTTCGAGATGTTCCGAGAGCTGA |
| | | ATGAGGCCTTGGAACTCAAGGATGCCCAGGCTGGGAAGGAGCCAG |
| | | GGGGGAGCAGGGCTCACTCCAGCCACCTGAAGTCCAAAAAGGGTC |
| | | AGTCTACCTCCCGCCATAAAAAACTCATGTTCAAGACAGAAGGGC |
| | | CTGACTCAGACTGACATTCTCCACTTCTTGTTCCCCACTGACAGCCT |
| | | CCCACCCCCATCTCTCCCTCCCCTGCCATTTTGGGTTTTGGGTCTTT |
| | | GAACCCTTGCTTGCAATAGGTGTGCGTCAGAAGCACCCAGGACTTC |
| | | CATTTGCTTTGTCCCGGGGCTCCACTGAACAAGTTGGCCTGCACTG |
| | | GTGTTTTGTTGTGGGGAGGAGGATGGGGAGTAGGACATACCAGCT |
| | | TAGATTTTAAGGTTTTTACTGTGAGGGATGTTTGGGAGATGTAAGA |
| | | AATGTTCTTGCAGTTAAGGGTTAGTTTACAATCAGCCACATTCTAG |
| | | GTAGGGGCCCACTTCACCGTACTAACCAGGGAAGCTGTCCCTCACT |
| | | GTTGAATTTTCTCTAACTTCAAGGCCCATATCTGTGAAATGCTGGC |
| | | ATTTGCACCTACCTCACAGAGTGCATTGTGAGGGTTAATGAAATAA |
| | | TGTACATCTGGCCTTGAAACCACCTTTTATTACATGGGGTCTAGAA |
| | | CTTGACCCCCTTGAGGGTGCTTGTTCCCTCTCCCTGTTGGTCGGTGG |
| | | GTTGGTAGTTTCTACAGTTGGGCAGCTGGTTAGGTAGAGGGAGTTG |
| | | TCAAGTCTCTGCTGGCCCAGCCAAACCCTGTCTGACAACCTCTTGG |
| | | TGAACCTTAGTACCTAAAAGGAAATCTCACCCCATCCCACACCCTG |
| | | GAGGATTTCATCTCTTGTATATGATGATCTGGATCCACCAAGACTT |
| | | GTTTTATGCTCAGGGTCAATTTCTTTTTTCTTTTTTTTTTTTTTTTTTC |
| | | TTTTTCTTTGAGACTGGGTCTCGCTTTGTTGCCCAGGCTGGAGTGGA |
| | | GTGGCGTGATCTTGGCTTACTGCAGCCTTTGCCTCCCCGGCTCGAG |
| | | CAGTCCTGCCTCAGCCTCCGGAGTAGCTGGGACCACAGGTTCATGC |
| | | CACCATGGCCAGCCAACTTTTGCATGTTTTGTAGAGATGGGGTCTC |
| | | ACAGTGTTGCCCAGGCTGGTCTCAAACTCCTGGGCTCAGGCGATCC |
| | | ACCTGTCTCAGCCTCCCAGAGTGCTGGGATTACAATTGTGAGCCAC |
| | | CACGTCCAGCTGGAAGGGTCAACATCTTTTACATTCTGCAAGCACA |
| | | TCTGCATTTTCACCCCACCCTTCCCCTCCTTCTCCCTTTTTATATCCC |
| | | ATTTTTATATCGATCTCTTATTTTACAATAAAACTTTGCTGCCACCT |
| | | GTGTGTCTGAGGGGTG |

TABLE 14
Variant Amino Acids

| | WT | | Variant | | | Amino | |
|---|---|---|---|---|---|---|---|
| SEQ | Amino | WT | Amino | Variant | DNA | Acid | Example subsequence (variant |
| ID NO | Acid | Codon | Acid | Codon | Position | Position | codon in lowercase) |
| 35 | F | TTT | A | GCT | 282 | 2 | CCCCTGCCCTCAACAAGATG |
| | | | | | | | gctTGCCAACTGGCCAA |
| 36 | F | TTT | C | TGC | 282 | 2 | CCCCTGCCCTCAACAAGATG |
| | | | | | | | tgcTGCCAACTGGCCAA |
| 37 | F | TTT | D | GAT | 282 | 2 | CCCCTGCCCTCAACAAGATG |
| | | | | | | | gatTGCCAACTGGCCAA |
| 38 | F | TTT | E | GAG | 282 | 2 | CCCCTGCCCTCAACAAGATG |
| | | | | | | | gagTGCCAACTGGCCAA |
| 39 | F | TTT | F | TTC | 282 | 2 | CCCCTGCCCTCAACAAGATG |
| | | | | | | | ttcTGCCAACTGGCCAA |
| 40 | F | TTT | G | GGT | 282 | 2 | CCCCTGCCCTCAACAAGATG |
| | | | | | | | ggtTGCCAACTGGCCAA |
| 41 | F | TTT | H | CAC | 282 | 2 | CCCCTGCCCTCAACAAGATG |
| | | | | | | | cacTGCCAACTGGCCAA |
| 42 | F | TTT | I | ATC | 282 | 2 | CCCCTGCCCTCAACAAGATG |
| | | | | | | | atcTGCCAACTGGCCAA |
| 43 | F | TTT | K | AAG | 282 | 2 | CCCCTGCCCTCAACAAGATG |
| | | | | | | | aagTGCCAACTGGCCAA |
| 44 | F | TTT | L | CTG | 282 | 2 | CCCCTGCCCTCAACAAGATG |
| | | | | | | | ctgTGCCAACTGGCCAA |
| 45 | F | TTT | M | ATG | 282 | 2 | CCCCTGCCCTCAACAAGATG |
| | | | | | | | atgTGCCAACTGGCCAA |
| 46 | F | TTT | N | AAC | 282 | 2 | CCCCTGCCCTCAACAAGATG |
| | | | | | | | aacTGCCAACTGGCCAA |
| 47 | F | TTT | P | CCT | 282 | 2 | CCCCTGCCCTCAACAAGATG |
| | | | | | | | cctTGCCAACTGGCCAA |
| 48 | F | TTT | Q | CAG | 282 | 2 | CCCCTGCCCTCAACAAGATG |
| | | | | | | | cagTGCCAACTGGCCAA |
| 49 | F | TTT | R | AGA | 282 | 2 | CCCCTGCCCTCAACAAGATG |
| | | | | | | | agaTGCCAACTGGCCAA |
| 50 | F | TTT | S | AGC | 282 | 2 | CCCCTGCCCTCAACAAGATG |
| | | | | | | | agcTGCCAACTGGCCAA |
| 51 | F | TTT | T | ACC | 282 | 2 | CCCCTGCCCTCAACAAGATG |
| | | | | | | | accTGCCAACTGGCCAA |
| 52 | F | TTT | V | GTG | 282 | 2 | CCCCTGCCCTCAACAAGATG |
| | | | | | | | gtgTGCCAACTGGCCAA |
| 53 | F | TTT | W | TGG | 282 | 2 | CCCCTGCCCTCAACAAGATG |
| | | | | | | | tggTGCCAACTGGCCAA |
| 54 | F | TTT | Y | TAC | 282 | 2 | CCCCTGCCCTCAACAAGATG |
| | | | | | | | tacTGCCAACTGGCCAA |

### Example 20: Stretch in a CDR Having Multiple Variant Sites

A nucleic acid library is generated as in Examples 4-6 and 8-12, encoding for codon variation at a single site or multiple sites where variants are preselected at each position. The variant region encodes for at least a portion of a CDR. See, for example, FIG. 12. Synthesized nucleic acids are released from the device surface, and used as primers to generate a nucleic acid library, which is expressed in cells to generate a variant protein library. Variant antibodies are assessed for increase binding affinity to an epitope.

### Example 21: Generation of Variant Antibody Libraries

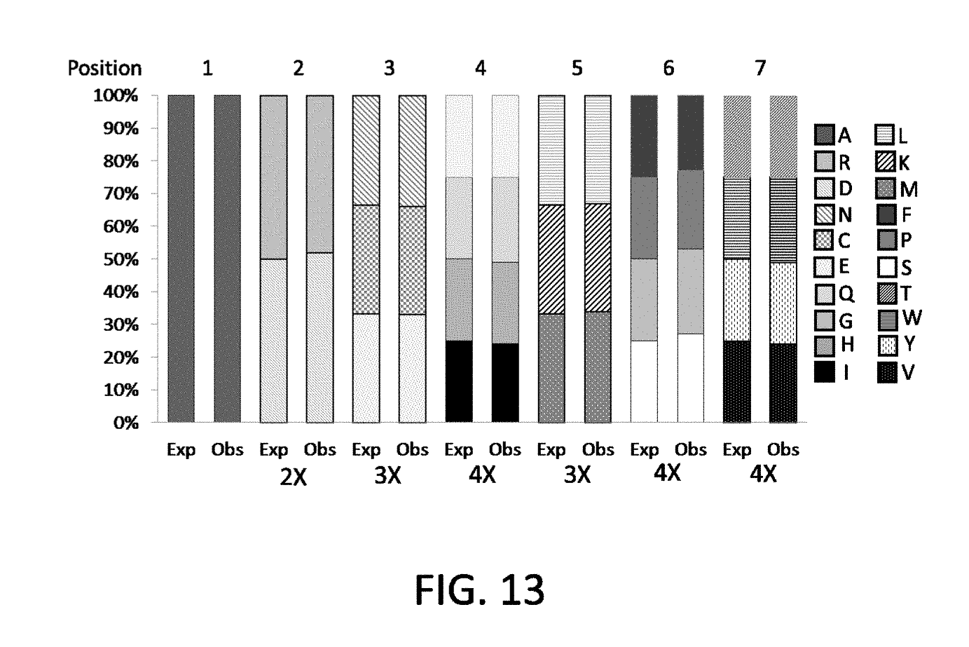

A nucleic acid library is generated as in the Examples above. A variant library is generated for nucleic acids encoding for a representative CDR from FIG. 12. The representative CDR is modified where the CDR region comprises multiple positions for variation as seen in FIG. 13. As shown in FIG. 13, a different number of codon variants and the positions of the variants are selected. In FIG. 13, the diversity of variant libraries that can be created is 1,152. Analysis by next generation sequencing demonstrates the presence of the intended variants at the right fraction and at the right position.

### Example 22: Modular Plasmid Components for Expressing Diverse Peptides

A nucleic acid library is generated as in Examples 4-6 and 8-12, encoding for codon variation at a single site or multiple sites for each of separate regions that make up potions of an expression construct cassette, as depicted in FIG. 14. To generate a two construct expressing cassette, variant nucleic acids were synthesized encoding at least a portion of a variant sequence of a first promoter 1410, first open reading frame 1420, first terminator 1430, second promoter 1440, second open reading frame 1450, or second terminator sequence 1460. After rounds of amplification, as described in previous examples, a library of 1,024 expression constructs is generated.

### Example 23: Multiple Site, Single Position Variants

A nucleic acid library is generated as in Examples 4-6 and 8-12, encoding for codon variation at a single site or multiple sites in a region encoding for at least a portion of nucleic acid. A library of nucleic acid variants is generated, wherein the library consists of multiple site, single position variants. See, for example, FIG. 8B.

### Example 24: Variant Library Synthesis

De novo polynucleotide synthesis is performed under conditions similar to those described in Example 2. At least about 30,000 non-identical polynucleotides are de novo synthesized, wherein each of the non-identical polynucleotides encodes for a different codon variant of an amino acid sequence. The synthesized at least 30,000 non-identical polynucleotides have an aggregate error rate of less than 1 in 1:000 bases compared to predetermined sequences for each of the at least about 30,000 non-identical polynucleotides. The library is used for PCR mutagenesis of a long nucleic acid and at least about 30,000 non-identical variant polynucleotides are formed.

### Example 25: Cluster-Based Variant Library Synthesis

De novo polynucleotide synthesis is performed under conditions similar to those described in Example 2. A single cluster on a device is generated which contained synthesized predetermined variants of a reference nucleic acid for 2 codon positions. In the 2 consecutive codon position arrangement, 19 variants/per position were generated for the 2 positions with 2 replicates of each nucleic acid, and resulted in 38 nucleic acids synthesized. Each variant sequence is 40 bases in length. In the same cluster, additional non-variant nucleic acid sequences are generated, where the additional non-variant nucleic acids and the variant nucleic acids collective encode for 38 variants of the coding sequence of a gene. Each of the nucleic acids has at least one region complementary to another of the nucleic acids. The nucleic acids in the cluster are released by gaseous ammonia cleavage. A pin comprising water contacts the cluster, picks up the nucleic acids, and moves the nucleic acids to a small vial. The vial also contains DNA polymerase reagents for a polymerase cycling assembly (PCA) reaction. The nucleic acids anneal, gaps are filled in by an extension reaction, and resultant double-stranded DNA molecules are formed, forming a variant nucleic acid library. The variant nucleic acid library is, optionally, subjected to restriction enzyme is then ligated into expression vectors.

### Example 26: Screening a Variant Nucleic Acid Library for Changes in Protein Binding Affinity

A plurality of expression vectors is generated as described in Examples 13-16. In this example, the expression vector is a HIS-tagged bacterial expression vector. The vector library is electroporated into bacterial cells and then clones are selected for expression and purification of HIS-tagged variant proteins. The variant proteins are screened for a change binding affinity to a target molecule.

Affinity is examined by methods such as using metal affinity chromatography (IMAC), where a metal ion coated resin (e.g., IDA-agarose or NTA-agarose) is used to isolate HIS-tagged proteins. Expressed His-tagged proteins can be purified and detected because the string of histidine residues binds to several types of immobilized metal ions, including nickel, cobalt and copper, under specific buffer conditions. An example binding/wash buffer consists of Tris-buffer saline (TBS) pH 7.2, containing 10-25 mM imidazole. Elution and recovery of captured HIS-tagged protein from an IMAC column is accomplished with a high concentration of imidazole (at least 200 mM) (the elution agent), low pH (e.g., 0.1M glycine-HCl, pH 2.5) or an excess of strong chelator (e.g., EDTA).

Alternatively, anti-HIS-tag antibodies are commercially available for use in assay methods involving HIS-tagged proteins, such as a pull-down assay to isolate HIS-tagged proteins or an immunoblotting assay to detect HIS-tagged proteins.

### Example 27: Screening a Variant Nucleic Acid Library for Changes in Activity for a Regulator of Cell Adhesion and Migration

A variant nucleic acid library generated as described in Examples 13-16 is inserted into a GFP-tagged mammalian expression vector. Isolated clones from the library are transiently transfected into mammalian cells. Alternatively, proteins are expressed and isolated from cells containing the expression constructs, and then the proteins are delivered to cells for further measurements Immunofluorescent assays are conducted to assess changes in cellular localization of the GFP-tagged variant expression products. FACS assays are conducted to assess changes in the conformational state of a transmembrane protein that interacts with a non-variant version of a GFP-tagged variant protein expression product. Wound healing assays are conducted to assess changes in the ability of cells expressing a GFP-tagged variant protein to invade space created by a scratch on a cell culture dish. Cells expressing GFP-tagged proteins are identified and tracked using a fluorescent light source and a camera.

### Example 28: Screening a Variant Nucleic Acid Library for Peptides Inhibiting Viral Progression

A variant nucleic acid library generated as described in Examples 13-16 is inserted into a FLAG-tagged mammalian expression vector and the variant nucleic acid library encodes for peptide sequences. Primary mammalian cells are obtained from a subject suffering from a viral disorder. Alternatively, primary cells from a healthy subject are infected with a virus. Cells are plated on a series of microwell dishes. Isolated clones from the variant library are transiently transfected into the cells. Alternatively, proteins are expressed and isolated from cells containing the expression constructs, and then the proteins are delivered to cells for further measurements. Cell survival assays are performed to assess infected cells for enhanced survival associated with a variant peptide. Exemplary viruses include, without limitation, avian flu, zika virus, Hantavirus, Hepatitis C, and smallpox.

One example assay is the neutral red cytotoxicity assay which uses neutral red dye, that, when added to cells, diffuses across the plasma membrane and accumulates in the acidic lysosomal compartment due to the mildly cationic properties of neutral red. Virus-induced cell degeneration leads to membrane fragmentation and loss of lysosome ATP-driven proton translocating activity. The consequent reduction of intracellular neutral red can be assessed spectrophotometrically in a multi-well plate format. Cells expressing variant peptides are scored by an increase in intracellular neutral red in a gain-of-signal color assay. Cells are assessed for peptides inhibiting virus-induced cell degeneration.

### Example 29: Screening for Variant Proteins that Increase or Decrease Metabolic Activity of a Cell

A plurality of expression vectors is generated as described in Examples 13-16 for the purpose of identifying expression products that result in a change in metabolic activity of a cell. In this example, the expression vectors are transferred (e.g., via transfection or transduction) into cells plated on a series of microwell dishes. Cells are then screened for one or more changes in metabolic activity. Alternatively, proteins are expressed and isolated from cells containing the expression constructs, and then the proteins are delivered to cells for measuring metabolic activity. Optionally, cells for measuring metabolic activity are treated with a toxin prior to screening for one or more changes metabolic activity. Exemplary toxins administered included, without limitation, botulinum toxin (including immunological types: A, B, C1, C2, D, E, F, and G), staphylococcus enterotoxin B, Yersinia pestis, Hepatitis C, Mustard agents, heavy metals, cyanide, endotoxin, Bacillus anthracia, zika virus, avian flu, herbicides, pesticides, mercury, organophosphates, and ricin.

The basal energy requirements are derived from the oxidation of metabolic substrates, e.g., glucose, either by oxidative phosphorylation involving the aerobic tricarboxylic acid (TCA) or Kreb's cycle or anaerobic glycolysis. When glycolysis is the major source of energy, the metabolic activity of cells can be estimated by monitoring the rate at which the cells excrete acidic products of metabolism, e.g., lactate and CO₂. In the case of aerobic metabolism, the consumption of extracellular oxygen and the production of oxidative free radicals are reflective of the energy requirements of the cell. Intracellular oxidation-reduction potential can be measured by autofluorescent measurement of the NADH and NAD⁺. The amount of energy, e.g., heat, released by the cell is derived from analytical values for substances produced and/or consumed during metabolism which under normal settings can be predicted from the amount of oxygen consumed (e.g., 4.8 kcal/l O₂). The coupling between heat production and oxygen utilization can be disturbed by toxins. Direct microcalorimetry measures the temperature rise of a thermally isolated sample. Thus, when combined with measurements of oxygen consumption calorimetry can be used to detect the uncoupling activity of toxins.

Various methods and devices are known in the art for measuring changes in various marker of metabolic activity. For example, such methods, devices, and markers are discussed in U.S. Pat. No. 7,704,745, which is herein incorporated by reference in its entirety. Briefly, measurement of the any of the following characteristics is recorded for each cell population: glucose, lactate, CO₂, NADH and NAD⁺ ratio, heat, O₂ consumption, and free-radical production. Cells screened can include hepatocyte, macrophages or neuroblastoma cells. Cells screened can be cell lines, primary cells from a subject, or cells from a model system (e.g., a mouse model).

Various techniques are available for measurement of the oxygen consumption rates of single cells or a population of cells located within a chamber of a multi-well plate. For example, chambers comprising the cells can have sensors for recording changes in temperature, current or fluorescence, as well as optical systems, e.g., a fiber-coupled optical system, coupled to each chamber to monitor fluorescent light. In this example, each chamber has a window for an illumination source to excite molecules inside the chamber. The fiber-coupled optical system can detect autofluorescence to measure intracellular NADH/NAD ratios and voltage and calcium-sensitive dyes to determine transmembrane potential and intracellular calcium. In addition, changes in CO₂ and/or O₂ sensitive fluorescent dye signal are detected.

### Example 30: Screening a Variant Nucleic Acid Library for Selective Targeting of Cancer Cells

A variant nucleic acid library generated as described in Examples 13-16 is inserted into a FLAG-tagged mammalian expression vector and the variant nucleic acid library encodes for peptide sequences. Isolated clones from the variant library are transiently transfected separately into cancer cells and non-cancer cells. Cell survival and cell death assays are performed on both the cancer and non-cancer cells, each expressing a variant peptide encoded by the variant nucleic acids. Cells are assessed for selective cancer cell killing associated with a variant peptide. The cancer cells are, optionally, a cancer cell line or primary cancer cells from a subject diagnosed with cancer. In the case of primary cancer cells from a subject diagnosed with cancer, a variant peptide identified in the screening assay is, optionally, selected for administration to the subject. Alternatively, proteins are expressed and isolated from cells containing the protein expression constructs, and then the proteins are delivered to cancer cells and non-cancer cells for further measurements.

### Example 31: Generation of a Combinatorial Library

De novo polynucleotide synthesis is performed under conditions generally described in Example 2. A nucleic acid population is generated as in Examples 4-6 and 8-12, encoding for codon variation at a single site or multiple sites where variants are preselected at each position. A combinatorial library is generated by combining nucleic acids of a first population with nucleic acids from a second population. As shown in shown in FIG. 1, a population of 4 nucleic acids 110 is combined with another population of 4 nucleic acids 120 to yield 16 combinations.

The nucleic acids are annealed by blunt end ligation. 50 ng of DNA of one nucleic acid is mixed with 50 ng of DNA of another nucleic acid in a 1.5 ml vial. Next, 1 μL of T4 DNA ligase (New England BioLabs) is added along with 20 μL of Ligation Buffer and 20 μL of nuclease-free water. The reaction mixture is then incubated. Following incubation, the ligation product is analyzed by sequencing.

### Example 32: Generation of a Combinatorial Library by Sampling

De novo polynucleotide synthesis is performed under conditions generally described in Example 2. A nucleic acid population is generated as in Examples 4-6 and 8-12, encoding for codon variation at a single site or multiple sites where variants are preselected at each position.

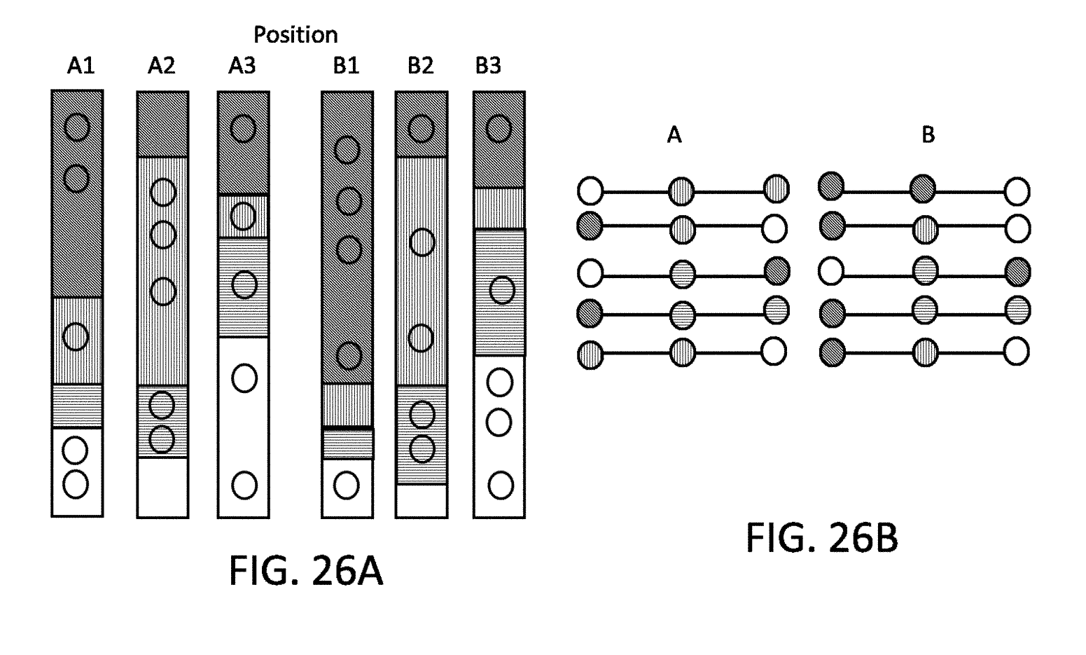

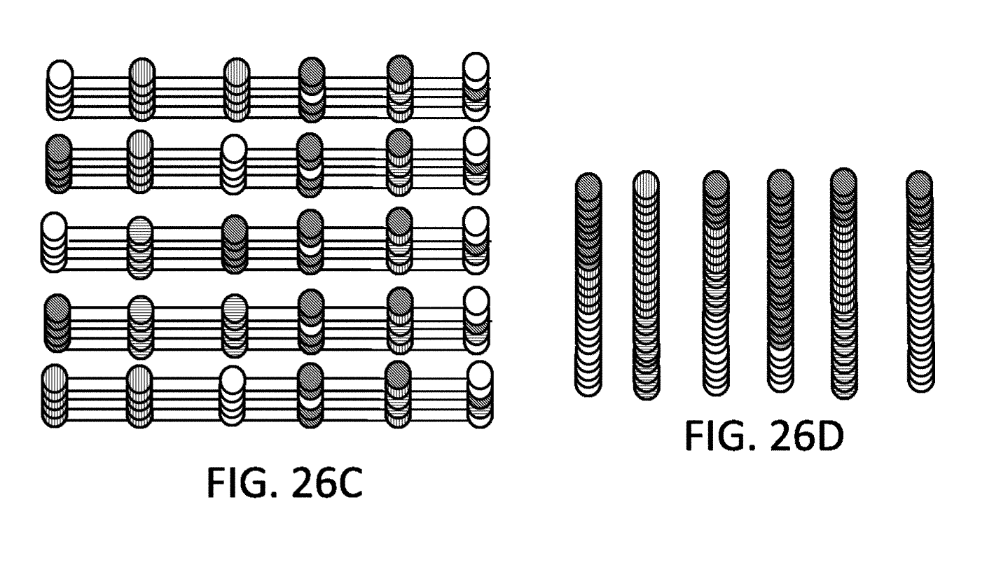

Referring to FIG. 26A, a library with non-uniform variant distribution is generated with a preselected distribution by similar methods described in Examples 13-16. Each patterned portion of the image represents 1 of 4 different amino acids with a different preselected distribution at each position (A1, A2, A3, B1, B2, and B3). The black circles represent random selections within each position. Referring to FIG. 26B, 5 randomly generated samples for A and 5 randomly generated samples for B are independently generated. The 5 randomly generated samples at A and the 5 randomly generated samples at B are then annealed together, for example as by blunt end ligation, as seen in FIG. 26C. This results in 25 combinations (n̂2=5̂2). Referring to FIG. 26D, statistical comparison demonstrates that resulting distribution aligns with the preselected distribution.

### Example 33: Generation of a Combinatorial Antibody Library

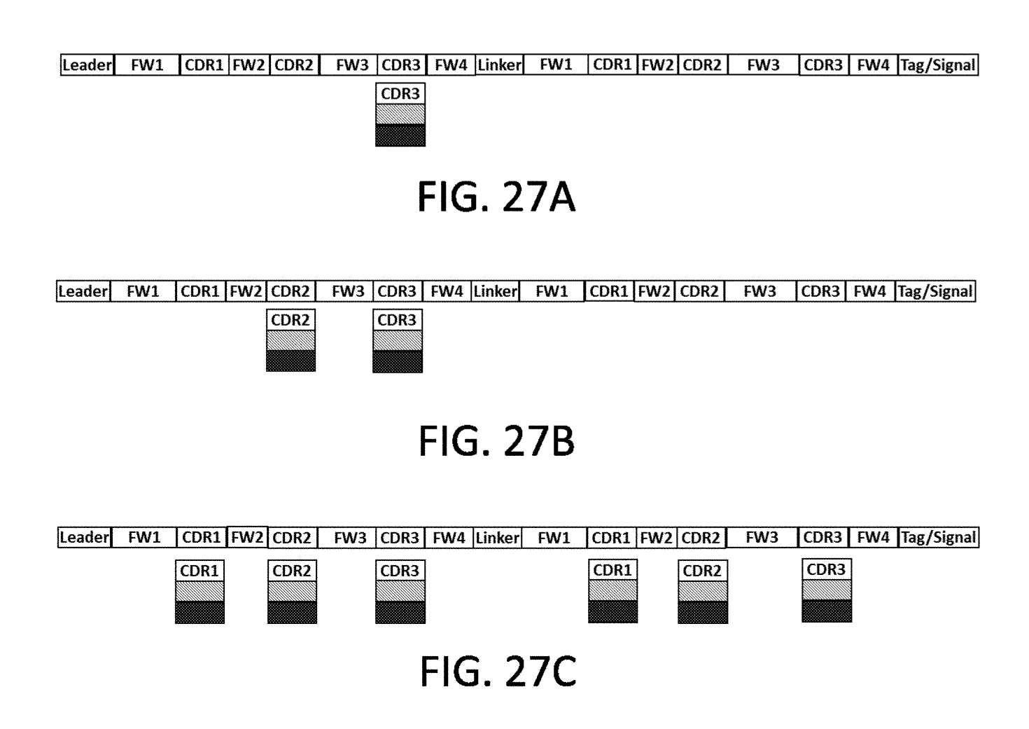

A nucleic acid library is generated as in the Examples above. A variant library is generated for nucleic acids encoding for a single CDR region as seen in FIG. 27A, two CDR regions as seen in FIG. 27B, or multiple CDR regions as seen in FIG. 27C.

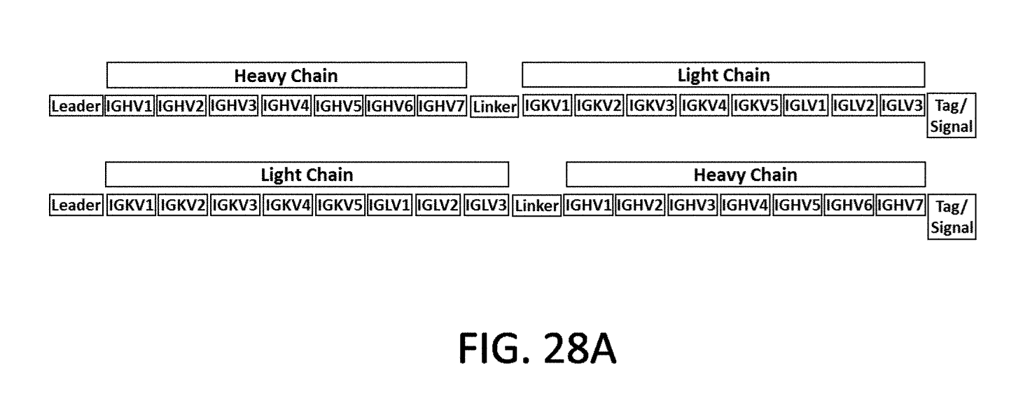

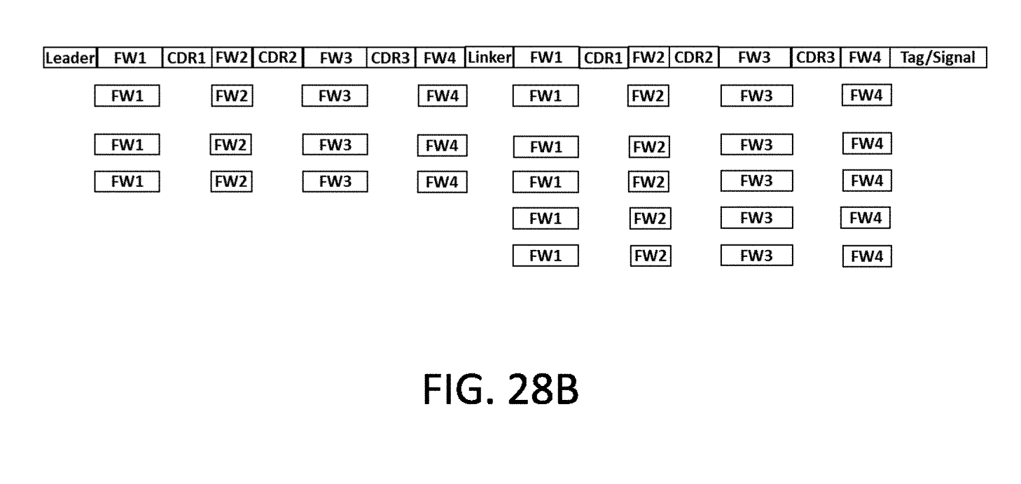

A variant antibody library is also generated to comprise variants in a single or multiple heavy and light chain scaffolds as seen in FIG. 28A or variants in a single or multiple frameworks as seen in FIG. 28B.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of synthesizing a variant nucleic acid library, comprising:
a. providing predetermined sequences encoding for at least 500 polynucleotide sequences, wherein the at least 500 polynucleotide sequences have a preselected codon distribution;
b. synthesizing a plurality of polynucleotides encoding for the at least 500 polynucleotide sequences;
c. assaying an activity for nucleic acids encoded by or proteins translated based on the plurality of polynucleotides; and
d. collecting results from the assay in step (c), wherein the collecting comprises collecting results of predetermined sequences associated with a negative or null result.

2. The method of claim 1, wherein step (d) comprises collecting results for at least 80% of the predetermined sequences.

3. (canceled)

4. (canceled)

5. The method of claim 1, wherein at least about 70% of a predicted diversity is represented.

6. (canceled)

7. (canceled)

8. (canceled)

9. The method of claim 1, wherein at least about 80% of the at least 500 polynucleotide sequences are each present in the variant nucleic acid library in an amount within 2× of a mean frequency for each of the polynucleotide sequences in the library.

10. (canceled)

11. The method of claim 1, wherein the activity is cellular activity.

12. The method of claim 11, wherein the cellular activity comprises reproduction, growth, adhesion, death, migration, energy production, oxygen utilization, metabolic activity, cell signaling, response to free radical damage, or any combination thereof.

13. The method of claim 1, wherein the variant nucleic acid library encodes sequences for variant genes or fragments thereof.

14. The method of claim 1, wherein the variant nucleic acid library encodes for at least a portion of an antibody, an enzyme, or a peptide.

15. A method for generating a combinatorial library of nucleic acids, the method comprising:
a. designing predetermined sequences encoding for:
i. a first plurality of polynucleotides, wherein each polynucleotide of the first plurality of polynucleotides encodes for a variant sequence compared to a single reference sequence and
ii. a second plurality of polynucleotides, wherein each polynucleotide of the second plurality of polynucleotides encodes for a variant sequence compared to the single reference sequence;
b. synthesizing the first plurality of polynucleotides and the second plurality of polynucleotides; and
c. mixing the first plurality of polynucleotides and the second plurality of polynucleotides to form the combinatorial library of nucleic acids, wherein at least about 70% of a predicted diversity is represented.

16. (canceled)

17. (canceled)

18. (canceled)

19. The method of claim 15, wherein the combinatorial library is a non-saturating combinatorial library, and wherein a total number of polynucleotides for generation of the non-saturating combinatorial library is at least 25% less than the total number polynucleotides for generation of a saturating combinatorial library.

20. (canceled)

21. (canceled)

22. (canceled)

23. (canceled)

24. The method of claim 15, wherein the combinatorial library when translated encodes for a protein library.

25. (canceled)

26. The method of claim 15, further comprising performing PCR mutagenesis of a nucleic acid using the combinatorial library as primers for a PCR mutagenesis reaction.

27. The method of claim 15, wherein the combinatorial library encodes sequences for variant genes or fragments thereof.

28. The method of claim 15, wherein the combinatorial library encodes for at least a portion of an antibody, an enzyme, or a peptide.

29. The method of claim 28, wherein the combinatorial library encodes for at least a portion of a variable region or a constant region of the antibody.

30. The method of claim 28, wherein the combinatorial library encodes for at least one CDR region of the antibody.

31. The method of claim 28, wherein the combinatorial library encodes for a CDR1, a CDR2, and a CDR3 on a heavy chain and a CDR1, a CDR2, and a CDR3 on a light chain of the antibody.

32. A method of synthesizing a variant nucleic acid library, comprising:
a. providing predetermined sequences encoding for a plurality of polynucleotides, wherein the polynucleotides encode for a plurality of codons having a variant sequence compared to a single reference sequence;
b. selecting a distribution value for codons at a preselected position in the reference sequence;
c. providing machine instructions to randomly generate a set of nucleic acid sequences with a distribution value that aligns with the selected distribution value, wherein the set of nucleic acid sequences is less than the amount of nucleic acid sequences required to generate a saturating codon variant library; and
d. synthesizing the variant nucleic acid library with a preselected distribution, wherein at least about 70% of a predicted diversity is represented.

33. (canceled)

34. (canceled)

35. (canceled)

36. The method of claim 32, wherein the variant nucleic acid library when translated encodes for a protein library.

37. (canceled)

38. The method of claim 32, further comprising performing PCR mutagenesis of a nucleic acid using the variant nucleic acid library as primers for a PCR mutagenesis reaction.

39. The method of claim 32, wherein a codon assignment is used for determining each codon of the plurality of codons having a variant sequence.

40. The method of claim 39, wherein the codon assignment is based on frequency of the codon sequence in an organism.

41. The method of claim 40, wherein the organism is an animal, a plant, a fungus, a protist, an archaeon, a bacterium, or a combination of any of the foregoing.

42. The method of claim 39, wherein the codon assignment is based on a diversity of the codon sequence.

43.-73. (canceled)
